(19) United States
(12) Patent Application Publication
(10) Pub. No.: US 2005/0049194 A1
(43) Pub. Date: Mar. 3, 2005

(54) Use of ephrins and related molecules to regulate cellular proliferation

(76) Inventors: Jonas Frisen, Stockholm (SE); Johan Holmberg, Stockholm (SE)

Correspondence Address: Mintz, Levin, Cohn, Ferris,;Glovsky and Popeo, P.C., The Chrysler Center, 666 Third Avenue, 24th Floor, New York, NY, 10017, US

(21) Appl. No.: 10/698,907

(22) Filed: Oct. 31, 2003

Related U.S. Application Data
(63) Continuation-in-part of application No. 10/291,290, filed on Nov. 8, 2002.
(60) Provisional application No. 60/460,488, filed on Apr. 3, 2003.
(60) Provisional application No. 60/393,272, filed on Jul. 2, 2002.
(60) Provisional application No. 60/345,206, filed on Nov. 9, 2001.

(51) Int. Cl. A61K 38/17

(52) U.S. Cl. 514/12

(57) ABSTRACT

Disclosed are nucleic acids, peptides, proteins, fusion proteins, antibodies, affibodies, and other reagents that disrupt interactions between ephrins and ephrin receptors. Specifically disclosed are reagents comprising soluble ephrins and soluble ephrin receptors. Also disclosed are methods of using these reagents for the increasing or decreasing cellular proliferation, for example, for alleviation, prevention, or treatment of one or more symptoms of a disease or disorder, including a disease or disorder of the gastrointestinal tract, reproductive tract, skin, or hematopoietic system.

### RELATED APPLICATIONS

This application is a continuation-in part of U.S. Ser. No. 60/460,488, filed Apr. 3, 2003 and of U.S. Ser. No. 10/291,290 filed Nov. 8, 2002, which is a continuation-in-part of U.S. Ser. No. 60/393,272, filed Jul. 2, 2002 and U.S. Ser. No. 60/345,206 filed Nov. 9, 2001. The present disclosure claims the benefit of priority to these applications. These applications are incorporated by reference herein in their entirety.

### FIELD OF THE INVENTION

This application is directed to nucleic acids, peptides, proteins, fusion proteins, antibodies, affibodies, and other reagents that disrupt interactions between ephrins and ephrin receptors. Further, this application is directed to methods of using these reagents for the alleviation, prevention, or treatment of one or more symptoms of a disease or disorder, including a disease or disorder of the gastrointestinal tract, reproductive tract, skin, and blood.

### BACKGROUND OF THE INVENTION

Cells are continuously replaced from stem cells in many tissues in the adult organism. The rate of cell replacement needs to be tightly controlled; decreased production can cause atrophy and an increased production can cause tumorigenesis. The regulation of cell regeneration from stem cells may be controlled at several levels, including for example, the proliferation, survival, and/or elimination rates of stem cells and their progeny. A growing line of evidence has shown that the adult mammalian brain contains stem cells capable of renewing neuronal populations. (Altman and Das, 1965; Kaplan, 1981; Goldman and Nottebohm, 1983; Cameron et al., 1995; Lois and Alvarez-Buylla, 1993; Luskin, 1993; Weiss et al., 1996; Eriksson et al., 1998; Gould et al., 1999; Rakic, 2002; Momma et al., 2000; Temple and Alvarez-Buylla, 1999). Adult skin, gut and blood all harbour distinct stem cell populations (Janes et al., 2002; Kondo et al., 2003; Marshman et al., 2002; Watt, 2001). In addition, there is evidence for the presence of stem cells in multiple other adult tissues, although their function and regulation is less well understood.

The best-characterized adult stem cell system is the hematopoietic system, where cell lineage and several regulatory mechanisms are well known (Weissman, 2000). Hematopoietic stem cells (HSCs) reside in the bone marrow of adult animals. A hallmark of these cells is their capacity to generate the cells of all three major lineages in blood, erythroid, lymphoid and myeloid lineages. Transplantation of whole bone marrow suspension or sorted HSCs readily reconstitutes the wrecked blood systems of lethally irradiated mice, allowing the recipient to survive (Weissman, 2000). This ability is of great clinical importance in treating various blood disorders in man. Hematopoietic stem cells are used daily in bone marrow transplants (Kondo et al., 2003).

Long-term repopulating hematopoietic stem cells (LT-HSC) are defined by their capacity to permanently reconstitute hematopoiesis in a bone marrow depleted host. They can be identified through the cell surface markers Sca-1 and c-Kit as well as the lack of other markers expressed by mature cells (denominated lineage or Lin). Such cells can be identified by FACS analysis as Sca-1⁺/c-Kit⁺/Lin⁻ (Kondo et al., 2003). Hematopoietic stem cell enriched population can also be detected within the side population using the supravital stain Hoechst-33342 (Goodell et al., 1996). The path from multipotent and self-renewing stem cell to fully differentiated progeny goes through several bifurcations where cells are committed to a more restricted fate. This hierarchy of commitment is mirrored by activities of overlapping transcription factors whose different combinations of expression specify the distinct lineages. Hematopoietic growth factors can affect differentiation and proliferation at various stages providing non-autonomous input.

Because HSCs exhibit an asymmetric mode of cell division, the total number of HSCs remains constant in the absence of injury (Cheshier et al., 1999). In the population as a whole, roughly half of the cell divisions must therefore be self-renewing. The signals that distinguish between self-renewal and differentiation are not wholly defined, but candidate molecules have been identified. Several recent studies emphasize the role for well known cell fate influencing molecules including Wnts (Austin et al., 1997; Reya et al., 2003), Notch (Karanu et al., 2000; Varnum-Finney et al., 2000) and Sonic Hedgehog (Bhardwaj et al., 2001) in increasing the HSC pool ex vivo. Retroviral mediated introduction of HOXB4 and HOXA9 prior to transplantation also enhances HSC expansion in vivo (Sauvageau et al., 1995; Thorsteinsdottir et al., 2002; Thorsteinsdottir et al., 1999). Downstream of the HSC pool, the distinct lineages exhibit increasing heterogeneity in cell surface marker expression, gene expression and proliferation kinetics (Kondo et al., 2003). The oligopotent progenitor populations identified are the: common lymphoid progenitor and myeloid (common myeloid progenitor, granulocyte-monocyte progenitor and megakaryocyte-erythrocyte progenitor) lineages (Akashi et al., 2000; Kondo et al., 2001; Kondo et al., 1997; Nakorn et al., 2003).

In the small intestine, stem cells reside in the lower region of the crypts of Lieberkühn below the villi (Marshman et al., 2002). Members of the Wnt family are key mitogens for intestinal stem cells (Pinto et al., 2003), and allow stabilization of β-catenin and its subsequent nuclear localization where it interacts with TCF transcription factors and drives the transcription of target genes such as c-myc (van de Wetering et al., 2002). Progeny of these stem cells exhibit stereotypical migration patterns. The presumptive Paneth cells move downward to the bottom of the crypt, whereas the absorptive, goblet, and enteroendocrine cells migrate upward toward the villus facing the lumen.

The skin is the largest organ of the human body, and requires continuous renewal of its outer layer. Skin stem cells are therefore important for survival. Skin contains an epidermal outer layer and an inner dermal layer of mesodermal origin. Separating the dermis from the epidermis is the basal membrane (Janes et al., 2002; Watt, 2001). The majority of cells in the epidermis are keratinocytes. A subpopulation of the epidermal cells, believed to be located in the bulge region, are stem cells and can give rise to the hair follicles, interfollicular epidermis and sebaceous glands. The numbers of cells derived from a single stem cell division is increased by intermediate transit amplifying cells. These cells divide rapidly and have a high probability of exiting the cell cycle to terminally differentiate. In vitro expansion and subsequent grafting of keratinocytes is an important treatment for severe burns (Compton et al., 1998).

Throughout development, cellular identity is determined by the action of overlapping transcription factors reflecting positional value. In the ventral neural tube a concentration gradient of sonic hedgehog (shh), emanating from the notochord and floor-plate, specifies the fate of the presumptive neurons according to their position along the dorsoventral axis (Briscoe and Ericson, 2001). One key set of genes conferring identity in both somite and rhombomere formation is the family of homeobox genes (Hox) (Krumlauf, 1994; Lumsden and Krumlauf, 1996). A combination of Hox gene expression patterns these structures along the antero-posterior axis into well defined segments (Cooke and Moens, 2002; Krumlauf, 1994; Lumsden and Krumlauf, 1996). The mediators of this activity remain unknown but mounting evidence points towards a role for ephrins and Eph receptors in establishing and maintaining borders between segments (Cooke and Moens, 2002; Durbin et al., 1998; Mellitzer et al., 1999; Xu et al., 1999; Xu et al., 1995).

The last decade of research has shown that ephrins provide cell-cell repulsive cues for growing axons and migrating cells during neuronal development. In part, ephrins are the answer as to how the vertebrate brain can be orderly wired with an immense number of connections. A number of studies have established the importance of ephrin-Eph receptor interactions for diverse developmental processes such as: rhombomere boundary formation (Cooke and Moens, 2002), creation of topographic maps in the vertebrate visual and other sensory systems (Drescher et al., 1997; Drescher et al., 1995; Feldheim et al., 2000; Feldheim et al., 1998; Frisén et al., 1998; Vanderhaeghen et al., 2000), migration of neural crest cells (Smith et al., 1997), motor neuron projections (Feng et al., 2000) and distinction of arteries from veins (Adams et al., 1999; Wang et al., 1999). All of these studies show the repulsive effect ephrins have on the encountering Eph receptor-expressing cell. This characteristic repulsive response is in the majority of cases accompanied by a phosphorylation event of the responding Eph receptor or ephrin-B ligand. In the case of topographic map formation, ephrins fit into the model set forth by Sperry in 1963, known as the chemoaffinity hypothesis (Sperry, 1963).

Eph family receptors are subdivided into two functional classes by their affinities for membrane-bound ligands of two different structural types. Receptors of the EphA subfamily, including EphA3 (Mek 4), EphA5 (Ehk-1), and others, bind ligands that are membrane-associated through glycerophosphatidylinositol (GPI) linkages, and may be released by phospholipases C and D (41). The GPI-linked ligands characterized to date are ephrin-A1, ephrin-A3, ephrin-A4, ephrin-A2 and ephrin-A5 (formerly called LERKs 1,3,4,6,& 7) (2,7,36,38). The EphB receptor subfamily members show overlapping high affinities for ligands that are transmembrane proteins, including ephrin-B1, ephrin-B2 and ephrin-B3 (formerly called LERKs 2,5 & 8) (9,10,30,39). The transmembrane spanning ligands show remarkable amino acid conservation on the carboxy terminus, implying conservation of structure important in their function, and clouding the distinction between receptors and ligands. In this disclosure, the term ephrin, unless otherwise specified, refers to any of the ephrins including at least the ephrins listed in this paragraph.

The receptors for ephrins are denominated Eph receptors and they constitute the largest family of receptor tyrosine kinases currently known. Analysis of sequence similarity and binding preferences has been used to divide the receptors and ligands into two families: the A-class and the B-class (Committee, 1997; Frisén et al., 1999; Kullander and Klein, 2002; Wilkinson, 2001). The A-ephrins are tethered to the outer leaflet of the cell membrane whereas the B-ephrins are transmembrane ligands. Within each class, the ligands and receptors exhibit a high degree of binding promiscuity, however only the EphA4 receptor can bind to B-class ephrins. (Frisén et al., 1999; Wilkinson, 2001). Like other receptor tyrosine kinases (RTKs), the Eph receptors dimerize or oligomerize upon ligand binding, and a subsequent cross-phosphorylation event allows signal transduction (van der Geer et al., 1994). In many cases, the final target of Eph signalling is the actin cytoskeleton, which mediates growth cone collapse and axon retraction (Meima et al., 1997).

With the sole exception of a splice form of ephrin-A4 (Aasheim et al., 2000), all ephrins are membrane bound which limits the range over which they exert their action. The ligands are unable to act when only the exodomains are present to form soluble ephrins. The ligands do function when presented in membrane-bound form, suggesting that they require direct cell-to-cell contact to activate their receptors (Davis et al., 1994). Membrane attachment is thought to serve to facilitate ligand dimerization or aggregation, because antibody-mediated clustering activated previously inactive soluble forms of these ligands (Davis et al., 1994). This association with the membrane is a prerequisite for another feature of these molecules: that of reversed signalling through the ligand. Reverse signalling multiplies the possible outcomes of ephrin-Eph interactions, in effect turning ligand into receptor and vice versa.

While the biological significance for this mode of signalling has been proved for the transmembrane ephrin-B ligands (Brückner et al., 1997; Cowan and Henkemeyer, 2001; Henkemeyer et al., 1996; Holland et al., 1996), in vivo evidence for reverse A-signalling is limited to the vomeronasal and olfactory systems (Cutforth et al., 2003; Knoll et al., 2001).

During the development of the vomeronasal organ, growing axons express ephrin-A5 on their journey towards the accessory olfactory bulb, which expresses the EphA6 receptor (Knoll et al., 2001). This is in contrast to the retinotectal system (Cheng et al., 1995; Drescher et al., 1995), although in both models graded receptor and ligand expression allow the creation of a topographic map. In the vomeronasal system, apical axons express high levels of ephrin-A5 and project to the anterior portion of the accessory olfactory bulb, where levels of EphA6 are high. In the light of previous in vitro assays, showing that stimulation with Eph receptors increased adhesion of ephrin-A5-expressing cells (Davy et al., 1999; Davy and Robbins, 2000), these findings support the idea that ephrin-A5 has an adhesive or attractive role. Indeed, analysis of ephrin-A5-mutant mice revealed that some apical axons (Knoll et al., 2001) went astray and terminated in the posterior part of the accessory olfactory bulb (Knoll et al., 2001). The in vivo significance of these findings remains to be evaluated, but the possibility of reverse signalling among A-class ephrins, adds another layer of complexity to an already intricate picture. Taken together, these findings provide evidence that ephrin-A5 has a functional receptor role in regulating adhesive or attractive properties of cells in different systems.

The Caenorhabditis elegans genome encodes one Eph-receptor, VAB-1, and four ephrins, EFN-1 to EFN-4 (Chin-Sang et al., 1999; George et al., 1998; Wang et al., 1999). Mutations in the ligand-binding domain of the gene encoding VAB-1 result in defective gastrulation, cleft closure and epidermal enclosure (a process resembling neurulation in vertebrates). Notably, the analysis revealed that the adhesive properties conferred by VAB-1 during ventral enclosure were independent of kinase activity, suggesting that this mode of kinase-independent adhesive function of Eph-receptors is evolutionarily conserved. Mutations in the kinase domain of the same receptor instead generated defects reminiscent of axon guidance defects in chick and mouse, instances in which the ability to mediate repulsion is crucial. The kinase-independent effects in the worm could reflect either EFN and VAB-1 acting as adhesion molecules, or reverse signalling through the EFN-ligands (Chin-Sang et al., 1999; George et al., 1998; Wang et al., 1999).

Expression patterns suggest involvement of ephrins in tissue other than the developing nervous system and vasculature. Ephrins and Eph receptors have more recently been found to be expressed in several stem cell populations in large scale stem cell microarray analyses (Ivanova et al., 2002; Ramalho-Santos et al., 2002). In the adult organism, expression is widespread in a multitude of tissues, and expression is often prominent in the stem cell compartment. The roles played by these molecules in migration and axon pathfinding (Cowan and Henkemeyer, 2002; Holmberg and Frisén, 2002; Palmer and Klein, 2003) suggest similar functions in hematopoiesis. On the other hand studies of the adult brain show direct control of stem cell/progenitor proliferation through Eph-ephrin signalling (Conover et al., 2000). A recent study sheds light on the role for EphB2, EphB3 and ephrin-B1 in the correct cell positioning in the intestinal epithelium (Batlle et al., 2002). EphB2/B3 null mutant mice suffer from an intermingling of the differentiated and proliferative populations. The deletion of the EphB2/B3 genes eliminates necessary repulsive signals, which allows non-proliferative ephrin-B1 expressing cells to migrate downward and occupy the stem cell compartment; it also displaces the postmitotic paneth cells upwards (Batlle et al., 2002).

### BRIEF SUMMARY OF THE INVENTION

One embodiment of the invention is directed to a method of modulating (e.g., blocking, interfering, or preventing) the interaction of an ephrin receptor with an ephrin ligand, and thereby altering the growth and/or proliferation of cells (e.g., stem cells or progenitor cells) in vitro or in vivo.

In one method of the invention, one or more reagents (e.g., nucleic acids, peptides, proteins, fusions proteins, antibodies, affibodies, and the like) are administered to induce or repress cell (e.g., stem cell or progenitor cell) growth, proliferation, differentiation, migration, and/or survival. The antibody may be a polyclonal or monoclonal antibody, or fragment thereof, that binds to an ephrin (e.g., ephrin-A1, A2, A3, A4, A5, B1, B2, or B3). The peptide may comprise a soluble ephrin that includes an exodomain of ephrin-A1, A2, A3, A4, A5, B1, B2, or B3, or a soluble Eph receptor. The fusion protein may comprise a soluble ephrin and a constant domain of an immunoglobulin (e.g., ephrin-A2-Fc or ephrin-B2-Fc). The cell (e.g., stem cell or progenitor cell) may be derived from or present in a tissue, such as bone marrow, or tissue of the skin, esophagus, stomach, small intestine, large intestine, rectum, prostate, testis, penis, ovaries, uterus, cervix, fallopian tubes, vulva, or vagina.

Another embodiment of the invention is directed to a method of preventing, ameliorating, alleviating, and/or treating a symptom of a disease or disorder of the gastrointestinal tract, reproductive tract, skin, hematopoietic system, or another body system.

In one method, one or more reagents of the invention (e.g., nucleic acids, peptides, proteins, fusions proteins, antibodies, affibodies, and the like) can be administered to increase the growth, proliferation, differentiation, migration, and/or survival of a cell (e.g., a stem cell or progenitor cell). The reagents can be administered in vivo to a subject suffering from the disease or disorder associated with decreased number of cells (e.g., stem cells or progenitor cells), for example, hematopoietic disorders such as hypoproliferative anemia, and other disorders described herein. In an alternate method, one or more reagents of the invention (e.g., nucleic acids, peptides, proteins, fusions proteins, antibodies, or affibodies, and the like) can be administered to decrease the growth, proliferation, differentiation, migration, and/or survival of a cell (e.g., a stem cell or a progenitor cell). The reagents can be administered in vivo to a subject suffering from a proliferative disease or disorder of the gastrointestinal tract, reproductive tract, or skin, as described herein.

Another embodiment of the invention is directed to a method of using one or more reagents (e.g., nucleic acids, peptides, proteins, fusions proteins, antibodies, or affibodies, and the like) for inducing in vitro or in vivo growth, proliferation differentiation, survival and/or migration of a cell (e.g., a stem cell or progenitor cell) derived from or located in a tissue such as bone marrow. An additional method uses one or more reagents (e.g., peptides, proteins, fusions proteins, antibodies, or affibodies, and the like) for repressing in vitro or in vivo growth, proliferation differentiation, survival and/or migration of a cell (e.g., a stem cell or progenitor cell) derived from or located in tissue of the skin, esophagus, stomach, small intestine, large intestine, rectum, prostate, testis, penis, ovaries, uterus, cervix, fallopian tubes, vulva, or vagina.

Another embodiment of the invention is directed to a method of using one or more reagents (e.g., nucleic acids peptides, proteins, fusions proteins, antibodies, or affibodies, and the like) for inducing the growth, proliferation differentiation, survival and/or migration of cells (e.g., stem cells or progenitor cells) in a hematopoietic system in a subject, comprising administering to the subject an expression vector for expressing the reagent in a therapeutically effective amount. An alternate method uses one or more reagents (e.g., nucleic acids, peptides, proteins, fusions proteins, antibodies, or affibodies, and the like) for inducing the growth, proliferation differentiation, survival and/or migration of cells (e.g., stem cells or progenitor cells) in a gastrointestinal tract, reproductive tract, or the skin in a subject, comprising administering to the subject an expression vector for expressing the reagent in a therapeutically effective amount. In these methods, the expression vector may be a non-viral expression vector. The vector may be a non-lytic viral vector, as described in detail herein.

Another embodiment of the invention is directed to a method for treating a disease or disorder of the hematopoietic system. In one method, a population of hematopoietic cells (e.g., stem cells or progenitor cells) is treated with one or more reagents of the invention (e.g., nucleic acids, peptides, proteins, fusions proteins, antibodies, or affibodies, and the like), and then administered to a subject in need of such cells. In one aspect, the method involves the steps of (a) providing a population of hematopoietic stem cells or hematopoietic progenitor cells; (b) suspending the hematopoietic stem cells or hematopoietic progenitor cells in a solution comprising a mixture comprising a reagent of the invention to generate a cell suspension; and (c) delivering the cell suspension to a hematopoietic system of the subject. An optional addition step may include the step of injecting the injection site with the growth factor for a period of time before, after, or during (co-injection) the step of delivering the cell suspension. This includes mammals (such as humans) with a disease or disorder of the hematopoietic system. In another method, a non-human mammal can be engrafted with the enriched hematopoietic stem cells or hematopoietic progenitor cells as described herein.

Another embodiment of the invention is directed to a method of blocking an ephrin receptor from interacting with an endogenous ephrin ligand on a cell (e.g., a stem cell or progenitor cell), the method comprising exposing a stem cell or progenitor cell expressing an ephrin receptor to a reagent of the invention (e.g., a peptide, protein, fusion protein, antibody, affibody, or related molecule), and thereby inducing or repressing cell (e.g., stem cell or progenitor cell) growth, proliferation differentiation, survival and/or migration. Methods that block an ephrin receptor can be used to induce cell (e.g., stem cell or progenitor cell) growth, proliferation differentiation, survival, and/or migration in hematopoietic systems. Methods that block an ephrin receptor can be used to repress cell (e.g., stem cell or progenitor cell) growth, proliferation differentiation, survival and/or migration in the gastrointestinal tract, reproductive tract, and the skin.

Another embodiment of the invention is directed to a method for treating a disorder with an abnormal level (abnormally high or abnormally low) of cellular proliferation. The proliferation may be in a stem cell or a progenitor cell. The method comprise administering to a patient suffering from this disorder an agent that interrupts the interaction of ephrin with ephrin receptors. The agent includes, at least, any reagents, soluble ephrin, soluble ephrin receptor (ligand binding domain), antibody, affibody, and small molecules, listed in this disclosure, that can disrupt an interaction between ephrin and ephrin receptor. Derivatives, oligomers, and functional equivalents of these agents are also envisioned as an agent of this method. The agent modulates (increase or decrease) cell proliferation and bring it back to a normal level. In some tissues, the disorder increases proliferation and the agent decreases proliferation. In other tissues, the disorder decreases proliferation and the agent increases proliferation. The disorder may be any disorder listed in this disclosure.

The methods of the invention can be used for human and non-human animals, and with human and non-human cells.

### BRIEF DESCRIPTION OF THE FIGURES

FIG. 1

FIG. 1A-D: Injection of 100 μg ephrin-A2-Fc or ephrin-B2-Fc increased BrdU-incorporation in both whole bone marrow and in the Sca.-1⁺/c-kit+/Thy-1 lo population. The combined injection of 100 μg ephrin-A2-Fc plus 100 μg ephrin-B2-Fc did not further increase proliferation in any of the analyzed populations. In each group n=4. FIG. 1E: Levels of injected Fc-protein in whole blood serum appeared stable over time in all groups analyzed, although ephrin-A2-Fc appeared less stable than the others. In each group n=4. FIG. 1F: Increased numbers of leukocytes were evident in the ephrin-A2-Fc injected animals as determined by leukocyte particle count (LPC). In each group n=4. FIG. 1G-H: The number of cells in two distinct lineages were increased in the ephrin-A2 null mutant: thrombocytes and leukocytes. For wild-types n=7, for ephrin-A2 null mutants n=10.

FIG. 2

FIG. 2A-C: Decreased proliferation in the crypts of Lieberkühn following injection of 100 μg ephrin-A2-Fc or ephrin-B2-Fc as determined by counting of PCNA⁺ positive cells. A similar result was obtained through counting of BrdU⁺-cells. FIG. 2A: The combined injection of 100 μg ephrin-A2-Fc plus 100 μg ephrin-B2-Fc did not further decrease the rate of proliferation. FIG. 2D: Quenching of proliferation was increased at high concentrations of ephrin-Fc. FIG. 2E-F: Position of post-mitotic Paneth cells was maintained in infused animals. FIG. 2G-H: The injected ephrin-B2-Fc chimeras selectively bound to cells in the lower proliferative compartment of the crypts. In all groups n=4.

FIG. 3

Overall structure of the small intestine was affected in the EphB2/B3 null mutant E18 embryos (FIG. 3B, E) and in the EphB3 null/EphB2Δ/Δ (FIG. 3C, D) compared to wild type embryos (FIG. 3A, D). Lower number of PCNA⁺ cells in the villi of the E18 mutant embryos was indicative of attenuated proliferation (FIG. 3G). The total number of cells in villi of the EphB3 null/EphB2Δ/Δ mutant but not in the double null mutant was reduced (FIG. 3H). In each group n=4.

FIG. 4

FIG. 4A: In the adult intestinal epithelium the crypts of Lieberkühn harbors the stem cell population responsible for the rapid turnover of differentiated cells. To facilitate the counting of cells in the crypt, it was divided into compartments: SC indicates the side compartment, PC indicates the Paneth cell compartment, and TC indicates the total cell compartment encompassing both SC and PC. FIG. 4B, E, H-1: In the wild type crypts proliferation was mainly confined to the SC whereas the PC revealed a low number of PCNA⁺-cells. Total number of cells was also slightly lower in the PC. FIG. 4C, F, H-1: In the EphB2/B3 mutant mice cells were displaced from the SC to the PC were they proliferate in response to the high levels of Wnt in the PC. FIG. 4D, G, H-1: EphB3 null/EphB2Δ/Δ mutants displayed a similar phenotype. The primary diminishing of proliferation in the mutants was lessened by the displacement of non-proliferating cells to the PC where the cells enter the cell cycle in response to Wnt. FIG. 4J: In animals exposed to only 24 h of ephrin-B2-Fc, the proliferation was significantly altered. In all groups n=4, except in (J) where n=5.

FIG. 5

None of the tissues showed amplification of effects by combined injections of ephrin-A2-Fc and ephrin-B2-Fc. The tissues analyzed included whole bone marrow (FIG. 5A), Sca-1⁺/c-kit⁺ cells from bone marrow (FIG. 5B), lateral wall of the lateral ventricle in the brain of intraventriculary infused animals (FIG. 5C) and crypts of Lieberkühn in small intestine (FIG. 5D). In all groups n=4, except A2 and Fc in (C) where n=3.

FIG. 6

FIG. 6A: The BrdU-incorporation in the stem cell population of skin was severely reduced in ephrin-A2-Fc and ephrin-B2-Fc injected animals. A dose dependent response was clearly visible in ephrin-A2-Fc infused animals, the injected amounts were 1, 10 & 100 μg of fusion protein. FIG. 6B: As control 100 μg of Fc protein was injected. As an additional control one group of mice received PBS, which produced a similar result as the Fc-control. In all groups n=4.

FIG. 7

Depiction of embryonic intervillus epitehlium and adult crypt cell populations in wild-type and Eph mutants. Circular arrows indicate proliferating cells. Further described in Example 3, below.

FIG. 8

Effects of the administration of recombinant ephrin-A2-Fc and recombinant ephrin-B2-Fc, alone or combined, on proliferation of bone marrow cells. Column 1: Results for recombinant human IgG-Fc protein. Column 2: Results for the combination of recombinant mouse ephrin-A2-Fc and recombinant mouse ephrin-B2-Fc. Column 3: Results for recombinant mouse ephrin-A2-Fc. Column 4: Results for recombinant mouse ephrin-B2-Fc.

FIG. 9

Effects of the administration of recombinant ephrin-A2-Fc and recombinant ephrin-B2-Fc, alone or combined, on proliferation of hematopoietic stem cells containing markers for newborn cells. Column 1: Results for recombinant human IgG-Fc protein. Column 2: Results for the combination of recombinant mouse ephrin-A2-Fc and recombinant mouse ephrin-B2-Fc. Column 3: Results for recombinant mouse ephrin-A2-Fc. Column 4: Results for recombinant mouse ephrin-B2-Fc.

FIG. 10

GenBank numbers, annotations, and amino acid sequences for mouse ephrin-A1 (SEQ ID NO:1), A2 (SEQ ID NO:2), A3 (SEQ ID NO:3), A4 (SEQ ID NO:4), and A5 (SEQ ID NO:5). The regions of the conserved domains (pfam00812.9; ephrin) are indicated.

FIG. 11

GenBank numbers, annotations, and amino acid sequences for mouse ephrin-B 1 (SEQ ID NO:6), B2 (SEQ ID NO:7), and B3 (SEQ ID NO:8). The regions of the conserved domains (pfam00812.9; ephrin) are indicated.

FIG. 12

GenBank numbers, annotations, and amino acid sequences for human ephrin-A1 (SEQ ID NO:9), A2 (SEQ ID NO:10), A3 (SEQ ID NO:11), A4 (SEQ ID NO:12), and A5 (SEQ ID NO:13). The regions of the conserved domains (pfam00812.9; ephrin) are indicated.

FIG. 13

GenBank numbers, annotations, and amino acid sequences for human ephrin-B 1 (SEQ ID NO:14), B2 (SEQ ID NO:15), and B3 (SEQ ID NO:16). The regions of the conserved domains (pfam00812.9; ephrin) are indicated.

FIG. 14

Amino acid sequences for GST-EphA7-LBD (SEQ ID NO: 17), mouse ephrin-A2 exodomain (SEQ ID NO: 18), human ephrin-A2 exodomain (SEQ ID NO: 19), mouse ephrin-B2 exodomain (SEQ ID NO:20), and human ephrin-B2 exodomain (SEQ ID NO:21). GenBank number, annotations, and amino acid sequence for human IgG1 (SEQ ID NO:22).

### DETAILED DESCRIPTION OF THE INVENTION

The Eph tyrosine kinase receptors and their ephrin ligands confer short range communication between cells in the developing organism regulating diverse processes such as axon guidance, cell migration and neural tube formation (Wilkinson, D. G., 2001. Nat Rev Neurosci 2(3): 155-64). Even though both receptors and ligands are widely expressed in the adult nervous system, the knowledge concerning their roles in adult tissues is limited. Neurogenic areas in the adult brain, including the lateral wall of the lateral ventricle and the dentate gyrus of the hippocampus, express EphA7 and the ligands ephrin-A2. Mice lacking the receptor EphA7 exhibit increased cellular proliferation in the tissue on the lateral side of the lateral ventricle.

It has been previously shown in the wild type organism the ephrin or Eph are negative regulators of proliferation, keeping it at a basal level (see U.S. Ser. No. 10/291,290 filed Nov. 8, 2002). This effect involves reversed signaling through the ligand upon binding to the EphA7 receptor. Upon injection of the freely soluble form of ephrin-A5-Fc, ephrin-A2, or EphA7 either as monomers or as oligomers into the lateral ventricle, the number of proliferating cells as measured by BrdU-labeling was significantly higher than in sham-injected mice (see U.S. Ser. No. 10/291,290 filed Nov. 8, 2002). The ephrin-A5-Fc, ephrin-A2 or EphA7 proteins disrupted the binding between the endogenous ligands and receptors, thereby blocking signaling through the ligands, and allowing a higher rate of proliferation.

In the experiments described herein, the roles of ephrins and Eph receptors were analyzed in several stem cell populations in the adult. Eph-ephrin signaling was blocked by the administration of soluble ephrin-Fc fusion proteins, or in mice carrying mutations in ephrin or Eph receptor genes. Similar to the observations made for the brain, ephrins were found to negatively regulate proliferation of hematopoietic stem cells. In contrast, ephrins were found to act as positive regulators in stem cell populations in the intestine and skin. In all these tissues, blocking A- or B-class ephrins and Eph receptors were found to have parallel effects. Blocking both classes simultaneously, however, did not result in an additive or synergistic effect. From these experiments, it was concluded that ephrins and Eph receptors differentially regulate proliferation in different stem cell populations in the adult, and that Eph-ephrin pathway can be used to stimulate or inhibit cell regeneration.

Production Of Reagents

Included in the invention are reagents comprising a soluble ephrin, which includes an exodomain (i.e., extracellular domain) or a fragment thereof from one or more ephrins, such as ephrin-A1, A2, A3, A4, A5, B1, B2, and B3. Also included are reagents comprising a soluble ephrin receptor, which includes a ligand binding domain, or fragment thereof, from one or more ephrin receptors, such as EphA1, EphA2, EphA3, EphA4, EphA5, EphA7, EphA8, EphB1, EphB2, EphB3, EphB4, and EphB6. Preferred are soluble ephrin-A2 and B2. Preferred are soluble EphA7. Further included as reagents are fusion proteins comprising a soluble ephrin (e.g., ephrin-A2-Fc and ephrin-B2-Fc), anti-ephrin antibodies or affibodies, anti-Eph antibodies or affibodies, and any related molecules that can interfere with Eph interactions. As used herein, the term “reagent” refers to any substance that is chemically and biologically capable of blocking, preventing, or attenuating interaction of an ephrin with an ephrin receptor, including nucleic acids, peptides, proteins, fusion proteins, small molecules, antibodies (or fragments thereof), affibodies, and the like.

In various aspects of the invention, a soluble ephrin can include an amino acid sequence (including an exodomain) of one or more of the following (GenBank numbers and names are indicated): EFA1_HUMAN P20827 Ephrin-A1 precursor (EPH-related receptor tyrosine kinase ligand 1) (LERK-1) (Immediate early response protein B61) [205 residues]; EFA1_MOUSE P52793 Ephrin-A1 precursor (EPH-related receptor tyrosine kinase ligand 1) (LERK-1) (Immediate early response protein B61). [205 residues]; EFA1_RAT P97553 Ephrin-A1 precursor (EPH-related receptor tyrosine kinase ligand 1) (LERK-1) (Immediate early response protein B61) [205 residues]; EFA1_XENLA P52794 Ephrin-A1 precursor (EPH-related receptor tyrosine kinase ligand 1) (LERK-1) (XELF-a) [216 residues]; EFA2_BRARE P79727 Ephrin-A2 precursor (EPH-related receptor tyrosine kinase ligand 6) (LERK-6) (ELF-1) (ZFEPHL3) 195 residues]; EFA2_CHICK P52802 Ephrin-A2 precursor (EPH-related receptor tyrosine kinase ligand 6) (LERK-6) (ELF-1) [200 residues]; EFA2_HUMAN 043921 Ephrin-A2 precursor (EPH-related receptor tyrosine kinase ligand 6) (LERK-6) (HEK7-ligand) (HEK7-L) [213 residues]; EFA2_MOUSE P52801 Ephrin-A2 precursor (EPH-related receptor tyrosine kinase ligand 6) (LERK-6) (ELF-1) (CEK7-ligand) (CEK7-L) [209 residues].

Also included are EFA3_HUMAN P52797 Ephrin-A3 precursor (EPH-related receptor tyrosine kinase ligand 3) (LERK-3) (EHK1 ligand) (EHK1-L) [238 residues]; EFA3_MOUSE 008545 Ephrin-A3 (EPH-related receptor tyrosine kinase ligand 3) (LERK-3) (EHK1 ligand) (EHK1-L) [187 residues]; EFA4_HUMAN P52798 Ephrin-A4 precursor (EPH-related receptor tyrosine kinase ligand 4) (LERK-4) [201 residues]; EFA4_MOUSE 008542 Ephrin-A4 precursor (EPH-related receptor tyrosine kinase ligand 4) (LERK-4) [206 residues]; EFA5_BRARE P79728 Ephrin-A5 precursor (EPH-related receptor tyrosine kinase ligand 7) (LERK-7) (AL-1) (ZFEPHL4) [228 residues]; EFA5_CHICK P52804 Ephrin-A5 precursor (EPH-related receptor tyrosine kinase ligand 7) (LERK-7) (RAGS protein) [228 residues]; EFA5_HUMAN P52803 Ephrin-A5 precursor (EPH-related receptor tyrosine kinase ligand 7) (LERK-7) (AL-1). Ephrin-A5 precursor (EPH-relate [228 residues]; EFA5_MOUSE 008543 Ephrin-A5 precursor (EPH-related receptor tyrosine kinase ligand 7) (LERK-7) (AL-1). Ephrin-A5 precursor (EPH-relate [228 residues]; EFA5_RAT P97605 Ephrin-A5 precursor (EPH-related receptor tyrosine kinase ligand 7) (LERK-7) (AL-1) [228 residues]; EFB1_CHICK 073612 Ephrin-B1 precursor (CEK5 ligand) (CEL5-L). Ephrin-B1 precursor (CEK5 ligand) (CEL5-L) [334 residues].

Further included are EFB1_HUMAN P98172 Ephrin-B 1 precursor (EPH-related receptor tyrosine kinase ligand 2) (LERK-2) (ELK ligand) (ELK-L) [346 residues]; EFB1_MOUSE P52795 Ephrin-B1 precursor (EPH-related receptor tyrosine kinase ligand 2) (LERK-2) (ELK ligand) (ELK-L) (STRA1 protein) [345 residues]; EFB1_RAT P52796 Ephrin-B1 precursor (EPH-related receptor tyrosine kinase ligand 2) (LERK-2) (ELK ligand) (ELK-L) [345 residues]; EFB1_XENLA 013097 Ephrin-B1 precursor (EPH-related receptor tyrosine kinase ligand 2) (LERK-2) (ELK ligand) (ELK-L) (XLERK). [327 residues]; EFB2_BRARE 073874 Ephrin-B2 precursor (Ephrin B2a). Ephrin-B2 precursor (Ephrin B2a). [332 residues]; EFB2_HUMAN P52799 Ephrin-B2 precursor (EPH-related receptor tyrosine kinase ligand 5) (LERK-5) (HTK ligand) (HTK-L) [333 residues]; EFB2_MOUSE P52800 Ephrin-B2 precursor (EPH-related receptor tyrosine kinase ligand 5) (LERK-5) (HTK ligand) (HTK-L) (ELF-2) [336 residues]; EFB3_HUMAN Q15768 Ephrin-B3 precursor (EPH-related receptor tyrosine kinase ligand 8) (LERK-8) [340 residues]; EFB3_MOUSE 035393 Ephrin-B3 precursor [340 residues]; 042304 Ephrin-A5 (Fragment) [80 residues]; 044516 Hypothetical 39.6 kDa protein (Ephrin EFN-4). [348 residues].

Additionally included are 093431 Ephrin A-L1 [229 residues]; Q19475 F15A2.5 protein [155 residues]; Q8N578 Ephrin-A1 [205 residues]; Q90YC5 Ephrin-A3 [219 residues]; Q90Z31 Ephrin B3. [331 residues]; Q90Z32 Ephrin B2b. [334 residues]; Q90Z33 Ephrin B1 [341 residues]; Q923G4 Ephrin A3 (Fragment). Ephrin A3 (Fragment). [118 residues]; Q98TZ1 Ephrin-A6 (Fragment) [202 residues]; Q9CZS8 10 days embryo cDNA, RIKEN full-length enriched library, clone:2610529M21, full insert sequence [206 residues]; Q9D7K8 Adult male tongue cDNA, RIKEN full-length enriched library, clone:2310004J15, full insert sequence [205 residues]; Q9PT69 Ephrin-B3 precursor. Ephrin-B3 precursor. [327 residues]; Q9PTD0 Ephrin A3 (Fragment) [88 residues]; Q9PTD1 Ephrin A2 (Fragment) [93 residues]; Q9PUJ4 Ephrin-B2 precursor [333 residues]; Q9U3M2 C43F9.8 protein [237 residues]; Q9U474 VAB-2 (Hypothetical protein Y37E11AR.6) [279 residues]; Q9V4E1 Ephrin protein [652 residues]; Q9W6H9 Ephrin-B2 (Fragment) [205 residues]; Q9WUE7 Ephrin A-2 (Fragment) [102 residues].

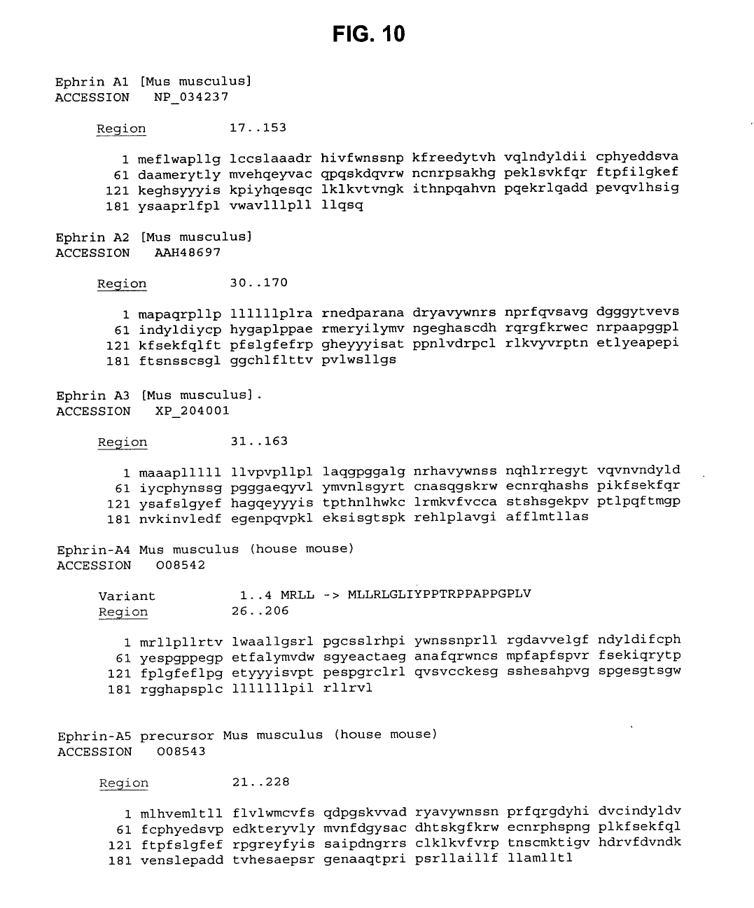

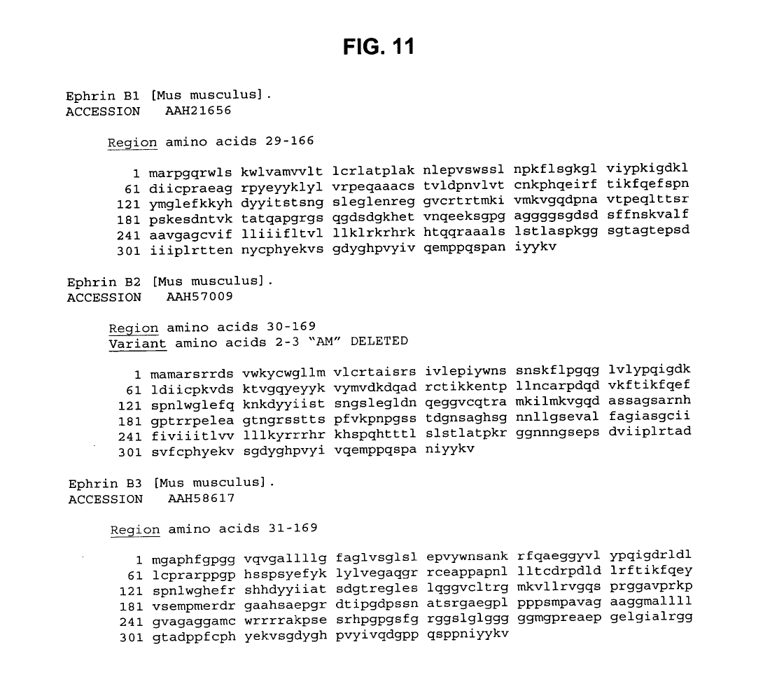

In accordance with methods of the invention, the mouse ephrin-A1 exodomain can be used, including Met 1 to Ser 182 (amino acids 1-182), or fragments thereof; the mouse ephrin-A2 exodomain can be used, including Met 1 to Asn 184 (amino acids 1-184), or fragments thereof; the mouse ephrin-A3 exodomain can be used, including Met 1 to Ser 203 (amino acids 1-203), or fragments thereof; the mouse ephrin-A4 exodomain can be used including Met 1 to Gly 176 (amino acids 1-176), or fragments thereof; the mouse ephrin-A5 exodomain can be used, including Met 1 to Asn 203 (amino acids 1-203), or fragments thereof; the mouse ephrin-B1 exodomain can be used, including Met 1 to Ser 229 (amino acids 1-229) or fragments thereof; the mouse ephrin-B2 exodomain can be used, including Met 1 to Ala 229 (amino acids 1-229), or fragments thereof, or if the variant sequence is used (see FIG. 11) Met 1 to Ala 227 (amino acids 1-227); the mouse ephrin-B3 exodomain can be used, including Met 1 to Ser 224 (amino acids 1-224), or fragments thereof (see FIG. 10-11).

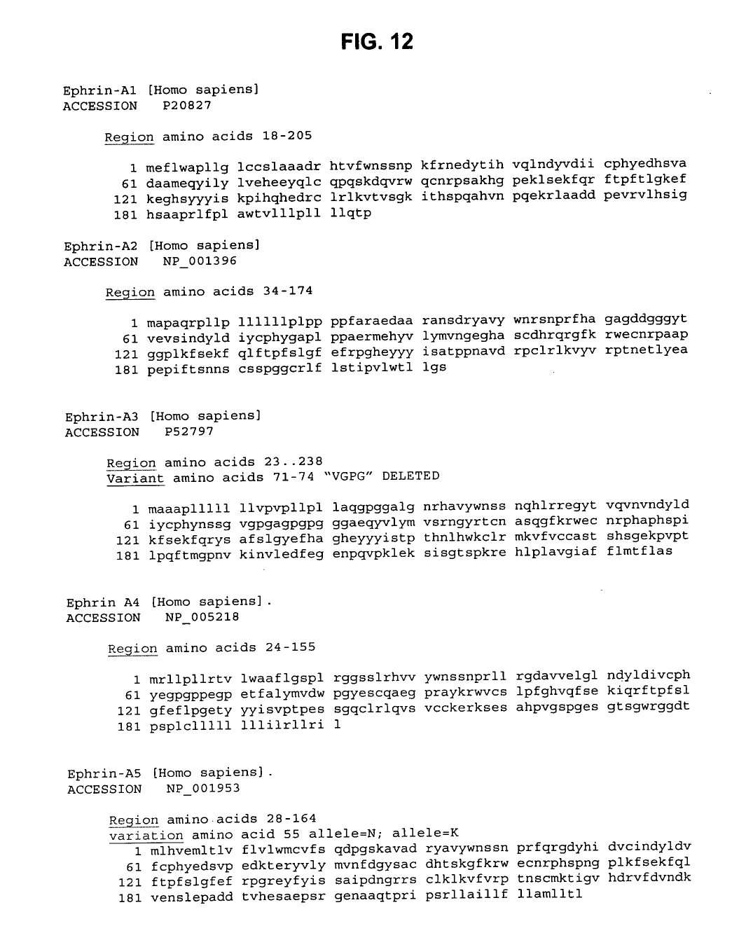

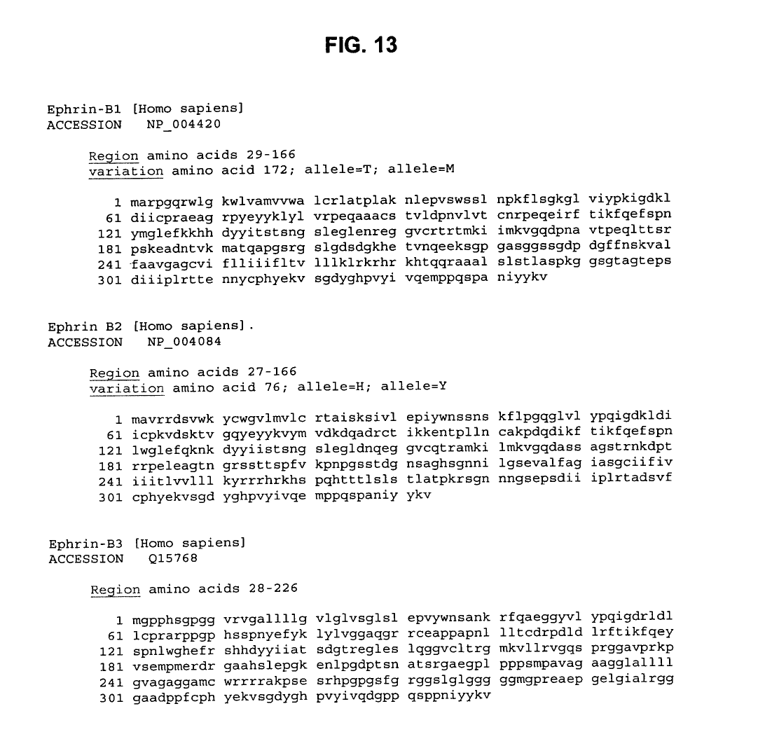

In addition, the human ephrin-A1 exodomain can be used, incluing Met 1 to Ser 182 (amino acids 1-182), or fragments thereof; the human ephrin-A2 exodomain can be used, including Met 1 to Asn 188 (amino acids 1-188), or fragments thereof; the human ephrin-A3 exodomain can be used, including Met 1 to Ser 211 (amino acids 1-211), or fragments thereof, or if the variant sequence is used (see FIG. 12), Met 1 to Ser 209 (amino acids 1-209), or fragments thereof; the human ephrin-A4 exodomain can be used, including Met 1 to Gly 171 (amino acids 1-171), or fragments thereof; the human ephrin-A5 exodomain can be used, including Met 1 to Asn 203 (amino acids 1-203), or fragments thereof; the human ephrin-B1 exodomain, incluing Met 1 to Pro 230 (amino acids 1-230) or fragments thereof; the human ephrin-B2 exodomain, including Met 1 to Ala 226 (amino acids 1-226), or fragments thereof;
the human ephrin-B3 exodomain can be used, including Met 1 to Ser 224 (amino acids 1-224), or fragments thereof (see FIG. 12-13). The exodomains of ephrins are generally known in the art and have been previously published (see, e.g., Takahashi et al., 1995, Oncogene 11:879; Kozlosky et al., 1995, Oncogene 10:229; Davis et al., 1994, Science 266:816; Cerretti et al., 1998, Genomics 47:131; Shao et al., 1995, J. Biol. Chem. 270:3467; Hirai, H. et al., 1987, Science 238: 1717-1720; Specifications and Use documents, R&D Systems, Inc.).

For use with the methods of invention are soluble ephrin receptors comprising ligand binding domains, or fragments thereof, as can be easily determined by one of skill in the art based on the disclosure herein and available publications. For example, the sequence of the ephrin receptors may be determined from GenBank. The LBD of the ephrin receptors are listed in the GenBank entries. These soluble ephrin receptors may be used as a substitute for any soluble ephrin in this disclosure for any of the methods of this disclosure. For example, like the soluble ephrin, the soluble ephrin receptors may be in the form of a hybrid polypeptide comprising, for example, the ligand binding domain linked to the constant region of an immunoglobulin molecule. These ligand binding domains (LBDs) has the desired biological effect (as shown in the Examples) and would include, at least, the following:

| | GenBank | LBD (amino |
|---|---|---|
| Ephrin Receptor [Species] | Accession No. | acid positions) |
| EphA1 [Homo sapiens] | NP_005223 | 27-204 |
| EphA2 [Homo sapiens] | NP_004422 | 28-201 |
| EphA3 [Homo sapiens] | NP_005224 | 29-202 |
| EphA4 [Homo sapiens]. | NP_004429 | 30-204 |
| EphA5 [Homo sapiens]. | NP_872272 | 60-233 |
| EphA7; [Homo sapiens] | NP_004431 | 32-205 |
| EphA8 [Homo sapiens] | NP_065387 | 31-204 |
| EphB1 [Homo sapiens] | NP_004432 | 19-196 |
| EphB2 [Homo sapiens] | NP_004433 | 20-197 |
| EphB3 [Homo sapiens] | NP_004434 | 39-212 |
| EphB4 [Homo sapiens] | NP_004435 | 29-197 |
| EphB6 [Homo sapiens] | NP_004436 | 23-217 |

Reagents such as peptides and proteins can be produced, purified and formulated according to well known methods. In one aspect, reagents of the invention, and individual moieties or analogs and derivatives thereof, can be chemically synthesized. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989).

The peptides and proteins of the invention can be purified so that they are substantially free of chemical precursors or other chemicals using standard purification techniques. The language “substantially free of chemical precursors or other chemicals” includes preparations in which the peptide or protein is separated from chemical precursors or other chemicals that are involved in synthesis. In one embodiment, the language “substantially free of chemical precursors or other chemicals” includes preparations having less than about 30% (by dry weight) of chemical precursors or other chemicals, more preferably less than about 20% chemical precursors or other chemicals, still more preferably less than about 10% chemical precursors or other chemicals, and most preferably less than about 5% chemical precursors or other chemicals.

Chemical synthesis of peptides and proteins can be used for the incorporation of modified or unnatural amino acids, including D-amino acids and other small organic molecules. Replacement of one or more L-amino acids in a peptide or protein with the corresponding D-amino acid isoforms can be used to increase resistance to enzymatic hydrolysis, and to enhance one or more properties of biological activity, i.e., receptor binding, functional potency or duration of action. See, e.g., Doherty, et al., 1993. J. Med. Chem. 36: 2585-2594; Kirby, et al., 1993, J. Med. Chem. 36:3802-3808; Morita, et al., 1994, FEBS Lett. 353: 84-88; Wang, et al., 1993 Int. J. Pept. Protein Res. 42: 392-399; Fauchere and Thiunieau, 1992. Adv. Drug Res. 23: 127-159.

Introduction of covalent cross-links into a peptide or protein sequence can conformationally and topographically constrain the peptide backbone. This strategy can be used to develop peptide or protein analogs of reagents with increased potency, selectivity, and stability. A number of other methods have been used successfully to introduce conformational constraints into amino acid sequences in order to improve their potency, receptor selectivity, and biological half-life. These include the use of (i) C_{α}-methylamino acids (see, e.g., Rose, et al., Adv. Protein Chem. 37: 1-109 (1985); Prasad and Balaram, CRC Crit. Rev. Biochem., 16: 307-348 (1984)); (ii) N_{α}-methylamino acids (see, e.g., Aubry, et al., Int. J. Pept. Protein Res., 18: 195-202 (1981); Manavalan and Momany, Biopolymers, 19: 1943-1973 (1980)); and (iii) α,β-unsaturated amino acids (see, e.g., Bach and Gierasch, Biopolymers, 25: 5175-S192 (1986); Singh, et al., Biopolymers, 26: 819-829 (1987)). These and many other amino acid analogs are commercially available, or can be easily prepared. Additionally, replacement of the C-terminal acid with an amide can be used to enhance the solubility and clearance of a peptide or protein.

Alternatively, a reagent may be obtained by methods well-known in the art for recombinant peptide or protein expression and purification. A DNA molecule encoding the reagent can be generated. The DNA sequence is known or can be deduced from the amino acid sequence based on known codon usage. See, e.g., Old and Primrose, Principles of Gene Manipulation 3^{rd} ed., Blackwell Scientific Publications, 1985; Wada et al., Nucleic Acids Res. 20: 2111-2118(1992). Preferably, the DNA molecule includes additional sequences, e.g., recognition sites for restriction enzymes which facilitate its cloning into a suitable cloning vector, such as a plasmid. Nucleic acids may be DNA, RNA, or a combination thereof. Nucleic acids encoding the reagent may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like). Nucleic acids can also be generated by chemical synthesis.

Any of the methodologies known within the relevant art regarding the insertion of nucleic acid fragments into a vector may be used to construct expression vectors that contain a chimeric gene comprised of the appropriate transcriptional/translational control signals and reagent-coding sequences. Promoter/enhancer sequences within expression vectors may use plant, animal, insect, or fungus regulatory sequences, as provided in the invention.

A host cell can be any prokaryotic or eukaryotic cell. For example, the peptide can be expressed in bacterial cells such as E. coli, yeast, insect cells, fungi or mammalian cells (such as Chinese hamster ovary cells (CHO) or COS cells). Other suitable host cells are known to those skilled in the art. In one embodiment, a nucleic acid encoding a reagent is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195).

The host cells can be used to produce (i.e., overexpress) peptide in culture. Accordingly, the invention further provides methods for producing the peptide using the host cells of the invention. In one embodiment, the method comprises culturing the host cell of invention (into which a recombinant expression vector encoding the peptide or protein has been introduced) in a suitable medium such that peptide is produced. The method further involves isolating peptide or protein from the medium or the host cell. Ausubel et al., (Eds). In: Current Protocols in Molecular Biology. J. Wiley and Sons, New York, N.Y. 1998.

An “isolated” or “purified” recombinant peptide or protein, or biologically active portion thereof, is substantially free of cellular material or other contaminating proteins from the cell or tissue source from which it is derived. The language “substantially free of cellular material” includes preparations in which the peptide or protein is separated from cellular components of the cells from which it is isolated or recombinantly produced. In one embodiment, the language “substantially free of cellular material” includes preparations of peptide or protein having less than about 30% (by dry weight) of product other than the desired peptide or protein (also referred to herein as a “contaminating protein”), more preferably less than about 20% of contaminating protein, still more preferably less than about 10% of contaminating protein, and most preferably less than about 5% contaminating protein. When the peptide or protein, or biologically active portion thereof, is recombinantly produced, it is also preferably substantially free of culture medium, i.e., culture medium represents less than about 20%, more preferably less than about 10%, and most preferably less than about 5% of the volume of the peptide or protein preparation.

The invention also pertains to variants of a reagent of the invention that function as either agonists (mimetics) or as antagonists. Variants of a reagent can be generated by mutagenesis, e.g., discrete point mutations. An agonist of a reagent can retain substantially the same, or a subset of, the biological activities of the naturally occurring form of the reagent. An antagonist of the reagent can inhibit one or more of the activities of the naturally occurring form of the reagent by, for example, competitively binding to the receptor. Thus, specific biological effects can be elicited by treatment with a variant with a limited function. In one embodiment, treatment of a subject with a variant having a subset of the biological activities of the naturally occurring form of the reagent has fewer side effects in a subject relative to treatment with the naturally occurring form of the reagent.

Preferably, the analog, variant, or derivative reagent is functionally active. As utilized herein, the term “functionally active” refers to species displaying one or more known functional attributes of an unmodified reagent. “Variant” refers to a reagent differing from naturally occurring reagent, but retaining essential properties thereof. Generally, variants are overall closely similar, and in many regions, identical to the naturally occurring reagent.

Variants of the reagent that function as either agonists (mimetics) or as antagonists can be identified by screening combinatorial libraries of mutants of the reagent for peptide or protein agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual peptides, or alternatively, as a set of larger fusion proteins (e.g., for phage display) containing the set of sequences therein. There are a variety of methods that can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl. Acids Res. 11:477.

Derivatives and analogs of a reagent of the invention or individual moieties can be produced by various methods known within the art. For example, the amino acid sequences may be modified by any number of methods known within the art. See e.g., Sambrook, et al., 1990. Molecular Cloning: A Laboratory Manual, 2nd ed., (Cold Spring Harbor Laboratory Press; Cold Spring Harbor, N.Y.). Modifications include: glycosylation, acetylation, phosphorylation, amidation, derivatization by known protecting/blocking groups, linkage to an antibody molecule or other cellular reagent, and the like. Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Derivatives and analogs may be full length or other than full length, if said derivative or analog contains a modified nucleic acid or amino acid, as described infra. Derivatives or analogs of the reagent include, but are not limited to, molecules comprising regions that are substantially homologous in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art (e.g., Wisconsin GCG software) or (iii) the encoding nucleic acid is capable of hybridizing to a sequence encoding the aforementioned peptides under stringent (preferred), moderately stringent, or non-stringent conditions. See, e.g., Ausubel, et al., Current Protocols in Molecular Biology, John Wiley and Sons, New York, N.Y., 1993.

Derivatives of a reagent of the invention may be produced by alteration of their sequences by substitutions, additions, or deletions that result in functionally-equivalent molecules. One or more amino acid residues within the reagent may be substituted by another amino acid of a similar polarity and net charge, thus resulting in a silent alteration. Conservative substitutes for an amino acid within the sequence may be selected from other members of the class to which the amino acid belongs. For example, nonpolar (hydrophobic) amino acids include alanine, leucine, isoleucine, valine, proline, phenylalanine, tryptophan, and methionine. Polar neutral amino acids include glycine, serine, threonine, cysteine, tyrosine, asparagine, and glutamine. Positively charged (basic) amino acids include arginine, lysine, and histidine. Negatively charged (acidic) amino acids include aspartic acid and glutamic acid.

The reagent can be administered locally to any loci implicated in a disorder of hematopoiesis or a proliferative disorder of the gastrointestinal tract, skin, or reproductive tract. For example, the reagent can be administered locally to the bone marrow, skin, ovaries, uterus, fallopian tubes, esophagus, stomach, small intestine, large intestine, or rectum.

Hematopoietic cells (e.g., stem cells and their progeny) can be induced to proliferate and differentiate in vivo by administering to the host a reagent of the invention, alone or in combination with other agents, or by administering a pharmaceutical composition containing the reagent that will induce proliferation and differentiation of the cells. Such in vivo manipulation and modification of these cells allows cells lost, due to injury or disease, to be endogenously replaced, thus obviating the need for transplanting foreign cells into a patient. Alternatively, proliferative disorders of the gastrointestinal tract, reproductive tract, or skin (e.g., tumors or various neoplasms) can be prevented or treated by administration of a reagent of the invention, alone or in combination with other anti-proliferative agents. Pharmaceutical compositions include any reagents of the invention that block or stimulate cells (e.g., stem cells) as described herein.

Fusion Proteins

Included in the invention are reagents comprising an ephrin polypeptide sequence, for example, an exodomain, or fragment thereof from one or more ephrin, such as ephrin-A1, A2, A3, A4, A5, B1, B2, and B3, which forms a soluble ephrin. Preferred are soluble ephrin-A2 and B2. While the discussion below is directed to ephrins, it is understood that it is equally applicable to soluble ephrin receptors. In one aspect of the invention, the reagents disclosed herein can be expressed as fusion proteins. For example, the fusion protein can comprise a soluble ephrin fused to a constant region of an immunoglobulin. A constant region (i.e., Fc region) includes the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

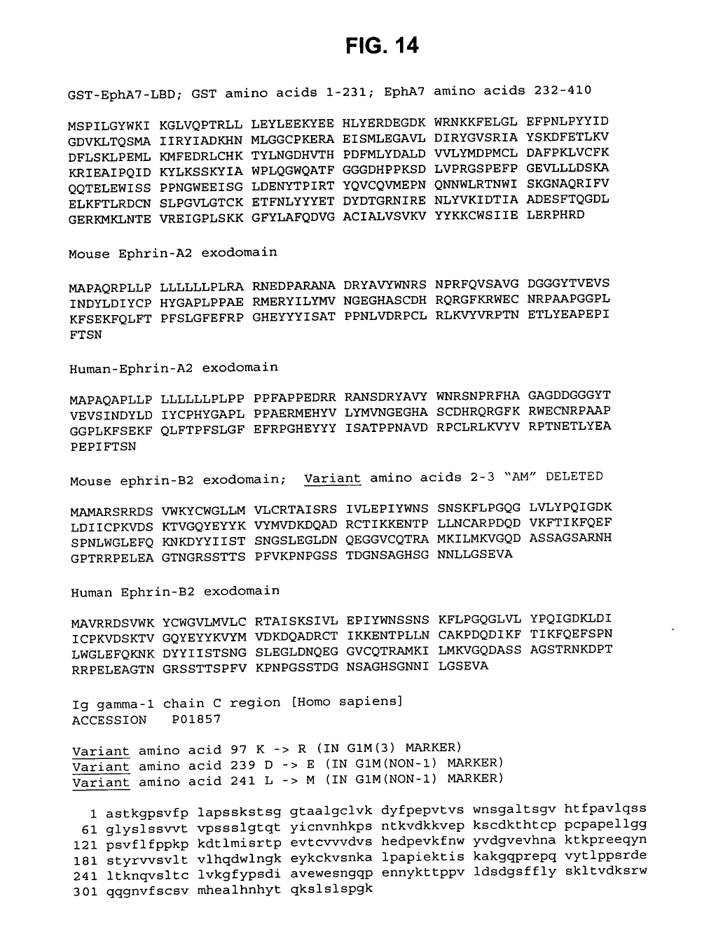

As known in the art, each immunoglobulin heavy chain constant region comprises four or five domains, including domains include CH1-hinge-CH2—CH3(—CH4) (reviewed in published U.S. Patent Application 2002/0081664). The preferred class of immunoglobulin from which the heavy chain constant region is derived is IgG (e.g., subclasses 1, 2, 3, or 4). Other classes of immunoglobulins, e.g., IgA, IgD, IgE, and IgM, may also be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fcy or the homologous domains in any of IgA, IgD, IgE, or IgM. Preferably, the Fc region comprises Pro 100 to Lys 330 of human IgG1 (see FIG. 14).

Ephrin-Fc fusions are commercially available from various sources, including Sigma-Aldrich, St. Louis, Mo., and R&D Systems, Inc. Minneapolis, Minn., which provide mouse ephrin-A1-Fc (R&D Cat. 602-A1-200), mouse ephrin-A2-Fc (R&D Cat. 603-A2-200), human ephrin-A3-Fc (R&D Cat. 359-EA-200), human ephrin-A4-Fc (R&D Cat. 369-EA-200), mouse ephrin-A4-Fc (R&D Cat. 569-A4-200), human ephrin-A5-Fc (R&D Cat. 374-EA-200), mouse ephrin-B1-Fc (R&D Cat. 473-EB-200), mouse ephrin-B2-Fc (R&D Cat. 496-EB-200), and human ephrin-B3-Fc (R&D Cat. 395-EB-200).

It is contemplated that substitution or deletion of amino acids within the immunoglobulin heavy chain constant regions may be useful in the practice of the invention. One non-limiting example includes introducing amino acid substitutions in the upper CH2 region to create an Fc variant with reduced affinity for Fc receptors (Cole et al. (1997) J. Immunol. 159:3613). Non-lytic Fc regions can be constructed to lack a high affinity Fc receptor binding site and/or a C'q1 binding site. The high affinity Fc receptor binding site can be functionally destroyed by mutating or deleting the Leu 235 of IgG1 Fc. The C'q1 binding site can be functionally destroyed by mutating or deleting Glu 318, Lys 320, and Lys 322 of IgG1 Fc. In one aspect of the invention, substitutions of alanine residues at one or more these sites can be used to render IgG1 Fc unable to direct antibody dependent cellular cytotoxicity and/or complement directed cytolysis. One of ordinary skill in the art can prepare such constructs using well known molecular biology techniques.

In various aspects of the invention, conventional recombinant DNA methodologies can be used to generate the Fc fusion proteins useful in the practice of the invention. For example, Fe fusion constructs can be generated, and the resulting DNAs can be integrated into expression vectors, and expressed to produce the fusion proteins of the invention. One example of a useful expression vector is pdCs (Lo et al. (1988) Protein Engineering 11:495, in which the transcription is directed by the enhancer/promoter of the human cytomegalovirus and the SV40 polyadenylation signal derived from nucleotides −601 to +7 of the sequence provided in Boshart et al. (1985) Cell 41:521. The vector also contains the mutant dihydrofolate reductase gene as a selection marker (Simonsen and Levinson (1983) Proc. Nat. Acad. Sci. USA 80:2495).

Further, substitution or deletion of constructs of these constant regions, in which one or more amino acid residues of the constant region domains are substituted or deleted also would be useful. For Fc fusions, expression levels often can be increased several fold by subcloning. In addition, where Fc regions are glycosylated, they can help to solubilize hydrophobic proteins. In many cases, Fc fusion proteins can be used to produce longer serum half-lives compared to ligand alone, due in part to their larger molecular sizes (see, e.g., U.S. Pat. No. 5,116,964).

For preclinical studies, non-human ephrin-Fc fusion proteins may be useful since efficacy and toxicity studies of a protein drug must be performed in animal model systems before testing in human beings. A human protein may not work in a mouse model since the protein may elicit an immune response, and/or exhibit different pharmacokinetics skewing the test results. Therefore, the equivalent mouse protein is the best surrogate for the human protein for testing in a mouse model.

An appropriate host cell can be transformed or transfected with the expression vector, and utilized for the expression and/or secretion of the target protein. Currently preferred host cells for use in the invention include immortal hybridoma cells, NS/O myeloma cells, 293 cells, Chinese hamster ovary cells, HELA cells, and COS cells. One expression system that has been used to produce high level expression of fusion proteins in mammalian cells is a DNA construct encoding, in the 5′ to 3′ direction, a secretion cassette, including a signal sequence and an immunoglobulin Fc region, and a target protein. Several target proteins have been expressed successfully in such a system and include, for example, IL2, CD26, Tat, Rev, OSF-2, DIG-H3, IgE Receptor, PSMA, and gp120. These expression constructs are disclosed in U.S. Pat. Nos. 5,541,087 and 5,726,044 to Lo et al.

Other useful fusion proteins may include, but are not limited to, a soluble ephrin (e.g., an exodomain or fragment thereof) fused to a poly-His tag, c-myc tag, E-tag, S-tag, FLAG-tag, Glu-Glu tag, HA tag, HSV-tag, V5, VSV-g, β-galalactosidase, GFP, GST, luciferase, maltose binding protein, alkaline phosphatase cellulose binding domain, or other heterologous sequences.

For some purposes, it may be desireable to include a signal sequence in an ephrin fusion protein of the invention. Signal sequences that may be used with the expression constructs of the invention include antibody light chain signal sequences, e.g., antibody 14.18 (Gillies et. al. (1989) J. Immunol. Meth. 125:191), antibody heavy chain signal sequences, e.g., the MOPC141 antibody heavy chain signal sequence (Sakano et al. (1980) Nature 286:5774), and any other signal sequences which are known in the art (see, e.g., Watson (1984) Nucleic Acids Research 12:5145). A detailed discussion of signal peptide sequences is provided by von Heijne (1986) Nucleic Acids Research 14:4683.

As would be apparent to one of skill in the art, the suitability of a particular signal sequence for use in the secretion cassette may require some routine experimentation. Such experimentation will include determining the ability of the signal sequence to direct the secretion of a fusion protein and also a determination of the optimal configuration, genomic or cDNA, of the sequence to be used in order to achieve efficient secretion of fusion proteins. Additionally, one skilled in the art is capable of creating a synthetic signal peptide following the rules presented by von Heijne, referenced above, and testing for the efficacy of such a synthetic signal sequence by routine experimentation.

In another embodiment, the ephrin fusion proteins of the invention can include a proteolytic cleavage site interposed between the secretion cassette and the target protein. A cleavage site provides for the proteolytic cleavage of the encoded fusion protein thus separating the heterologous domain (e.g., Fc region) from the ephrin sequence. Useful proteolytic cleavage sites include amino acids sequences that are recognized by proteolytic enzymes such as trypsin, plasmin, or enterokinase K. Many cleavage site/cleavage agent pairs are known (see, for example, U.S. Pat. No. 5,726,044).

Antibodies

Included in the invention are antibodies to be used as reagents, such as antibodies directed to one or more ephrins, such as ephrin-A1, A2, A3, A4, A5, B1, B2, and B3, and the corresponding receptors. Preferred are antibodies specifically directed to ephrin-A2 or B2, or the corresponding receptors. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′}, and F_{(ab′)2} fragments, and an F_{ab} expression library. In general, antibody molecules obtained from humans relates to any of the classes IgG, IgM, IgA, IgE, and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses, and types of human antibody species.

Also included as reagents are affibodies (see, e.g., U.S. Pat. No. 5,831,012), i.e., highly specific affinity proteins that can be designed to bind to any desired target molecule. These antibody mimics can be manufactured to have the desired properties (specificity and affinity), while also being highly robust to withstand a broad range of analytical conditions, including pH and elevated temperature. The specific binding properties that can be engineered into each capture protein allow it to have very high specificity and the desired affinity for a corresponding target peptide or protein. A specific target peptide or protein will thus bind only to its corresponding capture protein. The small size (only 58 amino acids), high solubility, ease of further engineering into multifunctional constructs, excellent folding and absence of cysteines, as well as a stable scaffold that can be produced in large quantities using low cost bacterial expression systems, make affibodies useful capture molecules similar to antibodies or antibody fragments, such as Fab or single chain Fv (scFv) fragments, in a variety of life science applications. In preferred aspects of the invention, an affibody is linked, conjugated, or fused to one or more affibodies to increase binding to the target molecule, or to allow binding to two or more distinct targets.

An isolated peptide or protein of the invention intended to serve as an antigen, or a portion or fragment thereof, can be used as an immunogen to generate antibodies that immunospecifically bind the antigen, using standard techniques for polyclonal and monoclonal antibody preparation. A full-length protein can be used or, alternatively, the invention provides antigenic peptide fragments of the antigen for use as immunogens. An antigenic peptide fragment comprises at least 6 amino acid residues of the amino acid sequence of the full-length protein and encompasses an epitope thereof such that an antibody raised against the peptide forms a specific immune complex with the full-length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of a soluble ephrin exodomain that is located on the surface of the peptide or protein, e.g., a hydrophilic region. A hydrophobicity analysis of the human those amino acid sequences will indicate which regions of the peptide or protein that are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. An ephrin exodomain or a fragment thereof comprises at least one antigenic epitope. An anti-ephrin antibody of the present invention is said to specifically bind to the antigen when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

Various procedures known within the art may be used for the production of polyclonal or monoclonal antibodies directed against a peptide or protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic peptide or protein, a chemically synthesized peptide or protein, or a recombinantly expressed immunogenic peptide or protein. Furthermore, the peptide or protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface-active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants that can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen that is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

Monoclonal antibodies can be prepared by any method known in the art. The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen-binding site capable of immunoreacting with a particular epitope of the antigen characterized by a unique binding affinity for it.

In one aspect, monoclonal antibodies can be prepared by hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro. The immunizing agent will typically include the protein antigen, a fragment thereof, or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103).

Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, Monoclonal Antibodies: Principles and Practice, Academic press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures, e.g., by oligonucleotide probes that are capable of binding specifically to genes encoding the heavy and light chains of murine antibodies. The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. This non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains, or fragments thereof (such as Fv, Fab, Fab′, F(ab′)2 or other antigen-binding subsequences of antibodies) that are principally comprised of the sequence of a human immunoglobulin and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539).

In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals that are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells that secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT. publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246:1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F(ab)₂ fragment produced by pepsin digestion of an antibody molecule; (ii) an F_{ab} fragment generated by reducing the disulfide bridges of an F(ab)₂ fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. For example, the bispecific antibodies of the invention can bind to more than one ephrin, such as ephrin-A1, A2, A3, A4, A5, B1, B2, and B3. Alternatively, one of the binding specificities is for an ephrin of the invention, while the second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full-length antibodies or antibody fragments (e.g. F(ab′)2 bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. In these experiments, each Fab′ fragment is separately secreted from E. coli and subjected to directed chemical coupling in vitro to form the bispecific antibody. The bispecific antibody thus formed is able to bind to cells overexpressing the target, as well as trigger the lytic activity of human cytotoxic lymphocytes against the target cells.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). In these experiments, the leucine zipper peptides from the Fos and Jun proteins can be linked to the Fab′ portions of two different antibodies by gene fusion. The antibody homodimers can be reduced at the hinge region to form monomers and then re-oxidized to form the antibody heterodimers. This method can also be utilized for the production of antibody homodimers.

The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells that express a particular antigen. These antibodies possess an antigen-binding arm and an arm that binds a cytotoxic agent or a radionuclide chelator, such as EOTUBE, DPTA, DOTA, or TETA. Another bispecific antibody of interest binds the protein antigen described herein and further binds tissue factor (TF).

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

Therapeutics

Antibodies of the invention, including polyclonal, monoclonal, humanized, and fully human antibodies, may used as therapeutic agents such as one of this invention. Such antibodies can be directed to one or more ephrin polypeptide sequences, such as ephrin-A1, A2, A3, A4, A5, B1, B2, and B3, or the corresponding receptors. Preferred are antibodies specifically directed to ephrin-A2 or B2, or the corresponding receptors. Such agents will generally be employed to treat or prevent a disease or pathology, specifically a hematopoietic disorder or a proliferative disorder of the gastrointestinal tract, reproductive tract, or skin in a subject. An antibody preparation, preferably one having high specificity and high affinity for its target antigen, is administered to the subject and will generally have an effect due to its binding with the target. Such an effect may depend on the specific nature of the interaction between the given antibody molecule and the target antigen in question. For example, administration of the antibody may abrogate or inhibit the binding of the target with an endogenous ephrin receptor to which it naturally binds.

The peptides and proteins of the invention can also be used as therapeutic agents. Such agents can comprise a soluble ephrin from one or more ephrin exodomains, or fragments thereof, such as ephrin-A1, A2, A3, A4, A5, B1, B2, and B3. Preferred are soluble ephrin-A2 and ephrin-B2. Also preferred are fusion proteins comprising soluble ephrin, e.g., ephrin-A2-Fc, and ephrin-B2-Fc. These agents will generally be employed to treat or prevent a disease or pathology, specifically a hematopoietic disorder or a proliferative disorder of the gastrointestinal tract, reproductive tract, or skin in a subject. A peptide or protein preparation, preferably one having high specificity and high affinity for its receptor, is administered to the subject and will generally have an effect due to its binding with the receptor. For example, administration of the peptide or protein may abrogate or inhibit the binding of the receptor to its endogenous ligand.

A therapeutically effective amount of a reagent of the invention relates generally to the amount needed to achieve a therapeutic objective. As noted above, this may be a binding interaction between the antibody and its target antigen that interferes with the functioning of the target, thereby promoting a physiological response. Alternatively, it may involve the binding interaction between a peptide or fusion protein and an ephrin receptor that interferes with the functioning of the receptor, thereby promoting a physiological response. The amount required to be administered will furthermore depend on the binding affinity of the therapeutic agent and the rate at which an administered agent is depleted from the free volume of the subject to which it is administered.

Diseases and Disorders

Diseases and disorders that are characterized by altered (relative to a subject not suffering from the disease or disorder) levels of cell (e.g., stem cell) proliferation may be treated with therapeutics that antagonize (i.e., reduce or inhibit) ephrin and/or ephrin receptor activity. Antagonists may be directed to ephrin-A1, A2, A3, A4, A5, B1, B2, and B3, or the corresponding receptors. Preferably, the antagonists are directed to ephrin-A2 or B2, or the corresponding receptors. Therapeutics that antagonize activity may be administered in a therapeutic or prophylactic manner. Therapeutics that may be utilized include, but are not limited to: (i) an aforementioned peptide or protein, or analog, derivatives, fragments or homologs thereof, (ii) antibodies to an aforementioned peptide or protein, or corresponding receptor; (iii) nucleic acids encoding an aforementioned peptide or protein; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989, Science 244: 1288-1292); or (v) modulators (i.e., inhibitors, agonists and antagonists, including additional peptide mimetic of the invention or antibodies specific to a peptide or protein of the invention) that alter the interaction between an aforementioned peptide and its receptor.

Diseases and disorders that are characterized by altered (relative to a subject not suffering from the disease or disorder) levels of cellular proliferation may also be treated with therapeutics that increase (i.e., are agonists to) ephrin and/or ephrin receptor activity. Therapeutics that up regulate activity may be administered in a therapeutic or prophylactic manner. Therapeutics that may be utilized include, but are not limited to, any ephrin multimer that increases Eph activation and/or signaling, or an agonist that increases bioavailability of an ephrin or ephrin receptor.

Increased or decreased levels can be detected by quantifying peptide and/or RNA levels, by, e.g., obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Therapeutic Methods

Another aspect of the invention pertains to methods of modulating the interaction between ephrins and ephrin receptors for therapeutic purposes. The modulatory method of the invention involves contacting a cell with reagent that blocks the interactions between ephrins and ephrin receptors that are associated with the cell. A reagent that modulates this activity can be, for example, a nucleic acid, peptide, fusion protein, peptidomimetic, antibody, affibody, or small molecule. In one embodiment, the reagent stimulates cell (e.g., stem cell) proliferation, for example, in the hematopoietic system. In another embodiment, the reagent inhibits cell (e.g., stem cell) proliferation, for example, in the skin, intestinal tract, or reproductive tract. These modulatory methods can be performed in vitro (e.g., by culturing the cell with the reagent) or, alternatively, in vivo (e.g., by administering the agent to a subject).

In certain embodiments of the invention, DNA constructs (or gene constructs) of the can be used as a part of a gene therapy protocol to deliver nucleic acids encoding a soluble ephrin (e.g., an exodomain or portion thereof of ephrin-A1, A2, A3, A4, A5, B1, B2, or B3) or a fusion protein construct thereof (e.g., ephrin-A2-Fc or ephrin-B2-Fc). The invention features expression vectors for in vivo transfection and expression of soluble ephrin or a fusion protein construct thereof in particular cell types. Expression constructs of a soluble ephrin, or fusion protein constructs thereof, may be administered in any biologically effective carrier, e.g. any formulation or composition capable of effectively delivering the soluble ephrin or fusion protein construct thereof to cells in vivo. Approaches include insertion of the subject gene in recombinant bacterial or eukaryotic plasmids, or viral vectors such as retroviruses. The virus may be an adenovirus, adeno-associated virus, herpes simplex virus-1, or pox virus. One preferred pox virus is vaccinia. Other viruses include iridoviruses, coronaviruses, togaviruses, caliciviruses picornaviruses, and lentiviruses. All the viruses may be from a strain that has been genetically modified or selected to be non-virulent in a host.

The methods of gene delivery and expression in a target cell may comprise (a) providing an isolated nucleic acid fragment encoding a soluble ephrin; (b) selecting a viral vector with at least one insertion site for insertion of the isolated nucleic acid fragment operably linked to a promoter capable of expression in the target cells; (c) inserting the isolated nucleic acid fragment into the insertion site, and (d) introducing the vector into the target cell wherein the soluble ephrin is expressed at detectable levels. Alternatively, the methods may comprise (a) providing an isolated nucleic acid fragment encoding an ephrin fusion protein; (b) selecting a viral vector with at least one insertion site for insertion of the isolated nucleic acid fragment operably linked to a promoter capable of expression in the target cells; (c) inserting the isolated nucleic acid fragment into the insertion site, and (d) introducing the vector into the target cell wherein the ephrin fusion protein is expressed at detectable levels.

Preferred dosages per administration of nucleic acids encoding the soluble ephrins or fusion proteins of the invention are within the range of 1 μg/m² to 100 mg/m² more preferably 20 μg/m² to 10 mg/m², and most preferably 400 μg/m² to 4 mg/m². It is contemplated that the optimal dosage and mode of administration may be determined by routine experimentation well within the level of skill in the art. Optimal dosage depends upon the disease being treated and upon the existence of side effects. However, optimal dosages may be determined using routine experimentation. Administration of the fusion protein may be by periodic bolus injections, or by continuous intravenous or intraperitoneal administration from an external reservoir (for example, from an intravenous bag) or internal (for example, from a bioerodable implant). Furthermore, it is contemplated that the nucleic acids of the invention also may be administered to the intended recipient together with a plurality of different biologically active molecules. It is contemplated, however, that the optimal combination of nucleic acids and other molecules, modes of administration, dosages may be determined by routine experimentation well within the level of skill in the art.

Determination of the Biological Effect of the Therapeutic

Also encompassed by the invention are suitable in vitro or in vivo assays that are performed to determine the effect of a specific reagent and whether its administration is indicated for treatment of the affected tissue. In specific embodiments, in vitro assays may be performed with representative stem cells or newly differentiated cells involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

The cell (e.g., stem cell or progenitor cell) referred to in this application may be a cell that is isolated from adult bone marrow, spinal cord, epithelial skin, epithelial intestinal, pancreas, hematopoietic system, blood, umbilical cord and muscle. In this embodiment, a stem cell or progenitor cell is not limited to cells only found in the system targeted for treatment. For example, a pluripotent stem cell may be isolated from any of the tissues listed and contact with the reagent may cause, directly or indirectly, the stem cell to become a hematopoietic stem cell or hematopoietic progenitor cell. As a non-limiting illustration of this concept, an embryonic stem cell can be used as a pluripotent stem cell. A pluripotent stem cell can also be isolated from body fat tissue. Thus, a stem cell or progenitor cell can be derived from any pluripotent stem cell contacted with a reagent of the invention.

Of particular interest are cells (e.g., stem cells or progenitor cells) that are derived from tissue of interest. This includes cells (e.g., stem cells or progenitor cells) obtained from bone marrow, or tissue of the skin, esophagus, stomach, small intestine, large intestine, rectum, prostate, testis, penis, ovaries, uterus, cervix, fallopian tubes, vulva, or vagina. In such cases, stem cells can be identified by their ability to undergo continuous cellular proliferation, to regenerate exact copies of themselves (self-renew), to generate a large number of regional cellular progeny, and to elaborate new cells in response to injury or disease. Such stem cells can typically generate progeny for their tissue type, and can express some of the phenotypic markers that are characteristic of their lineage. Typically, they do not produce progeny of other embryonic germ layers when cultured alone in vitro unless dedifferentiated or reprogrammed in some fashion.

Pharmaceutical Compositions

The invention provides methods of stimulating or inhibiting cells (e.g., stem cells) from producing progeny, which can be used to treat a disease, disorder, or injury, as described in detail herein. The methods of the invention can be used to treat any mammal, including humans, cows, horses, dogs, sheep, and cats. Preferably, the methods of the invention are used to treat humans. In one aspect, the invention provides a regenerative treatment for hematopoietic disorders by stimulating hematopoietic cells (e.g., stem cells) to grow, proliferate, migrate, survive, and/or differentiate to replace cells that have been lost or destroyed. In vivo stimulation of hematopoietic cells (e.g., stem cells) can be accomplished by locally administering a reagent of the invention to the cells in an appropriate formulation. By increasing hematopoiesis, damaged or missing cells can be replaced in order to enhance blood function. In other aspects, the invention provides treatments for proliferative disorders of the intestinal tract, reproductive tract, and skin, as described in detail herein.

The reagents of the invention can be formulated into pharmaceutical compositions that can be used as therapeutic agents for the treatment of diseases or disorders of the hematopoietic system, intestinal tract, reproductive tract, and skin. For example, the composition includes a reagent of the invention, which can be administered alone or in combination with the systemic or local co-administration of one or more additional agents. Such agents include preservatives, permeability increasing factors, stem cell mitogens, survival factors, lineage preventing agents, anti-apoptotic agents, anti-stress medications, protectants, and anti-pyrogenics. Preferably, the pharmaceutical composition is used to treat diseases by stimulating or inhibiting cell growth, proliferation, migration, survival and/or differentiation, and targeting the affected tissues. For treatment, a method of the invention comprises administering to the subject an effective amount of a pharmaceutical composition including a reagent (1) alone in a dosage range of 0.5 ng/kg/day to 500 ng/kg/day, (2) in a combination permeability increasing factor, or (3) in combination with a locally or systemically co-administered agent.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile, physiologically acceptable diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Oral administration refers to the administration of the formulation via the mouth through ingestion, or via any other part of the gastrointestinal system including the esophagus or through suppository administration. Parenteral administration refers to the delivery of a composition, such as a composition comprising a soluble ephrin agent by a route other than through the gastrointestinal tract (e.g., oral delivery). In particular, parenteral administration may be via intravenous, subcutaneous, intramuscular or intramedullary (i.e., intrathecal) injection. Topical administration refers to the application of a pharmaceutical agent to the external surface of the skin or the mucous membranes (including the surface membranes of the nose, lungs and mouth), such that the agent crosses the external surface of the skin or mucous membrane and enters the underlying tissues. Topical administration of a pharmaceutical agent can result in a limited distribution of the agent to the skin and surrounding tissues or, when the agent is removed from the treatment area by the bloodstream, can result in systemic distribution of the agent.

In a preferred form of topical administration, the pharmaceutical agent is delivered by transdermal delivery. Transdermal delivery refers to the diffusion of an agent across the barrier of the skin. The skin (stratum corneum and epidermis) acts as a barrier and few pharmaceutical agents are able to penetrate intact skin. In contrast, the dermis is permeable to many solutes and absorption of drugs therefor occurs more readily through skin that is abraded or otherwise stripped of the epidermis to expose the dermis. Absorption through intact skin can be enhanced by placing the active agent in an oily vehicle before application to the skin (a process known as inunction). Passive topical administration may consist of applying the active agent directly to the treatment site in combination with emollients or penetration enhancers. Another method of enhancing delivery through the skin is to increase the dosage of the pharmaceutical agent. The dosage may be increased up to ten, a hundred or a thousand folds more than the usual dosages of between 1 ng/kg/day to 50 mg/kg/day.

Pharmaceutical compositions suitable for injectable use include sterile aqueous solutions (where water soluble) or dispersions and sterile powders for the extemporaneous preparation of sterile injectable solutions or dispersion. For intravenous administration, physiologically acceptable, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Physiologically acceptable carriers maybe any carrier known in the field as suitable for pharmaceutical (i.e., topical, oral, and parenteral) application. Suitable pharmaceutical carriers and formulations are described, for example, in Remington's Pharmaceutical Sciences (19th ed.) (Genarro, ed. (1995) Mack Publishing Co., Easton, Pa.). Preferably, pharmaceutical carriers are chosen based upon the intended mode of administration of the reagent. The pharmaceutically acceptable carrier may include, for example, emollients, humectants, thickeners, silicones, and water. Suitable formulations that include pharmaceutically acceptable excipients for introducing the reagent to the bloodstream by other than injection routes can be found in Remington's Pharmaceutical Sciences (19th ed.) (Genarro, ed. (1995) Mack Publishing Co., Easton, Pa.).

Specific examples of carriers include hydrocarbon oils and waxes such as mineral oil, petrolatum, paraffin, ceresin, ozokerite, microcrystalline wax, polyethylene, and perhydrosqualene; triglyceride such as vegetable oil, animal fats, castor oil, cocoa butter, safflower oil, cottonseed oil, corn oil, olive oil, cod liver oil, almond oil, avocado oil, palm oil, sesame oil, squalene, and maleated soybean oil; acetoglycerides, such as acetylated monoglycerides; ethoxylated glycerides, such as ethoxylated glyceryl monostearate; alkyl esters of fatty acids such as methyl, isopropyl, and butyl, hexyl laurate, isohexyl laurate, isohexyl palmitate, isopropyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, lauryl lactate, myristyl lactate, and cetyl lactate esters of fatty acid; alkenyl esters of fatty acids such as oleyl myristate, oleyl stearate, and oleyl oleate; fatty acids such as pelargonic, lauric, myristic, palmitic, stearic, isostearic, hydroxystearic, oleic, linoleic, ricinoleic, arachidic, behenic, and erucic acids; fatty alcohols such as lauryl, myristyl, cetyl, hexadecyl, stearyl, isostearyl, hydroxystearyl, oleyl, ricinoleyl, behenyl, erucyl, and 2-octyl dodecanyl alcohols; fatty alcohol ethers such as lauryl, cetyl, stearyl, isostearyl, oleyl, and cholesterol alcohols, having attached thereto from 1 to 50 ethylene oxide groups or 1 to 50 propylene oxide groups; ether-esters such as fatty acid esters of ethoxylated fatty alcohols.

Also included are lanolin and derivatives such as lanolin, lanolin oil, lanolin wax, lanolin alcohols, lanolin fatty acids, isopropyl lanolate, ethoxylated lanolin, ethoxylated lanolin alcohols, ethoxylated cholesterol, propoxylated lanolin alcohols, acetylated lanolin alcohols, lanolin alcohols linoleate, lanolin alcohols ricinoleate, acetate of lanolin alcohols ricinoleate, acetate of ethoxylated alcohols-esters, hydrogenolysis of lanolin, ethoxylated hydrogenated lanolin, ethoxylated sorbitol lanolin, and liquid and semisolid lanolin absorption bases; polyhydric alcohol esters such as ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 mono-oleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid esters, sorbitan fatty acid esters, and polyoxyethylene sorbitan fatty acid esters are satisfactory polyhydric alcohol esters.

Further included are waxes such as beeswax, spermaceti, myristyl myristate, stearyl stearatepolyoxyethylene sorbitol beeswax, carnauba and candelilla waxes; phospholipids such as lecithin and derivatives; sterols such as cholesterol and cholesterol fatty acid esters, amides such as fatty acid amides, ethoxylated fatty acid amides, and solid fatty acid alkanolamides. In addition, the reagent and the pharmaceutically acceptable carrier may be enclosed in a hard or soft shell gelatin capsule, compressed into tablets, or incorporated directly into the individual's diet. Specifically, the reagent may be incorporated with excipients and used in the form of ingestible tablets, buccal tablets, troches, capsules, elixirs, suspensions, syrups, wafers, and the like. When the soluble ephrin agent is administered orally, it may be mixed with other food forms and pharmaceutically acceptable flavor enhancers. When the soluble ephrin agent is administered enterally, they may be introduced in a solid, semi-solid, suspension, or emulsion form and may be compounded with any number of well-known, pharmaceutically acceptable additives. Sustained release oral delivery systems and/or enteric coatings for orally administered dosage forms are known in the art and also contemplated.

Oral compositions generally include a physiologically acceptable, inert diluent or an edible carrier. They can be enclosed in gelatin capsules or compressed into tablets. For the purpose of oral therapeutic administration, the reagent of the invention can be incorporated with physiological excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; physiologically acceptable excipients such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Where a reagent of the invention is administered as a topical agent, the composition of the invention may optionally comprise other agents known to have a cosmetic or beneficial effect on the skin. Such agents include, for example, antioxidants, sunscreens, a pH buffer, and a combination thereof. While any antioxidant that is chemically compatible may be used, preferred antioxidants include amino acids such as glycine, histidine, tyrosine, and tryptophan; imidazoles such as urocanic acid; peptides such as D,L-carnosine, D-carnosine, L-carnosine and anserine; carotenoids; carotenes such as alpha-carotene, beta-carotene, and lycopene; lipoic acid such as dihydrolipoic acid; thiols such as aurothioglucose, propylthiouracil, thioredoxin, glutathione, cysteine, cystine, and cystamine; dilauryl thiodipropionate; distearyl thiodipropionate; thiodipropionic acid; sulphoximine compounds such as buthionine-sulphoximines, homocysteine-sulphoximine, buthionine-sulphones, penta-, hexa- and heptathionine-sulphoximine; metal chelating agents such as alpha-hydroxy-fatty acids, palmitic acid, phytic acid, lactoferrin EDTA and EGTA; alpha-hydroxy acids such as citric acid, lactic acid, and malic acid; unsaturated fatty acids such as gamma-linolenic acid, linoleic acid and oleic acid; folic acid; ubiquinone and ubiquinol.

Also included are vitamin C and derivatives such as ascorbyl palmitate, Mg ascorbyl phosphate and ascorbyl acetate; tocopherols and derivatives such as vitamin E acetate; vitamin A and derivatives such as vitamin A palmitate; coniferyl benzoate of benzoin resin; rutic acid; alpha-glycosylrutin; ferulic acid; furfurylideneglucitol; carnosine; butylhydroxytoluene; butylhydroxyanisole; nordihydroguaiac resin acid; nordihydroguaiaretic acid; trihydroxybutyrophenone; uric acid; mannose; zinc compounds such as ZnO, ZnSO₄selenium; and stilbenes. In addition the antioxidant may include derivatives such as salts, esters, ethers, peptides, lipids, nucleotides, nucleosides of said antioxidants. The derivatives may include, for example, glycosyl, N-acetyl, methyl, ethyl, propyl, amyl, butyl and lauryl, palmitoyl, oleyl, gamma.-linoleyl, cholesteryl and glyceryl esters derivatives. Further, the antioxidants may be a combination, a physical blend, of salts of one or more antioxidants. The amount of the abovementioned antioxidants (one or more compounds) in the formulations is preferably 0.001 to 30% by weight, particularly preferably 0.05-20% by weight, in particular 1-10% by weight, based on the total weight of the formulation.

Sterile injectable solutions can be prepared by incorporating the reagent of the invention (e.g., a nucleic acid, peptide, fusion protein, antibody, affibody, and the like) in the required amount in an appropriate solvent with one or a combination of ingredients enumerated above, as required, followed by filtered sterilization. Generally, dispersions are prepared by incorporating the reagent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

A number of systems that alter the delivery of injectable drugs can be used to change the pharmacodynamic and pharmacokinetic properties of therapeutic agents (see, e.g., K. Reddy, 2000, Annals of Pharmacotherapy 34:915-923). Drug delivery can be modified through a change in formulation (e.g., continuous-release products, liposomes) or an addition to the drug molecule (e.g., pegylation). Potential advantages of these drug delivery mechanisms include an increased or prolonged duration of pharmacologic activity, a decrease in adverse effects, and increased patient compliance and quality of life. Injectable continuous-release systems deliver drugs in a controlled, predetermined fashion and are particularly appropriate when it is important to avoid large fluctuations in plasma drug concentrations. Encapsulating a drug within a liposome can produce a prolonged half-life and an increased distribution to tissues with increased capillary permeability (e.g., tumors). Pegylation provides a method for modification of therapeutic peptides or proteins to minimize possible limitations (e.g., stability, half-life, immunogenicity) associated with these reagents.

In accordance with the invention, one or more ephrins (e.g., ephrin-A1, A2, A3, A4, A5, B1, B2, or B3) can be formulated with lipids or lipid vehicles (e.g., micells, liposomes, microspheres, protocells, protobionts, liposomes, coacervates, and the like) to allow formation of ephrin multimers. Similarly, ephrins can be multimerized using pegylation, cross-linking, disulfide bond formation, formation of covalent cross-links, glycosylphosphatidylinositol (GPI) anchor formation, or other established methods. The multimerized ephrins can be formulated into a pharmaceutical composition, and used to increase or enhance ephrin-Eph interactions and/or signaling. For example, ephrin multimers can be used to treat diseases or disorders characterized by increased proliferation of hematopoietic cells, e.g., leukemias and related disorders, as described herein. Alternatively, ephrin multimers can be used to treat diseases or disorders characterized by decreased proliferation of cells of the gastrointestinal tract, reproductive tract, and skin, e.g., atrophy, ulcers, and other wounds refractive to healing, as described herein.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For administration by inhalation, the reagents of the invention can be delivered in the form of an aerosol spray from pressured container or dispenser that contains a suitable propellant, e.g., a gas such as carbon dioxide, or a nebulizer. For transdermal administration, the reagents of the invention can be formulated into ointments, salves, gels, or creams as generally known in the art. The reagents can also be prepared in the form of suppositories (e.g., with conventional suppository bases such as cocoa butter and other glycerides) or retention enemas for rectal delivery.

In one embodiment, the reagents of the invention are prepared with carriers that will protect the reagent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Nucleic acid molecules encoding a proteinaceous reagent can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in a physiologically acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

In other embodiments, the reagent is administered in a composition comprising at least 90% pure reagent. The reagent can be, for example a soluble ephrin (e.g., soluble ephrin-A2 or soluble ephrin-B2), an ephrin fusion protein (e.g., ephrin-A2-Fc or ephrin-B2-Fc), an anti-ephrin or anti-Eph antibody or affibody, soluble ephrin receptor or any combination thereof. Preferably the reagent is formulated in a medium providing maximum stability and the least formulation-related side effects. In addition to the reagent, the composition of the invention will typically include one or more protein carrier, buffer, isotonic salt, and stabilizer.

Compositions that include one or more reagents of the invention can be administered in any conventional form, including in any form known in the art in which it may either pass through or by-pass the blood-brain barrier. Methods for allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477).

In some instances, the reagent can be administered by a surgical procedure implanting a catheter coupled to a pump device. The pump device can also be implanted or be extracorporally positioned. Administration of the reagent can be in intermittent pulses or as a continuous infusion. Devices for injection to discrete areas of the brain are known in the art (see, e.g., U.S. Pat. Nos. 6,042,579; 5,832,932; and 4,692,147).

Reagents, derivatives, and co-administered agents can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the agents to affect solubility or clearance of the peptide. Peptidic molecules may also be synthesized with D-amino acids to increase resistance to enzymatic degradation. In some cases, the composition can be co-administered with one or more solubilizing agents, preservatives, and permeation enhancing agents.

For example, the composition can include a preservative or a carrier such as proteins, carbohydrates, and compounds to increase the density of the pharmaceutical composition. The composition can also include isotonic salts and redox-control agents. In some embodiments, the composition administered includes the reagent and one or more agents that increase the permeability of the cells, i.e., “permeability enhancers.” Such a composition can help an injected composition penetrate deeper into the tissue. Examples of suitable permeability enhancers include, for example, liposomes, VEGF (vascular endothelial growth factor), IL-s, TNFα, polyoxyethylene, polyoxyethylene ethers of fatty acids, sorbitan monooleate, sorbitan monolaurate, polyoxyethylene monolaurate, polyoxyethylene sorbitan monolaurate, fusidic acid and derivatives thereof, EDTA, disodium EDTA, cholic acid and derivatives, deoxycholic acid, glycocholic acid, glycodeoxycholic acid, taurocholic acid, taurodeoxycholic acid, sodium cholate, sodium glycocholate, glycocholate, sodium deoxycholate, sodium taurocholate, sodium glycodeoxycholate, sodium taurodeoxycholate, chenodeoxycholic acid, urosdeoxycholic acid, saponins, glycyrrhizic acid, ammonium glycyrrhizide, decamethonium, decamethonium bromide, dodecyltrimethylammonium bromide, and dimethyl-α-cyclodextrin or other cyclodextrins.

Drug Screening

The invention also provide a method of using one or more reagents of the invention for screening for additional agents that influence cell (e.g., stem cell and progenitor cell) proliferation. In one aspect of the invention, cells (undifferentiated or differentiated) are used to screen factors that promote maturation into a particular cell type, or promote proliferation and maintenance of such cells in long-term culture. For example, candidate agents can be tested by adding them to cells in culture at varying dosages, in the presence or absence of the reagents of the invention, and then determining any changes that result, according to desirable criteria for further culture and use of the cells. Physical characteristics of the cells can be analyzed by observing cell growth and/or division with microscopy. The induction of expression of increased levels of growth, proliferation, differentiation, survival, and/or migration can be analyzed with any technique known in the art. Such techniques include RT-PCR, in situ hybridization, and ELISA.

In various aspects, the screening methods of the invention may be used to identify agents that counter the activity of the reagents of the invention, and thereby either decrease proliferation of cells (e.g., stem cells) of the hematopoietic system or increase proliferation of cells (e.g., stem cells) of the intestinal tract, reproductive tract, or skin. In other aspects, the screening methods of the invention may be used to identify agents which mimic or amplify the activity of the reagents of the invention, and thereby increase proliferation of cells (e.g., stem cells) of the hematopoietic system, or decrease proliferation of cells (e.g., stem cells) of the intestinal tract, reproductive tract, or skin.

Alternatively, endogenous agents in cells (e.g., stem cells) can be identified using RT-PCR or in situ hybridization techniques. In particular, genes that are up regulated or down regulated in these cells in the presence of one or more reagents of the invention can be identified. The regulation of such genes may indicate that they are involved in the mediation of signal transduction pathways in the regulation of Eph-ephrin function. Furthermore, by knowing the levels of expression of the these genes, and by analyzing the genetic or amino-acid sequence variations in these genes or gene products, it may be possible to diagnose disease or determine the role of cells (e.g., stem and progenitor cells) in the disease. Such analysis will provide important information for using cell-based treatments for disease.

To determine the effect of a candidate agent on stem cells, a culture derived from multipotent stem cells can be obtained from a tissue of interest or, alternatively, from a host with a particular disease or disorder affecting the tissue. The choice of culture will depend upon the particular agent being tested and the effects one wishes to achieve. Once the cells are obtained from the desired donor tissue, they can be proliferated in vitro in the presence of a proliferation-inducing reagent. The ability of various biological agents to increase, decrease, or modify in some other way the number and nature of the stem cell progeny proliferated in the presence of the reagent of the invention can be screened using known methods.

In accordance with the invention, it is possible to screen for reagents that increase the proliferative ability of cells (e.g., stem cells) that would be useful for generating large numbers of cells for transplantable purposes. In these studies, precursor cells are plated in the presence of the candidate agent, with or without a reagent of the invention, and assayed for the degree of proliferation and survival or progenitor cells. It is possible to screen for cells that have already been induced to differentiate prior to the screening. It is also possible to determine the effects of the candidate agent on the differentiation process by applying them to precursors cells prior to differentiation. Generally, the agent will be solubilized and added to the culture medium at varying concentrations to determine the effect of the agent at each dose. The culture medium may be replenished with the agent every couple of days in amounts so as to keep the concentration of the reagent somewhat constant. Changes in proliferation are observed by an increase or decrease in the number of cells that form and/or an increase or decrease in the size of the cells, which is a reflection of the rate of proliferation and is determined by the numbers of precursor cells produced.

Using these methods, it is possible to screen for potential drug side effects on prenatal and postnatal tissues by screening for the effects of the agents on stem cell and progenitor cell proliferation and on progenitor cell differentiation or the survival and function of differentiated cells. Other screening applications of this invention relate to the testing of pharmaceutical compounds for their effect on particular tissues. Screening may be done either because the compound is designed to have a pharmacological effect on a particular cell type, or because a compound designed to have effects elsewhere may have unintended side effects in other systems. The screening can be conducted using any of the precursor cells or terminally differentiated cells of the invention. Effects on cell function can be assessed using any standard assay to observe phenotype or activity of cells, such as growth, proliferation, differentiation, and survival, either in cell culture or in an appropriate animal model.

Therapeutic Uses

The invention provides a method for in vivo disruption of ephrin-Eph activity and for therapeutic administration of reagents comprising an exodomain or a fragment thereof from one or more of ephrin-A1, A2, A3, A4, A5, B1, B2, and B3, which forms a soluble ephrin. Preferred are soluble ephrin-A2 and soluble ephrin-B2. Also preferred are soluble ephrin receptors, ephrin fusion proteins (e.g., ephrin-A2-Fc and ephrin-B2-Fc), anti-ephrin antibodies, anti-Eph antibodies, and any related molecules that can interfere with ephrin-Eph interactions. This disruption can be used to treat various diseases and disorders of the gastrointestinal tract, reproductive tract, skin, and hematopoietic system, as described herein. The term “treating” in its various grammatical forms in relation to the invention refers to preventing, curing, reversing, attenuating, alleviating, minimizing, suppressing or halting at least one deleterious effects of a disease or disorder state, disease progression, disease causative agent (e.g., bacteria or viruses), injury, or other abnormal condition.

Any of the methods of the invention may be used to alleviate a symptom of a proliferative disease of the gastrointestinal tract, including disorders of the esophagus, stomach, small intestine, large intestine, or rectum. Diseases or disorders of the gastrointestinal tract include growths and polyps of the large intestine, including sessile and pedunculated polyps, as well as polyps identified as tubular adenomas, tubulovillous adenomas (villoglandular polyps), villous (papillary) adenomas (with or without adenocarcinoma), hyperplastic polyps, hamartomas, juvenile polyps, polypoid carcinomas, pseudopolyps, lipomas, and leiomyomas, and those associated with familial polyposis disorders, such as Gardner's syndrome and Peutz-Jeghers syndrome.

Other diseases or disorders include colorectal cancers, e.g., cancers of the rectum and sigmoid, especially adenocarcinomas, colorectal cancers associated with Lynch syndrome, and colorectal cancers spread by hematogenous metastasis, regional lymph node metastasis, perineural spread, and intraluminal metastasis. Further included are small intestine tumors, e.g., benign jejunal and ileal tumors, such as hemangiomas, leiomyomas, lipomas, neurofibromas, and fibromas, as well as small intestine tumors associated with hereditary hemorrhagic telangiectasia (Rendu-Osler-Weber syndrome), angiodysplasias, and arteriovenous malformations, and malignant small intestine tumors, such as adenocarcinomas, and those small intestine tumors associated with primary malignant lymphomas, carcinoid tumors, Kaposi's sarcoma, lymphocytic leukemia, non-Hodgkin's lymphoma, Hodgkin's disease, and other cancers of the gastrointestinal tract.

Further diseases or disorders include anorectal cancers, such as adenocarcinomas, squamous cloacogenic carcinomas, melanomas, lymphomas, and sarcomas, basal cell carcinomas, Bowen's disease (intradermal carcinomas), extramammary Paget's disease, cloacogenic carcinomas, malignant melanomas, and especially, epidermoid (nonkeratinizing squamous cell or basaloid) carcinomas of the anorectum. Also included are gastric cancers, such as adenocarcinomas, lymphomas (primarily in the stomach), and leiomyosarcomas. Included, in addition, are benign tumors of the esophagus, such as leiomyomas, as well as esophageal cancers, such as carcinomas, epidermoid carcinomas, adenocarcinomsa, lymphomas, leiomyosarcomas, metastatic cancers, spindle cell carcinomas, verrucous carcinomas, pseudosarcomas, mucoepidermoid carcinomas, adenosquamous carcinomas, cylindromas (adenoid cystic carcinomas), primary oat cell carcinomas, choriocarcinomas, carcinoid tumors, sarcomas, and primary malignant melanomas.

Any of the methods of the invention may be used to alleviate a symptom of a proliferative disease of the reproductive tract, including the prostate, testis, penis, ovaries, uterus, cervix, fallopian tubes, vulva, and vagina. Diseases or disorders of the reproductive tract include benign prostate hypoplasia, as well as cancers of the prostate, such as adenocarcinomas, sarcomas, squamous cell carcinomas, ductal transitional carcinomas, and undifferentiated prostate cancers. Also included are premalignant penis conditions, such as erythroplasiast, Bowen's disease, and bowenoid papulosis, and penis cancers, such as carcinomas and squamous carcinomas. Further included are testicular tumors, including seminomas, teratomas, embryonal carcinomas, endodernal sinus tumors (yolk sac tumors), and choriocarcinomas.

Diseases or disorders of the reproductive tract include, as well, endometrial hyperplasias, and endometrial cancers, such as adenocarcinomas, sarcomas, mixed mesodermal tumors, leiomyosarcomas, and endometrial stromal sarcomas. Additionally included are polycystic ovary syndrome, and ovarian tumors, such as cystadenocarcinomas, and mucinous, endometroid, transitional cell, Brenner, clear cell, and unclassified carcinomas, and germ cell and sex cord-stromal cell tumors, e.g., dysgerminomas, immature teratomas, endodermal sinus tumors, embryonal carcinomas, choriocarcinoma, and polyembryomas, granulosa-theca cell tumors and Sertoli-Leydig cell tumors. Cervical displasias and cancers are also included, such as squamous cell carcinomas, adenocarcinomas, sarcomas, and small cell neuroendocrine tumors. Included also are vulvar cancers, such as squamous cell carcinomas, melanomas, sarcomas, basal cell carcinomas, adenocarcinomas, and transitional cell, adenoid cystic, and adenosquamous carcinomas; vaginal malignancies, such as squamous cell carcinomas, primary and secondary adenocarcinomas, secondary squamous cell carcinomas, clear cell adenocarcinomas, and melanomas; and fallopian tube cancers, such as papillary serous adenocarcinomas and sarcomas. Gestational trophoblastic disease is also included.

Diseases or disorders of the skin include psoriasis, such as erythrodermic psoriasis (exfoliative psoriatic dermatitis), pustular psoriasis (e.g., von Zumbusch type and Barber's psoriasis), inflammatory skin disease, and skin cancers, such as basal cell and squamous cell carcinomas, malignant melanomas, Paget's disease of the nipple or extramammary Paget's, Kaposi's sarcoma, and tumors of adnexae, and cutaneous T-cell lymphoma (mycosis fungoides).

Diseases or disorders of the hematopoietic system include leukopenia, lymphocytopenia, neutropenia, granulocytopenia, agranulocytosis, thrombocytopenia, coagulation factor deficiencies, and anemias, such as hypoproliferative anemias, hypoplastic anemias, Fanconi's anemias, Blackfan-Diamond syndrome, and sepsis-, cancer-, chemotherapy-, and radiation-induced anemias, and acute radiation hematopoietic syndrome.

Also included are acute leukemias, such as lymphoblastic and myelogenous types, chronic leukemias (also called chronic lymphocytic leukemias or chronic lymphatic leukemia), such as lymphocytic or myelocytic types, and myelodysplastic syndromes. Additional diseases or disorders include acute radiation GI syndrome, and ulcers, e.g., gastric ulcers, duodenal ulcers, and oral ulcers, including ulcers associated with gastroesophageal reflux disease, peptic ulcer disease, corrosive esophagitis, as well as contact ulcers, i.e., unilateral or bilateral ulcers of the mucous membrane over the vocal process of the arytenoid cartilage. Also included are corneal ulcers, such as those that are associated with Staphylococcus, Pseudomonas, or Streptococcus pneumoniae, herpes simplex keratitis, neurotrophic keratitis, chronic blepharitis, conjunctivitis, trachoma, bullous keratopathy, and cicatricial pemphigoid, entropion, trichiasis, lagophthalmos, Bell's palsy, eyelid defects after trauma, and exophthalmos. Further included are skin ulcers such as pressure sores (also called bedsores, decubitus ulcers, and trophic ulcers), and chronic, nonhealing wounds (e.g., nonhealing foot ulcers), such as those associated with age, diabetes, venous stasis disease, and immobilization. Included, as well, are skin atrophy (e.g., corticosteroid-induced skin atrophy), skin erosion, and skin excoriation, as well as disorders such as discoid lupus erythematosus (cutaneous lupus erythematosus; chronic discoid lupus erythematosus).

In one aspect, the methods of the invention may be used in autologous or allogenic grafting of bone marrow, peripheral blood or cord blood hematopoietic stem cells for the purpose of hematopoietic recovery. For example, hematopoietic stem cells can be amplified in vitro from a small amount of bone marrow aspirates contacted with the reagents of the invention. The reagents can be used to improve ex vivo culturing of hematopoietic stem cells/hematopoietic progenitors/precursors, to modulate growth, expansion, survival and/or differentiation of the cells for transplantation treatment of hematological diseases or injuries. The reagents can be used alone or in combination with other factors or compounds; for stem cell mobilization or proliferation prior to harvesting, ensuring a greater yield of stem cells; or for stimulating stem cells in the recipient after transplantation. Such cells from humans, other primates, rodents and other mammals will also be useful as tools for pharmacological testing.

Administration of the reagent of the invention, in any of the methods of this disclosure, may be accompanied by administration of a permeability enhancer that is delivered before, during, or after administration of the reagent. As necessary or desired, the reagent may be admixed with a pharmaceutically acceptable carrier. For treatment of hematopoietic disorders, therapeutic agents that may be administered before, during, or after reagent administration include stem cell mitogens, survival factors, lineage preventing agents, anti-apoptotic agents, anti-stress medications, anti-pyrogenics, and a combination thereof. For treatment of gastrointestinal tract, reproductive tract, and skin disorders, other anti-proliferative agents may be added before, after, or during administration of a reagent of the invention. This includes chemotherapy agents, cancer cell-targeted monoclonal antibodies, and the like. The treatments of the invention can be used for nonhuman mammals referred to in this disclosure, including but not limited to, rats, mice, rabbits, horses, sheep, pigs and guinea pigs.

In an additional embodiment, a reagent of the invention can be administered locally, as described above, in combination with an agent administered locally or systemically. Certain reagents of the invention may be pyrogenic following IV injection (e.g., in rats; Am. J. Physiol. Regul. Integr. Comp. Physiol. 2000 278:R1275-81). Thus, in some aspects of the invention, antipyrogenic agents like cox2 inhibitors, indomethacin, salisylic acid derivatives, and other general anti-inflammatory/anti-pyrogenic compounds can be systemically or locally administered before, during, and/or after administration of the reagent of the invention. Anti-apoptotic agents including caspase inhibitors and agents useful for antisense-modulation of apoptotic enzymes and factors can be administered before, during, or after administration of the reagent of the invention. In some aspects, it may be desirable to treat a subject with anti-stress medications such as, e.g., anti-glucocorticoids (e.g., RU486) and beta-blockers, administered systemically or locally before, during, and/or after infusion of the reagent of the invention.

The target tissue (for any of the methods of this invention that refer to target tissue for administration) may be selected from the group consisting of bone marrow, skin, esophagus, stomach, small intestine, large intestine, rectum, prostate, testis, penis, ovaries, uterus, cervix, fallopian tubes, vulva, and vagina. In particular, the targeted tissue may be a region of the brain damaged by a disease or injury. One method of the invention comprises: administering the reagent to a patient, determining the concentration of the reagent in the target tissue, and then depending on the outcome of the concentration measurement, deciding whether to continue to administer the reagent. As the concentration is decreased over time, additional administration and measurements may be made. The duration of treatment and time period of administration of reagent will also vary according to the size and condition of the patient, the severity of the disease or injury, and the specific composition and method being used.

Dosages

All the methods of this disclosure that involve administration of the reagent of the invention (e.g., a soluble ephrin, such as ephrin-A2 or ephrin-B2; a fusion protein, such as ephrin-A2-Fc or ephrin-B2-Fc; antibodies or affibodies directed to an Eph or ephrin, such as ephrin-A2 or ephrin-B2) may use known routes, including those described herein. As non-limiting examples, one or more reagents may be administered orally or by injection. The term injection, throughout this application, encompasses all forms of injection known in the art and at least the more commonly described injection methods such as subcutaneous, intraperitoneal, intramuscular, intracerebroventricular, intraparenchymal, intrathecal, and intracranial injection. Where administration is by means other than injection, all known means are contemplated including administration by through the buccal, nasal, or rectal mucosa. Commonly known delivery systems include administration by peptide fusion to enhance uptake or by via micelle or liposome delivery systems.

Methods for preparing the reagent dosage forms are known, or will be apparent, to those skilled in this art. The amount of reagent to be administered will depend upon the exact size and condition of the patient, but will be at least 0.1 ng/kg/day, at least 1 ng/kg/day, at least 5 mg/kg/day, or at least 10 mg ng/kg/day in a volume of 0.001 to 10 ml. The reagent may be administered in the dosage range of 0.1 ng/kg/day to 10 mg/kg/day; preferably about to 10 mg/kg/day; more preferably about 1 ng/kg/day to 5 mg/kg/day; and in particular about 0.1 μg/kg/day to. In another method of dosage, the modulator may be administered so that a target tissue achieves a modulator concentration of 0.1 nM to 50 nM, 0.11 nM to 100 nM, or at least 1 nM, at least 50 nM, or at least 100 nM. Preferred dosages include subcutaneous administration of at least 10 mg twice a week or at least 25 mg twice a week; subcutaneous administration of at least 0.04 mg/kg/week, at least 0.08 mg/kg/week, at least 0.24 mg/kg/week, at least 36 mg/kg/week, or at least 48 mg/kg/week; subcutaneous administration of at least 22 mcg twice a week or 44 mcg twice a week; or intravenous administration of at least 3-10 mg/kg once a month. Particularly preferred dosage ranges are 0.04 mg/kg to 4 mg/kg and 0.05 mg/kg to 5 mg/kg. These dosages may be increased 10×, 100× or 1000× in trasdermal or topical applications.

Pharmaceutical compositions suitable for use in the present invention include compositions wherein the active ingredients are contained in an effective amount to achieve its intended purpose. More specifically, a therapeutically effective amount means an amount effective to optimally stimulate or suppress cell (e.g., stem cell or progenitor cell) proliferation. It will be appreciated that the unit content of active ingredient or ingredients contained in an individual dose of each dosage form need not in itself constitute an effective amount since the necessary effective amount can be reached by administration of a plurality of dosage units (such as capsules or tablets or combinations thereof). In addition, it is understood that at some dosage levels, an effective amount may not show any measurable effect until after a week, a month, three months, or six months of usage. Further, it is understood that an effective amount may lessen the rate of the natural deterioration that comes with age but not reverse the deterioration that has already occurred. Determination of the effective amounts is well within the capability of those skilled in the art, especially in light of the detailed disclosure provided herein. The specific dose level for any particular user will depend upon a variety of factors including the activity of the specific reagent employed, the age, the physical activity level, general health, and the severity of the disorder.

A therapeutically effective dose also refers to that amount necessary to achieve the desired effect without unwanted or intolerable side effects. Toxicity and therapeutic efficacy of a reagent of the invention can be determined by standard pharmaceutical procedures in cell cultures or experimental animals. Using standard methods, the dosage that shows effectiveness in about 50% of the test population, the ED₅₀, may be determined. Effectiveness may be any sign of cell (e.g., stem cell) proliferation or suppression. Similarly, the dosage that produces an undesirable side effect to 50% of the population, the SD₅₀, can be determined. Undesirable side effects include death, wounds, rashes, abnormal redness, and the like. The dose ratio between side effect and therapeutic effects can be expressed as the therapeutic index and it can be expressed as a ratio between SD₅₀/ED₅₀. Reagents with high therapeutic indexes are preferred, i.e., reagents that are effective at low dosage and which do not have undesirable side effects until very high doses. A preferred therapeutic index is greater than about 3, more preferably, the therapeutic index is greater than 10, most preferably the therapeutic index is greater than 25, such as, for example, greater than 50. Furthermore, soluble ephrin agents that do not have side effects at any dosage levels are more preferred. Finally, soluble ephrin agents that are effective at low dosages and do not have side effects at any dosage levels are most preferred. The exact formulation, route of administration and dosage can be chosen depending on the desired effect and can be made by those of skill in the art.

Dosage intervals can be determined by experimental testing. One or more reagents of the invention should be administered using a regimen which maintains cell (e.g., stem cell) proliferation at about 50% above normal, about 100% above normal, preferably about 200% above normal, more preferably about 300% above normal and most preferably about 500% above normal. Alternatively, if a reagent is used to suppress cell (e.g., stem cell) proliferation, it should be administered using a regimen which maintains cell proliferation at about 50% below normal, about 70% below normal, preferably about 80% below normal, more preferably about 90% below normal and most preferably about 95% below normal. In a preferred embodiment, the pharmaceutical composition of the invention may comprise a reagent of the invention at a concentration of between about 0.001% to about 10%, preferably between about 0.01% and about 3%, such as, for example, about 1% by weight.

Other features of the invention will become apparent in the course of the following description of exemplary embodiments that are given for illustration of the invention and are not intended to be limiting thereof. Throughout this specification, various patents, published application, GenBank DNA and protein sequences, and scientific references are cited to describe the state and content of the art. Those disclosures, in their entireties, are hereby incorporated into the present specification by reference.

### EXAMPLES

### Example 1

### Materials and Methods

Animals

Ephrin-A2 null mutant mice were obtained from J. Flanagan, Harvard Medical School, Boston. Mouse embryos (embryonic day 18) and 8 weeks old wildtype, EphB2/EphB3 double null mutant mice, and double mutants with a truncated EphB2 were kindly provided by M. Henkemeyer, University of Texas Southwestern Medical Center, Dallas. For injections and intraperitoneal pump infusion experiments, adult male C57/BL-mice were used.

Animals

For injections, pump infusion experiments and neurosphere cultures adult male C57/BL6 mice (Jackson Laboratories) were used. Ephrin-A5 null mutants were kept on a mixed C57/BL6/Sv129 background (Frisén et al., 1998). The mice were backcrossed to a pure (8 generations) C57/BL6-background. The EphA7 null mice were generated as described (Holmberg, J., Armulik, A., Senti, K., Edoff, K., Momma, S., Cassidy, R., Ciossek, T., Flanagan, J. G., and Frisén, J. (2003). Regulation of cell number in the brain by a repressor of stem cell proliferation, in preparation). EphA7 and ephrin-A2 mutant mice strains contain a mixed 129/Sv and C57/bl6 genetic background, and wild-type littermates were used as controls in all experiments. Ephrin-A5, EphA7, and ephrin-A2 mutant mice were genotyped by PCR. Animals were kept on a 12 h light/dark cycle with free access to food and water. All experiments were approved by the local ethical committee (Stockholms Norra Djurförsöksetiska Nämnd, Stockholm, Sweden).

Osmotic Pump Implantation and Injections

Adult male C57 B1/6 mice were anesthetized with 2.5% (v/v) of 2,2,2-tribromethanol (Aldrich) and 2-methyl-2-butanol (Fluka), 1:1, in distilled water (10 ml kg⁻¹, i.p.). Ephrin-A2-Fc (Catalog # 603-A2-200), IgG-Fc, ephrin-B1-Fc, ephrin-B2-Fc (Catalog # 496-EB-200), EphA7-Fc, (0.1-100 μg/ml in PBS, R&D systems) or vehicle was delivered with a subcutaneously fitted osmotic pump (Alzet 1007D, delivering 0.5 μl/h). This was connected to a canula stereotaxically inserted 0.5 mm posterior and 0.7 mm lateral to Bregma, 2 mm below the dura mater in the right lateral ventricle. For intaperitoneal pump implantation, adult male C57 B1/6 mice were anesthetized with 2.5% (v/v) of 2,2,2-tribromethanol (Aldrich) and 2-methyl-2-butanol (Fluka), 1:1, in distilled water (10 ml kg⁻¹, i.p.). Ephrin-A2-Fc, IgG-Fc, ephrin-B2-Fc, (0.1-100 μg/ml in PBS, R&D systems) or vehicle was delivered with an osmotic pump (Alzet 2001, delivering 1.0 μl/h) placed in the intraperitoneal cavity. Recombinant mouse ephrin-A2-Fc and ephrin-B2-Fc chimera (R&D Systems) were dissolved in PBS and injected intravenously in the tail at total concentrations of 1, 10, 25, 50 and 100 μg. Control studies contained PBS or Fc protein equal to 100 μg. All animals were sacrificed through cervical dislocation, CO₂ exposure or in the case of perfused animals through a lethal dose of chloral hydrate (Sigma) followed by peristaltic intracardial perfusion with PBS and paraformaldehyde.

Test Substances

Recombinant mouse ephrin-A2-Fc and ephrin-B2-Fc chimeras (R&D Systems, Minneapolis, Minn.) were dissolved in PBS and injected via an osmotic pump (Alzet) ip or injected via syringe intravenously in the tail at total amount of 1, 10, and 100 μg. Control studies used PBS or Fc protein equal to 100 μg. BrdU dissolved in 0.9% NaCl was injected at 100 mg/kg.

Primary antibodies

| Antigen | Dilution | Raised in | Reference |
|---|---|---|---|
| BrdU | 1:200 | mouse | Becton Dickinson |
| BrdU | 1:200 | mouse | DAKO |
| BrdU | 1:100 | rat | Accurate |
| BrdU | 1:100 | rat | Harlan Sera Labs |
| Dll | 1:500 | rabbit | Boekhoff-Falk G |
| Ephrin-A2 | 1:500 | rabbit | Santa Cruz |
| Ephrin-A2 | 1:500 | goat | R&D Systems |
| EphA7 | 1:100 | rabbit | Santa Cruz |
| EphA7 | 1:100 | rat | R&D systems |
| EphA7 | 1:200 | rabbit | Ciossek T |
| EphA7-T1 | 1:1000 | rabbit | Ciossek T |
| GFAP | 1:50 | rabbit | DAKO |
| GFAP | 1:500 | mouse | Sigma |
| O4, IgM | 1:20 | mouse | Boehringer |
| | | | Mannheim |
| β-III tubulin | 1:500 | mouse | BabCO |
| PCNA | 1:400 | rabbit | Oncogene |
| PCNA | 1:200 | mouse | Oncogene |
| PSA-NCAM, | 1:200 | mouse | DSHB |
| IgM | | | |
| PSA-NCAM | 1:2000 | rabbit | Ericson J. |
| NeuN | 1:500 | mouse | Chemicon |
| S100β | 1:500 | mouse | Sigma |
| Phospotyrosine | 1:1000 | mouse | Upstate |
| | | | Biotechnology |

Expression Profiling

The bone marrow from 11 adult C57/BL male mice was obtained and processed for FACS sorting according to established protocols. To isolate the Lin⁻ population, cells were processed with a lineage depletion kit from Miltenyi Biotec. C-kit and Sca-1 staining was performed using antibodies from Becton & Dickson. Cells were FACS-sorted with a DIVA-FACS device. RNA was isolated using RNeasy mini reagents from Qiagen. The cDNA was reversed transcribed after DNase treatment of the RNA utilizing superscript II (Invitrogen). As negative controls, samples without reverse transcription were included for all Eph and ephrin genes.

BrdU Labeling

The thymidine analog, bromodeoxyuridine (BrdU), was used to visualize cells in S-phase. Following injection 2 h or 20 h prior to sacrifice, the animals were anesthetized with chloralhydrate and perfused with 4% buffered paraformaldehyde (PFA). Samples were postfixed in 1% PFA in phosphate buffered saline (PBS, 0.1M, pH 7.4) overnight at 4° C. The upper part of the small intestine was localized and removed together with a piece of skin from the back. The skin was then shaved with a scalpel before further processing for immunohistochemistry.

Processing of Bone Marrow and Blood

Approximately 200 μl blood per mouse was obtained through the tail vein. Whole blood counts were performed by Sveriges Lantbruksuniversitet in Uppsala (SLU). Following anesthetization, the mice were intracardially perfused with PBS. Hindlimbs were removed and the bone marrow obtained and processed according to standard protocols. To measure the Fc-protein content in blood of injected mice, a human IgG ELISA-kit from ZeptoMetrix was used.

Immunohistochemistry

The isolated tissues were cryoprotected in 20% sucrose in PBS overnight at 4° C. and mounted in Tissue tek® (Sakura Finetek USA Inc., Torrance, Calif.). Transverse 12 μm sections were cut on a cryostat, collected on precoated Superfrost® slides (VWR Scientific, West Chester, Pa.), and processed either immediately or after storing at −80° C. After rinsing in PBS, sections were processed to visualize nuclei immunoreactive with BrdU and/or PCNA, as follows. Slides with sections were treated with 2 M HCl and 0.5% Triton X-100 (Sigma Chemical Co, St Louis, Mo.) for 30 min at 37° C. This was followed by blocking with 5% bovine serum albumin, BSA (w/v) in PBS for 1 h at room temperature.

The sections were incubated overnight at 4° C. with rat anti-BrdU (BD Immunocytometry Systems, San Jose, Calif.) and/or rabbit anti-PCNA (Oncogene, San Diego, Calif.) diluted to a concentration of 1:200 and 1:400 respectively, in PBS containing 2% BSA. Following rinsing in PBS, the sections were incubated with anti-rabbit antibodies conjugated with Cy3 conjugated and/or anti-rat antibodies conjugated with Alexa 488 (Molecular probes, Eugene, Oreg.) at concentrations of 1:1000 in PBS for 1 hr in darkness at room temperature.

To visualize the total number of nuclei, the sections were rinsed in PBS and then exposed to a solution of bisbenzimide (Sigma Chemical Co, St Louis, Mo.) to a final concentration of 20 μg/ml PBS during 10 min at room temperature in darkness. Following rinsing in PBS, the sections were mounted with vectashield mounting medium (Vector, Burlingame, Calif.). To detect Fc-chimeras, sections from injected animals were incubated with either Alexa488-conjugated goat anti-human antibodies at 1:500 or Cy3-conjugated donkey anti-human antibodies at 1:500. For all immunohistochemistry, control studies included exclusion of the primary antibody, which resulted in the absence of immunoreactivity.

Photography

The preparations were evaluated and photographed on a Zeiss Axioplan microscope equipped with fluorescence, and a digital camera connected to a PC computer. The digital images were processed using the software Openlab and Adobe Photoshop 7.0. Six images obtained from 3 sections per animal were collected and processed for cell counting.

Statistics

Analysis of variance (ANOVA), Scheffe F-test and students T-test was used to calculate significant differences of the mean values between ephrin-treated or knockout preparations and their control/wildtype with respect to total cell number and BrdU and PCNA labeled cells.

### Example 2

### Results

Eph-ephrin Expression in the Bone Marrow and Hematopoietic System of Adult Mice

In previous microarray analyses, three Eph and three ephrin genes were shown to be enriched in HSC-populations: EphA3, A5, B6 and ephrin-A1, -A3, -B2 (Ivanova et al., 2002; Ramalho-Santos et al., 2002). To further resolve the expression of ephrins and Eph recptors in different compartments of the bone marrow and at different stages in the hematopoietic lineage, the expression of mRNAs encoding ephrins and Eph receptors was analyzed by RT-PCR in whole bone marrow, peripheral blood and cell surface marker identified stem cells (Lin⁻/Sca-1⁺/c-Kit⁺) and differentiated cells (Lin⁺) in bone marrow isolated by fluorescence activated cell sorting (FACS) (Table 1).

TABLE 1

| | Lin−/Sca+/c-kit+ | Lin+ | BM | WB | gut | skin |
|---|---|---|---|---|---|---|
| ephrin-A1 | − | − | − | + | (+) | − |
| ephrin-A2 | − | + | + | + | + | (+) |
| ephrin-A3 | − | − | − | − | + | + |
| ephrin-A4 | + | − | + | + | − | − |
| ephrin-A5 | − | − | − | − | + | + |
| ephrin-B1 | − | − | − | − | + | − |
| ephrin-B2 | + | − | + | + | + | + |
| ephrin-B3 | + | + | + | + | − | − |
| EphA1 | + | (+) | + | + | − | − |
| EphA2 | − | − | + | + | + | (+) |
| EphA3 | − | − | + | + | − | − |
| EphA4 | − | − | − | + | − | (+) |
| EphA5 | + | − | − | − | − | − |
| EphA6 | − | − | − | − | − | − |
| EphA7 FL | + | − | + | (+) | + | − |
| EphA7 T1 | + | − | + | − | − | − |
| EphA7 T2 | + | − | + | − | − | − |
| EphA8 | − | − | (+) | + | − | − |
| EphB1 | − | − | − | − | − | − |
| EphB2 | − | − | − | − | (+) | − |
| EphB3 | − | − | − | + | + | − |
| EphB4 | + | − | − | − | + | (+) |
| EphB6 | + | − | + | + | + | + |

Profile of expression in different compartments of the blood system: Lin⁻/Sca-1⁺/c-kit⁺ cells, Lin⁺ unsorted cells, bone marrow (BM) and whole blood (WB). The symbol “+” means detectable expression through RT-PCR; the symbol “−” means undetectable levels of expression. Symbols in parenthesis represent weak expression.

The results of these experiments indicated that receptors of both A- and B-type were selectively expressed in the Lin⁻/Sca-1⁺/c-Kit⁺ population compared to the Lin⁺ population. The ligand ephrin-B3 was expressed in all analyzed populations whereas ephrin-A4 and ephrin-B2, which were readily detected in the Lin⁻/Sca-1⁺/c-Kit⁺ group, did not appear in the Lin⁺ population. Ephrin-A2 on the other hand was not expressed in the Lin⁻/Sca-1⁺/c-Kit⁺ group, but appeared in the Lin⁺ group. The weaker or absent expression in whole bone marrow and whole blood in comparison to the sorted Lin⁻/Sca-1⁺/c-Kit⁺ cells was attributed to the enrichment of Eph/ephrin expressing cells in the sorted population.

Administration of Ephrin-A2-Fc and/or Ephrin-B2-Fc Fusion Proteins Increase BrdU-Incorporation in the Bone Marrow and in Hematopoietic Stem Cells

The expression of EphA and B receptors in Lin⁻/Sca-1⁺/c-Kit⁺ cells prompted us to investigate whether ephrins may regulate proliferation in this system. Utilizing the promiscuity of Eph-ephrin interactions soluble ephrin-A2-Fc or ephrin-B2-Fc was delivered into the circulatory system of adult mice. Because these fusion proteins are capable of binding to all the receptors within their own class, they can be used to block a large proportion of the receptors from binding to endogenous ligands. In addition, since unclustered soluble ephrins fail to activate Eph receptors, and instead act as antagonists (Davis et al., 1994), the fusions can be used to create a pan-ephrin null condition while they are in circulation. Moreover, while the receptors are occupied by ephrin-Fc proteins, the fusions can inhibit Eph forward signaling and reverse signaling through ephrins.

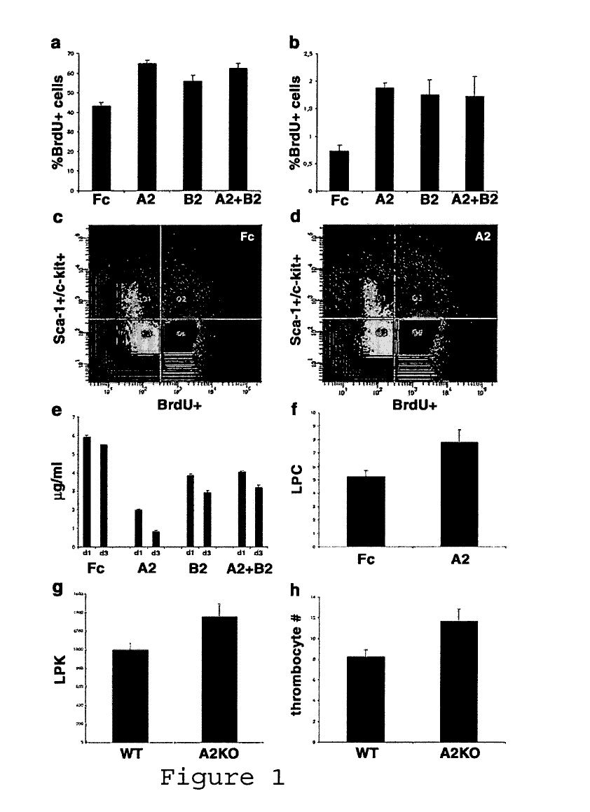

Adult mice received a single intravenous injection of recombinant protein and were given an injection of the thymidine analog bromodeoxyurine (BrdU) 3 days later. After 24 h, BrdU incorporation in bone marrow was analyzed. Also analyzed were marker-identified cell populations. BrdU incorporation was significantly increased in whole bone marrow in both ephrin-A2-Fc and ephrin-B2-Fc injected animals (FIG. 1A). The population enriched with Sca-1+/c-kit+stem cells showed an even greater increase in BrdU-incorporation where ephrin-Fc (A2 or B2) was injected, as compared to animals that had received and injection of Fc alone (FIG. 1B-D).

However, combined delivery of ephrin-A2-Fc and ephrin-B2-Fc failed to increase the BrdU-incorporation above the levels seen for the single injections in whole bone marrow and the Sca-1+/c-Kit⁺-population (FIG. 1A-B). Analysis of the concentration of recombinant protein in serum one day after injection (d1) and on the day of sacrifice three days later (d3) reveled stable levels in all groups, although, it appeared that ephrin-A2-Fc was cleared faster from the system than the other proteins (FIG. 1E).

Injection of Ephrin-A2-Fc Increases the Number of Differentiated Cells in Peripheral Blood

An increase in proliferation in the HSCs could have two major outcomes in the downstream differentiated populations. Either increased mitotic activity would be counterbalanced by increased apoptosis or decreased proliferation at other stages in the lineage, and the final output in fully differentiated cells would be unchanged. Alternatively, one or more of the lineages would produce a higher number of differentiated cells. Counts of erythrocytes, leukocytes, and platelets in ephrin-A2-Fc injected animals revealed that at least one lineage had a higher number of differentiated cells. The leukocyte particle counts were elevated in ephrin-A2-Fc injected animals compared to control animals receiving Fc protein (FIG. 1F). The concentration of erythrocytes and thrombocytes was not significantly altered by ephrin-Fc at this time point. It was hypothesized that the physiologically rapid turnover rate of leukocytes could contribute to the rapid response in this lineage, whereas the slower kinetics in the other lineages could delay alterations in the concentrations of other differentiated cells.

Ephrin-A2 Null Mutant Mice Exhibit Increased Numbers of Blood Cells

The increased proliferation of HSC and increase in leukocytes in animals that received an intravenous injection of ephrin-A2-Fc suggested that ephrins and Eph receptors may act as negative regulators of hematopoiesis. To further test this, ephrin-A2 null mice were analyzed. Ephrin-A2 mRNA expression was detected in Lin⁺ cells and whole bone marrow (Table 1), suggesting that ephrin-A2 may be a physiological ligand for EphA receptors in hematopoietic stem cells. Analysis of the blood profile of ephrin-A2 null mice revealed a significant increase in cells belonging to two distinct lineages: leukocytes and thrombocytes (FIG. 1G-H). The average concentration of erythrocytes in the mutant mice was higher compared to their wild-type littermates, although not statistically significant from the number of animals analyzed. Taken together, results from injections of ephrin-Fc protein and analysis of ephrin-A2 null mice suggest that ephrins negatively govern proliferation of hematopoietic stem cells and that this controls the number of at least certain differentiated cells in this lineage.

Disruption of Endogenous Eph-Ephrin Interactions Affects Proliferation in the Stem Cell Niche of the Small Intestine

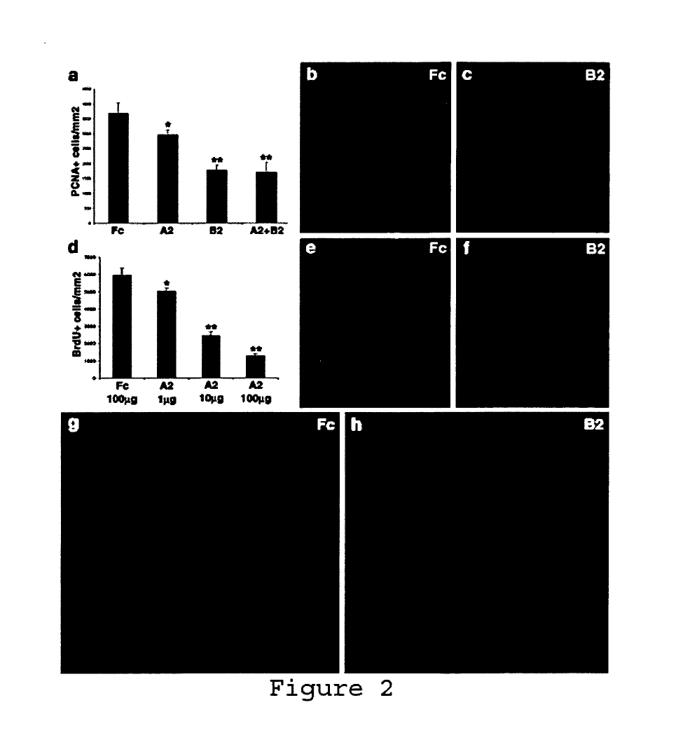

Stem cells of the small intestine reside in the crypts of Lieberkühn. Recent reports highlight the importance of EphB-ephrin-B interactions in orchestrating proper migration of stem cell progeny in this system (Batlle et al., 2002). Several members of the ephrin and Eph family are expressed in the whole small intestine (Table 1). Cell proliferation was analyzed in the crypts of ephrin-A2-Fc, ephrin-B2-Fc and combined A2/B2 injected animals. Surprisingly, injection of ephrin-A2-Fc, ephrin-B2-Fc, and combined A2/B2 was associated with a clear decline in proliferation in this compartment compared to control animals that received an injection of Fc alone (FIG. 2A-D). This decline was confirmed with counting both BrdU- and PCNA-positive cells. The injected ephrin-B2-Fc proteins were readily detected on the cell surface of the EphB expressing cells residing in the crypts with an antibody against human Fc (FIG. 2G-H).

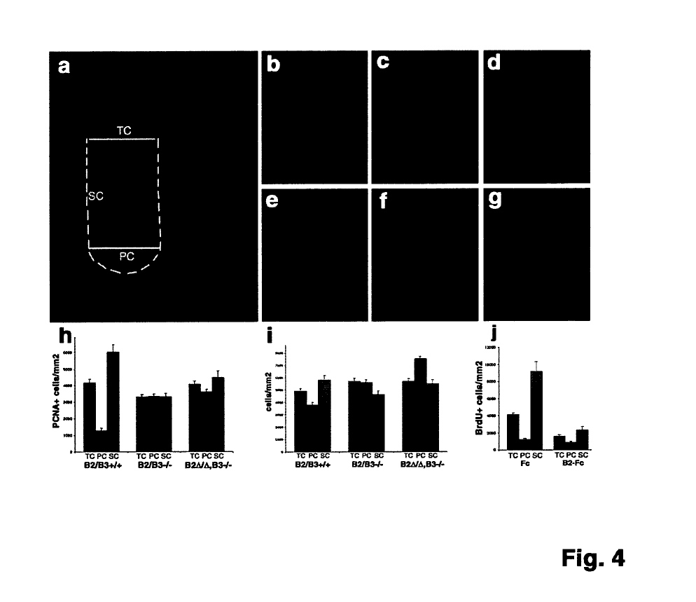

These results further demonstrated the efficiency of the injected fusion proteins in disrupting endogenous ephrin-Eph interactions. As the animals were sacrificed only three days after injection, the effects of the proteins ought to be primarily anti-proliferative and not due to a migration defect. The distribution of Paneth cells was not significantly disrupted in the ephrin-Fc (A2 or B2) injected animals (FIG. 2E-F), indicating that the decrease in cell proliferation was not a secondary effect to disrupted cell migration. To further to dissociate between direct effects on proliferation and secondary effects due to disturbed cell migration, the effect on cell proliferation was analyzed after only 24 hours exposure to ephrin-B2-Fc. Attenuation of proliferation, but no mispositioning of cells, was observed in ephrin-B2-Fc injected animals, arguing for a primary effect on proliferation (FIG. 4J).

EphB2/B3 Null Mutant Embryos Suffer an Attenuation of Proliferation in the Small Intestine

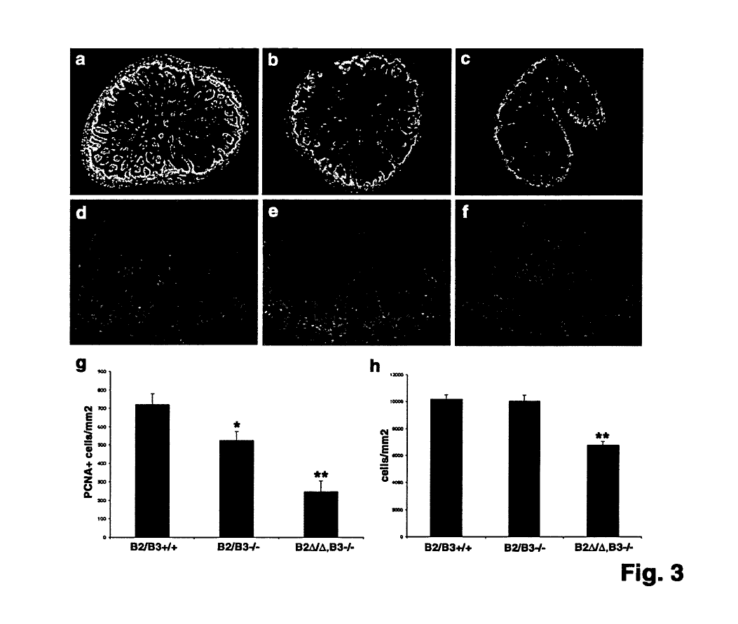

In embryonic day 18 embryos, the multipotent rapidly proliferating cells of the small intestine are confined to the intervillus pockets (Marshman et al., 2002). EphB2 and EphB3 expression is restricted to this compartment of the developing intestine whereas the ligand ephrin-B1 shows an complementary pattern of expression in the epithelial cells above these proliferative pockets (Batlle et al., 2002). Recent data has shown that the proliferating and differentiated cells intermingle in the EphB2/B3 null mutant due to lack of repulsive cues between the populations (Batlle et al., 2002). The total number of proliferating cells was counted in this compartment. It was observed that the progenitor cells go astray in the double null mutant, and they are significantly reduced in the mutant compared to wild-type (FIG. 3A-G).

The same observations were made for mice that lack EphB3 and express a mutant form of EphB2 with only the extracellular part intact and the intracellular kinase domain replaced by β-galactosidase (EphB2Δ/Δ). The chimeric EphB2Δ receptor can still bind ephrin-Bs and activate reverse signaling, whereas EphB2 forward signaling is abolished (Henkemeyer et al., 1996). Thus, analysis of these mutant mice can be used to distinguish between the roles of forward and reverse signaling. In addition, the EphB2Δ receptor has been shown to act as a dominant negative inhibitor for other EphB receptors. In these experiments, the total number of cells as determined by nuclei counts was found to be unchanged between the EphB2/B3 null mutant and wild type mice (FIG. 3A-H). However, the EphB3/EphB2Δ/Δ mutant mice displayed fewer cells in the developing intestine (FIG. 3A-H). The more severe phenotype in EphB3/EphB2Δ/Δ mutant mice compared to EphB2/B3 null mutant mice was attributed to the expression of EphB4 (Stephenson et al., 2001) in the same cells, since the signaling of this receptor is inhibited by the dominant negative effects of the EphB2Δ protein. These data suggested that EphB forward signaling positively regulated cell proliferation of the stem cells in the small intestine.

Aberrant Cell Positioning and Proliferation in the Crypts of Lieberkühn of adult EphB2/B3 Mutant Mice

The small intestine develops in a process of increasing complexity. The intestinal stem cells reside in the crypts of Lieberkühn, in deep pockets corresponding to the intervillus epithelium in the embryo. The stem cells in the adult give rise to four distinct cells types: absorptive cells (which are the most numerous), Paneth, enteroendocrine, and goblet cells. Paneth cells reside in the very bottom of the crypts, whereas the stem cells are located in the position just above these cells. Cell proliferation is most abundant in the stem/progenitor cell compartment above the Paneth cells, and newborn cells migrate, depending on their fate, up- or downwards (Marshman et al., 2002). Mice lacking EphB2/B3 receptors exhibited a modest overall decrease in proliferation in the crypts (FIG. 4H). If the Paneth cell compartment and the stem/progenitor cell population on the side of the crypt (SC) were considered separately, a shift in the number of proliferative cells from SC to PC was clearly discernable in the double null mutant mice. The same shift was seen in the EphB2Δ/Δ/EpbB3-/-mice. No significant differences in the total number of cells were detectable in the whole crypts but a shift of cells from SC to PC was seen in the mutants. (FIG. 4H-I).

Proliferation in a Stem Cell Niche in the Skin is Decreased Following Injection of Ephrin-A2-Fc or Ephrin-B2-Fc

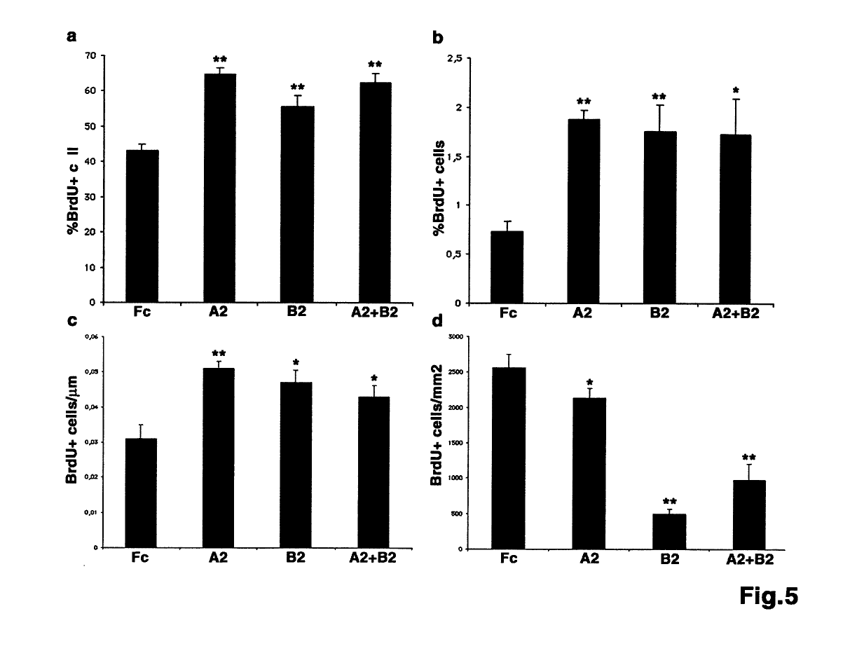

To investigate whether other stem cells could be affected by the ephrin-Fc injections, skin cells were tested. The stem cells of the skin can be found in the bulge region of hair follicles. In line with the effect we found in gut, the proliferation of these progenitors was dramatically reduced in ephrin-A2-Fc and in ephrin-B2-Fc injected animals. (FIG. 5A-B). Expression levels are shown in Table 1.

A and B Class Ephrins do not Have Additive Effects in Stem Cell Niches

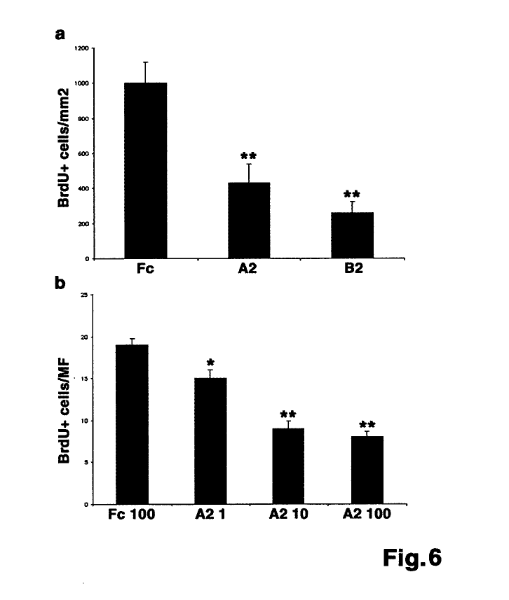

Despite the promiscuity of binding exhibited within the ephrin classes only the EphA4 receptor has been shown to bind ephrins of both B and A class. This suggested that the effect of ephrin administration on cell proliferation could be increased if a combination of ephrin-A2-Fc and ephrin-B2-Fc was injected. However, this was not observed in any system analyzed, including brain (Holmberg), blood, and intestine. In brain and blood cells, the infusion of ephrin-As had a slightly stronger effect than ephrin-Bs in increasing proliferation (FIG. 6A-C). The negative effect on proliferation seen in the small intestine was most profound in the ephrin-B injected animals (FIG. 6D). The combined infusions had no synergistic or additive effect.

### Example 3

### Discussion

Research encompassing the function of Eph receptors has primarily dealt with the developing organism and often focused on CNS development. There has been scarce research published on Eph function in stem cells and progenitor pools. Yet, it has been established that Eph receptors and ephrins are expressed in many well known stem cell populations, both in vertebrates and in other phyla (Imai, 2003; Miller et al., 2003). In the adult brain, both A- and B-class ephrins have been shown to regulate the proliferation of stem cells residing in the lateral wall of the ventricular system (Conover et al., 2000).

Eph-Ephrin Interactions Negatively Regulate Hematopoiesis

The data shown herein indicates that ephrins and Eph receptors are involved in hematopoiesis. As is the case for the adult neural stem cell population, it appears that the Eph receptors themselves are expressed in the most primitive compartment whereas the ephrin ligands appear on progeny migrating away (Table 1). This is the same for stem cells in the intestinal epithelium (Batlle et al., 2002). As shown herein, infusion of soluble ephrin fusion proteins released the inhibition of proliferation imposed on the cells by the endogenous Eph-ephrin interactions. The numbers of thrombocytes and leukocytes was found to be significantly higher in the ephrin-A2 null mutant. As the closest common progenitor for these cells belong to the multipotent stem cells of the bone marrow, this data fits with the hypothesis that the disruption of endogenous ephrin signaling in stem or progenitor cells allows for a higher rate of proliferation and production of progeny.

Without wishing to be bound by theory, the results suggest a feed-back mechanism in the hematopoietic lineage, where the presence of ligand expressing progenitor or differentiated cells may inhibit proliferation of stem cells. In this mechanism, a drop in the number of progenitor or differentiated cells may lift the suppression of proliferation of the stem cell population, and result in an increase in the production of progeny. In an alternative model, receptor-expressing stem cells may inhibit ligand-expressing progenitor cells through the ligand, and thereby suppress proliferation. The regulation of the hematopoietic stem cell population is poorly characterized, but it also possible that non-hematopoietic cells such as stromal cells play a crucial role in regulating many aspects within the population. In addition, bone marrow stromal cells express members of the ephrin and Eph families, including ephrin-A2 (Hackney et al., 2002), and it is therefore plausible that at least part of the effect on hematopoiesis of blocking ephrin-Eph interaction may be due to altered communication between hematopoietic and non-hematopoietic cells in the bone marrow.

Ephrins Regulate Cell Positioning and Proliferation in the Small Intestine

Several lines of evidence have established members of the Wnt family as key regulators of stem cell proliferation in the intestine. Transgenic overexpression of dickkoppfl, a Wnt binding protein, drastically reduces proliferation and results in atrophy of the epithelium (Pinto et al., 2003). Disruption of components of the Wnt signal transduction pathway such as Tcf-4 gives similar results (Korinek et al., 1998). Several Wnts are expressed in the intestine, and the expression is believed to be highest in cells subjacent to the intervillus epithelium in the embryo and surrounding the bottom of the crypt in the adult. This results in a gradient of mitogen with lower levels higher up in the epithelium (van de Wetering et al., 2002). Wnt signaling is mirrored by nuclear-localized β-catenin in cells close to the Wnt source (van de Wetering et al., 2002).

β-catenin is a positive regulator of c-myc which drives proliferation, in part by directly inhibiting expression of p21 (van de Wetering et al., 2002). In addition, β-catenin is a positive regulator of EphB expression, and β-catenin mutant mice fail to express EphB receptors (Batlle et al., 2002). EphB receptors are necessary for correct cell positioning in the intestinal epithelium (Batlle et al., 2002). As shown herein, reduced EphB signaling in the embryonic small intestine results in reduced cell proliferation. At this developmental stage, the composition of cells in the epithelium appears to be uniform, indicating that the reduced proliferation may be a direct effect rather that a secondary consequence of altered cell positioning.

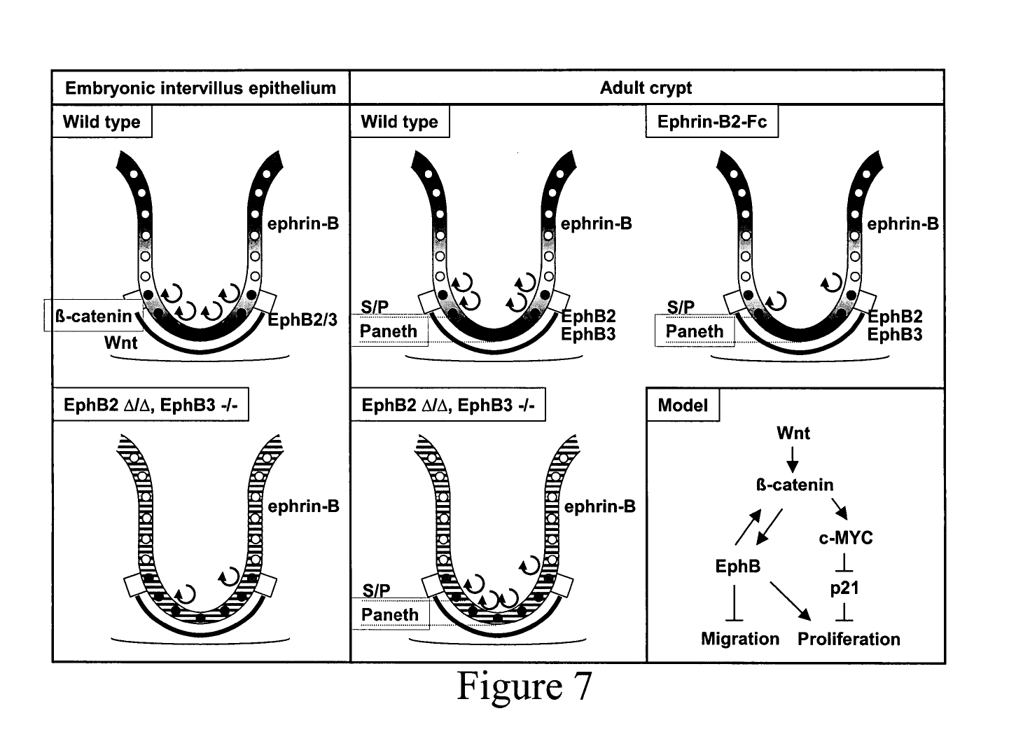

In adults, the structure of the intestine is more complex. EphB3 is required for the correct positioning of Paneth cells in the bottom of the crypt, and in the absence of this receptor the Paneth cells are scattered throughout the crypt. Paneth cells are postmitotic and very little proliferation is therefore normally seen in the bottom of the crypts. As described herein, the EphB2/EphB3 mutants showed Paneth cells to be displaced by other cells (which were not post-mitotic) at bottom of the crypt. In addition, increased proliferation in this compartment was observed in EphB mutant mice. This was attributed to the high concentration of the mitogen Wnt at the bottom of the crypt. Without wishing to be bound by theory, it is possible that the mispositioning of Paneth cells results in the exposure of more cells to Wnt. However, cell proliferation is reduced in EphB mutant mice in the compartment above where the Paneth cells normally are located. This suggests that, except for increased exposure of cells to mitogen at the bottom of the crypt, proliferation is reduced in EphB mutant mice. From this, it can be concluded that EphB signaling positively regulates proliferation of intestinal stem or progenitor cells (FIG. 7).

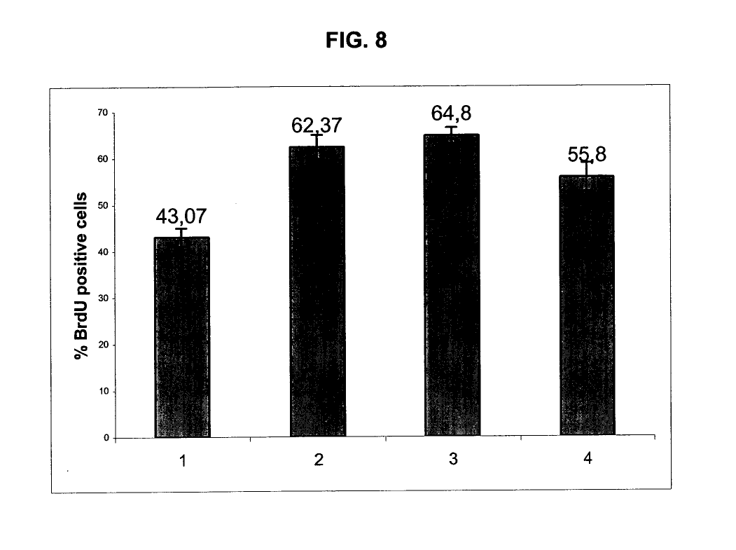

To further dissociate between EphB signaling in cell positioning and proliferation, BrdU incorporation and PCNA labeling was analyzed in animals that had been given a single injection of ephrin-B-Fc proteins. This resulted in acute inhibition of EphB signaling, but only very limited mispositioning could be seen in the intestines. The pronounced reduction in cell proliferation strongly suggested that EphB signaling acts as an important positive regulator of proliferation in the intestine (FIG. 7). Because EphB expression is regulated by β-catenin signaling, the proliferative effect of this pathway may be imposed by both c-myc and EphB (FIG. 8). It can be postulated that high EphB expression will position the cell towards the bottom of the crypt to ensure continued high exposure to Wnt, and EphB expression can thereby indirectly regulate β-catenin expression (FIG. 7, red arrow).

Common Effects of Ephrins in Stem Cell Niches

Disruption of ephrin-Eph interactions have been shown herein to have clear effects on cell proliferation in stem cell niches in the brain, bone marrow, intestine, and skin. In all these tissues, infusing proteins inhibiting either the A- or B-class ephrins have parallel effects in each tissue, i.e. if one class inhibits proliferation, the other class does likewise, and if one class stimulates proliferation, a similar effect is seen with the other class. Another common finding is that blocking A- and B-class ephrin-Eph interactions simultaneously, by administering ephrin-A2-Fc and ephrin-B2-Fc together, does not result in an additive or synergistic effect. There are several potential explanations for this. Without being bound by theory, it is possible that both A- and B-class ephrins and Eph receptors act on the same pathway, and that this pathway may be rate limiting. In this scenario, inhibiting either the A- or B-class may lower the ephrin influence on this pathway maximally and further inhibition of the other class will not have any additional result. Another possibility is that there may be co-operativity between the A- and B-classes. For example, one class could regulate the expression of the other class. Alternatively, there may be A/B ephrin or Eph heterodimers in vivo, and blocking one class could then result in inhibition of the other class.

Differential Effects of Ephrins in Stem Cell Niches

An unexpected finding in this experiments described herein is that ephrins have opposite effects on cell proliferation in different tissues. Ephrins act as negative regulators in the brain and hematopoietic systems, and positive regulators in the intestine and skin systems. Ephrins and Eph receptors are best known for their roles in regulating axon and cell migration, and few studies have found effects on cell proliferation. Most other tyrosine kinase receptors have important mitogenic functions and can act as oncogenes. However, Eph receptors are clearly different in this respect, since constitutively active receptors fail to transform cells (Lhotak and Pawson, 1993). The opposing effects of ephrins in different tissues could potentially prove helpful in the analysis of the corresponding signal transduction pathways in stem cell populations.

There is at least one other signaling pathway that has opposing effects on cell proliferation in different stem cell populations. Notch signaling positively regulates cell proliferation in the hematopoietic system, where constitutively active mutant forms of Notch can cause leukemia (Screpanti et al., 2003). Notch signaling is also a positive regulator of neural stem cell proliferation (unpublished data). In contrast, Notch inhibits cell proliferation in the skin where it acts as a tumor suppressor (Nicolas et al., 2003). Thus, both the ephrin/Eph and Notch pathways have differential effects on cell proliferation in different stem cell populations and the outcome of the signal is cell context dependent. Characterization at the molecular level how ephrins and Eph receptors regulate stem cell proliferation will be important for our understanding of stem cell biology. The potent effects of single injections of ephrin-Fc proteins suggest that manipulating this pathway may be attractive in regenerative medicine.

### Example 4

As shown in Table 2, intraperitoneal administration of recombinant mouse ephrin-A2-Fc or ephrin-B2-Fc or a combination of ephrin-A2 and ephrin-B2 was found to significantly increase the number of newborn cells (BrdU positive cells) in mouse bone marrow. The percentage of hematopoietic stem cells (identified as Sca1 and c-Kit positive, Thy-1 low) labelled with BrdU (marker for newborn cells) was significantly increased, indicating an ephrin-induced increase in symmetric stem cell division, leading to an increased pool of hematopoietic stem cells in the bone marrow.

TABLE 2

| | % BrdU+ cells in | | | % Sca1+/c-Kit+/Thy-1lo | | |
|---|---|---|---|---|---|---|
| Animals | bone marrow | SEM | p-value | & BrdU+ in bone marrow | SEM | p-value |
| Fc 1 | 40.1 | | | 0.7 | | |
| Fc 2 | 44 | | | 0.9 | | |
| Fc 3 | 45.1 | | | 0.6 | | |
| | 43.06666667 | 1.97777778 | | 0.733333333 | 0.11111111 | |
| A/B 1 | 66.2 | | | 2 | | |
| A/B 2 | 62.2 | | | 2 | | |
| A/B 3 | 58.7 | | | 1.2 | | |
| | 62.36666667 | 2.55555556 | 0.000937621 | 1.733333333 | 0.35555556 | 0.011789372 |
| A1 | 64.7 | | | 2 | | |
| A2 | 63.9 | | | 1.7 | | |
| A3 | 68.2 | | | 1.9 | | |
| A4 | 62.4 | | | 1.9 | | |
| | 64.8 | 1.7 | 4.83103E−05 | 1.875 | 0.0875 | 5.66559E−05 |
| B1 | 55.6 | | | 2.4 | | |
| B2 | 58.4 | | | 1.5 | | |
| B3 | 57.9 | | | 1.7 | | |
| B4 | 58.9 | | | 1.4 | | |
| B5 | 48.5 | | | 1.8 | | |
| | 55.86 | 3.048 | 0.001891484 | 1.76 | 0.272 | 0.002710905 |

“SEM” indicates standard error of mean.

Mice were exposed to (1) recombinant human IgG-Fc protein; (2) combination of recombinant mouse ephrin-A2 and recombinant mouse ephrin-B2; (3) recombinant mouse ephrin-A2; or (4) recombinant mouse ephrin-B2 through i.p. administration for 24 h. Animals were given BrdU and sacrificed 24 h later as described above. BrdU positive cells were quantified by FACS analysis. The data indicated that administration of recombinant mouse ephrin-A2-Fc, ephrin-B2-Fc, alone or combined, significantly increased the number of newborn cells (BrdU positive) in mouse bone marrow (FIG. 8).

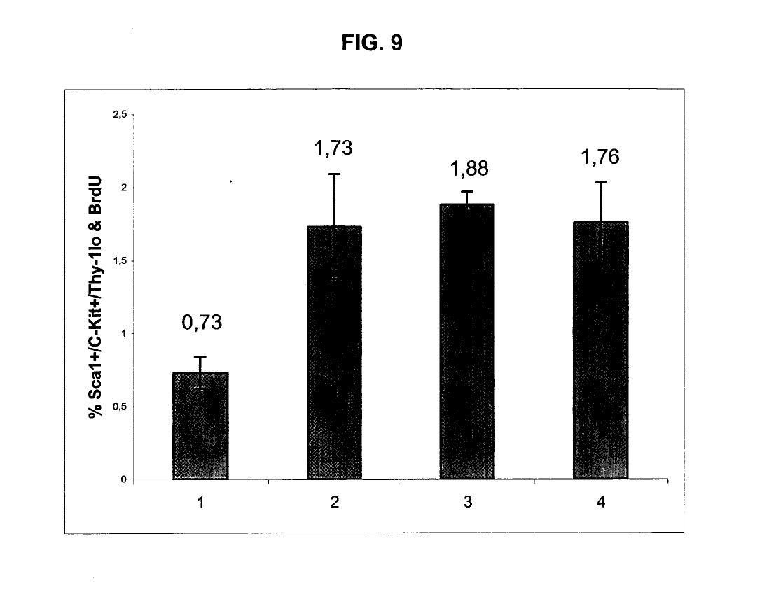

In separate experiments, hematopoietic stem cells (identified as Sca1 positive, c-Kit positive/Thy-1 low) that were also positive for BrdU were quantified by FACS analysis of bone marrow of normal mice exposed to (1) recombinant human IgG-Fc protein; (2) combination of recombinant mouse ephrin-A2-Fc and recombinant mouse ephrin-B2-Fc; (3) recombinant mouse ephrin-A2-Fc; or (4) recombinant mouse ephrin-B2-Fc through i.p. administration for 24 h. Animals were given BrdU and sacrificed 24 hour later as described above. It was found that ephrin-treatment significantly increased the percentage of hematopoietic stem cells that also carried the marker for newborn cells (FIG. 9).

The sum of these experiments indicated that ephrins can influence the activity of ephrin receptors in the signaling pathway, and thereby regulate proliferation, survival, and/or differentiation of stem cells and progenitor cells. This applies to ephrin-A2 and -B2, as well as other ephrins.

### Example 5

Additional tests were performed in vivo. Mice were injected with PBS (phosphate buffered saline) plus Fc (Table 3A, row 1); ephrin-A2-Fc; or the ligand binding domain of Eph A7/GST Fusion. Treatment time was 3 days. Following treatment, the percentage of dividing cells was measured in bone marrow. As shown in Table 3A, both ephrin-A2-Fc and the ligand binding domain showed positive effects; both increased the division of stem cells.

TABLE 3A
Bone Marrow

| BrdU | % | SEM | p-value |
|---|---|---|---|
| PBS + Fc; 90 μg/animal | 56.46 | 1.85 | |
| Ephrin A2-FC; 100 μg/animal | 60.65 | 2.3 | 0.19 |
| Ligand Binding Domain of Eph | 65.8 | 3.58 | 0.03 |
| A7/GST Fusion; 90 μg/animal | | | |

“SEM” indicates standard error of mean.

The same procedure was performed as above. Treatment time was 3 days. Following treatment, the amount of BrdU incorporation in bone marrow stem cells (Sca⁺/c-kit⁺) was measured. As shown in Table 3B, both ephrin-A2-Fc and the ligand binding domain showed positive effects, and increased the division of stem cells.

TABLE 3B
BrdU/Sca⁺/c-kit⁺

| | BrdU | SEM | p-value |
|---|---|---|---|
| PBS + Fc | 447.86 | 49.4 | |
| Ephrin A2-Fc; 100 μg/animal | 674.75 | 83.84 | 0.033 |
| Ligand Binding Domain of | 535.67 | 220.92 | 0.58 |
| Eph A7/GST Fusion; | | | |
| 90 μg/animal | | | |

“SEM” indicates standard error of mean.

The effects of the administration of soluble ephrin and ephrin receptor ligand binding domain on small intestine stem cells were determined. Reagents were administered at levels shown in Table 3C. BrdU incorporation was measured as BrdU levels per square millimeter. As shown in FIG. 3C, PBS and PBS plus Fc protein only had no effect on stem cell proliferation. In contrast, at levels of 1 μg/animal up to 100 g/animal, administration of ephrin-A2-Fc caused a dose-dependent suppression of cellular division. In addition, the administration of the ligand binding domain had similar effects in suppressing cellular division as evidenced by the amount of BrdU incorporation per square millimeter.

TABLE 3C
Small Intestine

| BrdU | BrdU/mm² | SEM |
|---|---|---|
| PBS | 5805 | 421 |
| PBS + Fc; 90 μg/animal | 5958 | 429 |
| Ephrin A2-Fc; 1 μg/animal | 5021 | 203 |
| Ephrin A2-Fc; 10 μg/animal | 2464 | 213 |
| Ephrin A2-Fc; 100 μg/animal | 1301 | 117 |
| Ligand Binding Domain of | 1781 | 215 |
| Eph A7/GST Fusion; | | |
| 90 μg/animal | | |

“SEM” indicates standard error of mean.

Soluble ephrin showed the same effect on skin (Table 3D). BrdU levels were measured in an area the size of a microscopic field (BrdU/MF) after treatment. Table 3D shows that the levels of skin cell proliferation were decreased significantly with the administration of soluble ephrin. The administration of Fc alone, or the administration of PBS alone had no effect.

TABLE 3D
Skin

| BrdU | BrdU/MF | SEM |
|---|---|---|
| PBS | 19.8 | |
| Fc | 19.8 | |
| Ephrin A2-Fc; 1 μg/animal | 15 | 1 |
| Ephrin A2-Fc; 10 μg/animal | 9.9 | |
| Ephrin A2-Fc; 100 μg/animal | 8.6 | |

“SEM” indicates standard error of mean.

Additional experimental data is summarized in Table 4, below. For this data, “+” indicates an increase in stem cell proliferation; “−” indicates a decrease in stem cell proliferation; “n.t.” indicates not tested; and “none” indicates no effect on proliferation.

TABLE 4

| | Brain | Blood | Gut | Skin |
|---|---|---|---|---|
| ephrin-A2-Fc | + | + | − | − |
| ephrin-B2-Fc | + | + | − | − |
| ephrin-A5-Fc | + | n.t. | n.t. | n.t. |
| ephrin-B1-Fc | none | n.t. | n.t. | n.t. |
| EphA7-Fc | + | n.t. | n.t. | n.t. |
| Ligand Binding Domain | n.t | + | − | n.t. |
| IgG-Fc | none | none | none | none |

### REFERENCES

Aasheim H C, Munthe E, Funderud S, Smeland E B, Beiske K, Logtenberg T. (2000). A splice variant of human ephrin-A4 encodes a soluble molecule that is secreted by activated human B lymphocytes. Blood 95, 221-30
Akashi, K., Traver, D., Miyamoto, T., and Weissman, I. L. (2000). A clonogenic common myeloid progenitor that gives rise to all myeloid lineages. Nature 404, 193-197.
Altman, J., and Das, G. D. (1965). Autoradiographic and histological evidence of postnatal neurogenesis in rats. J Comp Neurol 124, 319-335.
Austin, T. W., Solar, G. P., Ziegler, F. C., Liem, L., and Matthews, W. (1997). A role for the Wnt gene family in hematopoiesis: expansion of multilineage progenitor cells. Blood 89, 3624-3635.
Batlle, E., Henderson, J. T., Beghtel, H., van den Born, M. M. W., Sancho, E., Huls, G., Meeldijk, J., Robertson, J., van de Wetering, M., Pawson, T., and Clevers, H. (2002). β-catenin and TCF mediate cell positioning in the intestinal epithelium by controlling the expression of EphB/EphrinB. Cell 111, 251-263.
Bhardwaj, G., Murdoch, B., Wu, D., Baker, D. P., Williams, K. P., Chadwick, K., Ling, L. E., Karanu, F. N., and Bhatia, M. (2001). Sonic hedgehog induces the proliferation of primitive human hematopoietic cells via BMP regulation. Nat Immunol 2, 172-180.
Briscoe, J., and Ericson, J. (2001). Specification of neuronal fates in the ventral neural tube. Curr Opin Neurobiol 11, 43-49.
Brückner, K., Pasquale, E. B., and Klein, R. (1997). Tyrosine phosphorylation of transmembrane ligands for eph receptors. Science 275, 1640-1643.
Cameron, H. A., McEwen, B. S., and Gould, E. (1995). Regulation of adult neurogenesis by excitatory input and NMDA receptor activation in the dentate gyrus. J Neurosci 15, 4687-4692.
Cheng, H.-J., Nakamoto, M., Bergemann, A. D., and Flanagan, J. G. (1995). Complementary gradients in expression and binding of Elf-i and Mek4 in development of the topographic retinotectal projection map. Cell 82, 371-381.
Cheshier, S. H., Morrison, S. J., Liao, X., and Weissman, I. L. (1999). In vivo proliferation and cell cycle kinetics of long-term self-renewing hematopoietic stem cells. Proc Natl Acad Sci USA 96, 3120-3125.
Chin-Sang, I. D., George, S. E., Ding, M., Moseley, S. L., Lynch, A. S., and Chisholm, A. D. (1999). The ephrin VAB-2/EFN-1 functions in neuronal signaling to regulate epidermal morphogenesis in C. elegans. Cell 99, 781-790.
Cooke, J. E., and Moens, C. B. (2002). Boundary formation in the hindbrain: Eph only it were simple. Trends Neurosci 25, 260-267.
Committee, E. N. (1997). Unified nomenclature for Eph family receptors and their ligands, the ephrins. Cell 90, 403-404.
Compton, C. C., Nadire, K. B., Regauer, S., Simon, M., Warland, G., O'Connor, N. E., Gallico, G. G., and Landry, D. B. (1998). Cultured human sole-derived keratinocyte grafts re-express site-specific differentiation after transplantation. Differentiation 64, 45-53.
Conover, J. C., Doetsch, F., Garcia-Verdugo, J. M., Gale, N. W., Yancopoulos, G. D., and Alvarez-Buylla, A. (2000). Disruption of Eph/ephrin signaling affects migration and proliferation in the adult subventricular zone. Nat Neurosci 3, 1091-1097.
Cowan, C. A., and Henkemeyer, M. (2002). Ephrins in reverse, park and drive. Trends Cell Biol 12, 339-346.
Cutforth, T., Moring, L., Mendelsohn, M., Nemes, A., Shah, N. M., Kim, M. M., Frisén, J., and Axel, R. (2003). Axonal ephrin-As and odorant receptors: coordinate determination of the olfactory sensory map. Cell 114, 311-322.
Davis, S., Gale, N. W., Aldrich, T. H., Maisonpierre, P. C., Lhotak, V., Pawson, T., Goldfarb, M., and Yancopoulos, G. D. (1994). Ligands for EPH-related receptor tyrosine kinases that require membrane attachment or clustering for activity. Science 266, 816-819.
Davy, A., Gale, N. W., Murray, E. W., Klinghoffer, R. A., Soriano, P., Feuerstein, C., and Robbins, S. M. (1999). Compartmentalized signaling by GPI-anchored ephrin-A5 requires the Fyn tyrosine kinase to regulate cellular adhesion. Genes Dev 13, 3125-3135.
Davy, A., and Robbins, S. M. (2000). Ephrin-A5 modulates cell adhesion and morphology in an integrin-dependent manner. EMBO J. 19, 5396-5405.
Drescher, U., Kremoser, C., Handwerker, C., Löschinger, J., Noda, M., and Bonhoeffer, F. (1995). In vitro guidance of retinal ganglion cell axons by RAGS, a 25 kDa tectal protein related to ligands for Eph receptor tyrosine kinases. Cell 82, 359-370.
Drescher, U., Bonhoeffer, F., and Muller, B. K. (1997). The Eph family in retinal axon guidance. Curr Opin Neurobiol 7, 75-80.
Durbin, L., Brennan, C., Shiomi, K., Cooke, J., Barrios, A., Shanmugalingam, S., Guthrie, B., Lindberg, R., and Holder, N. (1998). Eph signalling is required for segmentation and differentiation of the somites. Genes and Dev 12, 3096-3109.
Eriksson, P. S., Perfilieva, E., Björk-Eriksson, T., Albom, A. M., Nordborg, C., Peterson, D. A., and Gage, F. H. (1998). Neurogenesis in the adult human hippocampus. Nat Med 4, 1313-1317.
Feldheim, D. A., Kim, Y. I., Bergemann, A. D., Frisén, J., Barbacid, M., and Flanagan, J. G. (2000). Genetic analysis of ephrin-A2 and ephrin-A5 shows their requirement in multiple aspects of retinocollicular mapping. Neuron 25, 563-574.
Feldheim, D. A., Vanderhaeghen, P., Hansen, M. J., Frisén, J., Lu, Q., Barbacid, M., and Flanagan, J. G. (1998). Topographic guidance labels in a sensory projection to the forebrain. Neuron 21, 1303-1313.
Frisén, J., Holmberg, J., and Barbacid, M. (1999). Ephrins and their eph receptors: multitalented directors of embryonic development. EMBO J. 18, 5159-5165.
Frisén, J., Yates, P. A., McLaughlin, T., Friedman, G. C., O'Leary, D. D. M., and Barbacid, M. (1998). Ephrin-A5 (AL-1/RAGS) is essential for proper retinal axon guidance and topographic mapping in the mammalian visual system. Neuron 20, 235-243.
George, S. E., Simokat, K., Hardin, J., and Chisholm, A. D. (1998). The VAB-1 Eph receptor tyrosine kinase functions in neural and epithelial morphogenesis in C. elegans. Cell 92, 633-643.
Goldman, S. A., and Nottebohm, F. (1983). Neuronal production, migration, and differentiation in a vocal control nucleus of the adult female canary brain. Proc Natl Acad Sci USA 80, 2390-2394.
Goodell, M. A., Brose, K., Paradis, G., Conner, A. S., and Mulligan, R. C. (1996). Isolation and functional properties of murine hematopoietic stem cells that are replicating in vivo. J Exp Med 183, 1797-1806.
Gould, E., Reeves, A. J., Graziano, M. S., and Gross, C. G. (1999). Neurogenesis in the neocortex of adult primates. Science 286, 548-552.
Hackney, J. A., Charbord, P., Brunk, B. P., Stoeckert, C. J., Lemischka, I. R., and Moore, K. A. (2002). A molecular profile of a hematopoietic stem cell niche. Proc Natl Acad Sci USA 99, 13061-13066.
Henkemeyer, M., Orioli, D., Henderson, J. T., Saxton, T. M., Roder, J., Pawson, T., and Klein, R. (1996). Nuk controls pathfinding of commissural axons in the mammalian central nervous system. Cell 86, 35-46.
Holland, S. J., Gale, N. W., Mbamalu, G., Yancopoulos, G. D., Henkemeyer, M., and Pawson, T. (1996). Bidirectional signalling through the EPH-family receptor Nuk and its transmembrane ligands. Nature 383, 722-725.
Holmberg, J., and Frisén, J. (2002). Ephrins are not only unattractive. Trends Neurosci 25, 239-243.
Imai, K. S. (2003). Isolation and characterization of beta-catenin downstream genes in early embryos of the ascidian Ciona savignyi. Differentiation 71, 346-360.
Ivanova, N. B., Dimos, J. T., Schaniel, C., Hackney, J. A., Moore, K. A., and Lemischka, I. R. (2002). A stem cell molecular signature. Science 298, 601-604.
Janes, S. M., Lowell, S., and Hutter, C. (2002). Epidermal stem cells. J Pathol 197, 479-491.
Kaplan, M. S. (1981). Neurogenesis in the 3-month-old rat visual cortex. J Comp Neurol 195, 323-338.
Karanu, F. N., Murdoch, B., Gallacher, L., Wu, D. M., Koremoto, M., Sakano, S., and Bhatia, M. (2000). The notch ligand jagged-1 represents a novel growth factor of human hematopoietic stem cells. J Exp Med 192, 1365-1372.
Kondo, M., Scherer, D. C., King, A. G., Manz, M. G., and Weissman, I. L. (2001). Lymphocyte development from hematopoietic stem cells. Curr Opin Genet Dev 11, 520-526.
Kondo, M., Wagers, A. J., Manz, M. G., Prohaska, S. S., Scherer, D. C., Beilhack, G. F., Shizuru, J. A., and Weissman, I. L. (2003). Biology of hematopoietic stem cells and progenitors: implications for clinical application. Annu Rev Immunol 21, 759-806.
Kondo, M., Weissman, I. L., and Akashi, K. (1997). Identification of clonogenic common lymphoid progenitors in mouse bone marrow. Cell 91, 661-672.
Korinek, V., Barker, N., Moerer, P., van Donselaar, E., Huls, G., Peters, P. J., and Clevers, H. (1998). Depletion of epithelial stem-cell compartments in the small intestine of mice lacking Tcf-4. Nat Genet 19, 379-383.
Krumlauf, R. (1994). Hox genes in vertbrate development. Cell 78, 191-201.
Kullander, K., and Klein, R. (2002). Mechanisms and functions of Eph and ephrin signalling. Nat Rev Mol Cell Biol 3, 475-486.
Lhotak, V., and Pawson, T. (1993). Biological and biochemical activities of a chimeric epidermal growth factor-elk receptor tyrosine kinase. Mol Cell Biol 13, 7071-7079.
Lumsden, A., and Krumlauf, R. (1996). Patterning the vertebrate neuraxis. Science 274, 1109-1115.
Luskin, M. B. (1993). Restricted proliferation and migration of postnataly generated neurons derived from the forebrain subventricular zone. Neuron 11, 173-189.
Lois, C., and Alvarez-Buylla, A. (1993). Proliferating subventricular zone cells in the adult mammalian forebrain can differentiate into neurons and glia. Proc Natl Acad Sci USA 90, 2074-2077.
Marshman, E., Booth, C., and Potten, C. S. (2002). The intestinal epithelial stem cell. Bioessays 24, 91-98.
Meima, L., Kljavin, I. J., Moran, P., Shih, A., Winslow, J. W., and Caras, I. W. (1997). AL-1-induced growth cone collapse of rat cortical neurons is correlated with REK7 expression and rearrangement of the actin cytoskeleton. Eur J Neurosci 9, 177-188.
Mellitzer, G., Xu, Q., and Wilkinson, D. G. (1999). Eph receptors and ephrins restrict cell intermingling and communication. Nature 400, 77-81.
Miller, M. A., Ruest, P. J., Kosinski, M., Hanks, S. K., and Greenstein, D. (2003). An Eph receptor sperm-sensing control mechanism for oocyte meiotic maturation in Caenorhabditis elegans. Genes and Dev 17, 187-200.
Momma, S., Johansson, C. B., and Frisén, J. (2000). Get to know your stem cells. Curr Opin Neurobiol 10, 45-49.
Nakorn, T. N., Miyamoto, T., and Weissman, I. L. (2003). Characterization of mouse clonogenic megakaryocyte progenitors. Proc Natl Acad Sci USA 100, 205-210.
Nicolas, M., Wolfer, A., Raj, K., Kummer, J. A., Mill, P., van Noort, M., Hui, C. C., Clevers, H., Dotto, G. P., and Radtke, F. (2003). Notchl functions as a tumor suppressor in mouse skin. Nat Genet 33, 416-421.
Palmer, A., and Klein, R. (2003). Multiple roles of ephrins in morphogenesis, neuronal networking, and brain function. Genes Dev 17, 1429-1450.
Pinto, D., Gregorieff, A., Begthel, H., and Clevers, H. (2003). Canonical Wnt signals are essential for homeostasis of the intestinal epithelium. Genes Dev 17, 1709-1713.
Rakic, P. (2002). Neurogenesis in adult primate neocortex: an evaluation of the evidence. Nat Rev Neurosci 3, 65-71.
Ramalho-Santos, M., Yoon, S., Matsuzaki, Y., Mulligan, R. C., and Melton, D. A. (2002). “Stemness”: transcriptional profiling of embryonic and adult stem cells. Science 298, 597-600.
Sauvageau, G., Thorsteinsdottir, U., Eaves, C. J., Lawrence, H. J., Largman, C., Lansdorp, P.

M., and Humphries, R. K. (1995). Overexpression of HOXB4 in hematopoietic cells causes the selective expansion of more primitive populations in vitro and in vivo. Genes Dev 9, 1753-1765.
Screpanti, I., Bellavia, D., Campese, A. F., Frati, L., and Gulino, A. (2003). Notch, a unifying target in T-cell acute lymphoblastic leukemia? Trends Mol Med 9, 30-35.
Smith, A., Robinson, V., Patel, K., and Wilkinson, D. G. (1997). The EphA4 and EphB1 receptor tyrosine kinases and ephrin-B2 ligand regulate targeted migration of branchial neural crest cells. Curr Biol 7, 561-570.
Sperry, R. W. (1963). Chemoaffinity in the orderly growth of nerve fiber patterns and connections. Proc Natl Acad Sci USA 50, 703-710.
Stephenson, S. A., Slomka, S., Douglas, E. L., Hewett, P. J., and Hardingham, J. E. (2001). Receptor protein tyrosine kinase EphB4 is up-regulated in colon cancer. BMC Mol Biol 2, 15.
Temple, S., and Alvarez-Buylla, A. (1999). Stem cells in the adult mammalian central nervous system. Curr Opin Neurobiol 9, 135-141.
Thorsteinsdottir, U., Mamo, A., Kroon, E., Jerome, L., Bijl, J., Lawrence, H. J., Humphries, K., and Sauvageau, G. (2002). Overexpression of the myeloid leukemia-associated Hoxa9 gene in bone marrow cells induces stem cell expansion. Blood 99, 121-129.
Thorsteinsdottir, U., Sauvageau, G., and Humphries, R. K. (1999). Enhanced in vivo regenerative potential of HOXB4-transduced hematopoietic stem cells with regulation of their pool size. Blood 94, 2605-2612.
van der Geer, P., Hunter, T., and Lindberg, R. A. (1994). Receptor protein-tyrosine kinases and their signal transduction pathways. Annu Rev Cell Biol 10, 251-337.
van de Wetering, M., Sancho, E., Verweij, C., de Lau, W., Oving, I., Hurlstone, A., van der Horn, K., Batlle, E., Coudreuse, D., Haramis, A. P., et al. (2002). The beta-catenin/TCF-4 complex imposes a crypt progenitor phenotype on colorectal cancer cells. Cell 111, 241-250.
Vanderhaeghen, P., Lu, Q., Prakash, N., Frisén, J., Walsh, C. A., Frostig, R. D., and Flanagan, J. G. (2000). A mapping label required for normal scale of body representation in the cortex. Nat Neurosci 3, 358-365.
Varnum-Finney, B., Xu, L., Brashem-Stein, C., Nourigat, C., Flowers, D., Bakkour, S., Pear, W. S., and Bernstein, I. D. (2000). Pluripotent, cytokine-dependent, hematopoietic stem cells are immortalized by constitutive Notchl signaling. Nat Med 6, 1278-1281.
Wang, X., Roy, P. J., Holland, S. J., Zhang, L. W., Culotti, J. G., and Pawson, T. (1999). Multiple ephrins control cell organization in C. elegans using kinase-dependent and—independent functions of the VAB-1 Eph receptor. Mol Cell 4, 903-913.
Watt, F. M. (2001). Stem cell fate and patterning in mammalian epidermis. Curr Opin Genet Dev 11, 410-417.
Weiss, S., Dunne, C., Hewson, J., Wohl, C., Wheatley, M., Peterson, A. C., and Reynolds, B. R. (1996). Multipotent CNS stem cells are present in the adult mammalian spinal cord and ventricular neuroaxis. J Neurosci 16, 7599-7609.
Weissman, I. L. (2000). Stem cells: units of development, units of regeneration, and units in evolution. Cell 100, 157-168.
Wilkinson, D. G. (2001). Multiple roles of EPH receptors and ephrins in neural development. Nat Rev Neurosci 2, 155-164.
Xu, Q., Mellitzer, G., Robinson, V., and Wilkinson, D. G. (1999). In vivo cell sorting in complementary segmental domains mediated by Eph receptors and ephrins. Nature 399, 267-271.
Xu, Q. L., Alldus, G., Holder, N., and Wilkinson, D. G. (1995). Expression of truncated Sek-1 receptor tyrosine kinase disrupts the segmental restriction of gene expression in the Xenopus and zebrafish forebrain. Development 121, 4005-4016.

1. A method of alleviating a symptom of a disorder characterized by reduced levels of hematopoiesis comprising: administering an ephrin inhibitor selected from the group consisting of a soluble ephrin and a small molecule to a patient suffering from reduced levels of hematopoiesis, wherein the administered soluble ephrin or small molecule increases proliferation of hematopoietic cells, thereby alleviating the symptom of the disorder.

2. The method of claim 1, wherein the soluble ephrin is selected from the group consisting of soluble ephrin-A2 and soluble ephrin-B2.

3. The method of claim 1, wherein the soluble ephrin or a fragment thereof is joined to a heterologous amino acid sequence.

4. The method of claim 3, wherein the heterologous amino acid sequence comprises a constant domain of an immunoglobulin.

5. The method of claim 1, wherein the disorder is selected from the group consisting of leukopenia, lymphocytopenia, neutropenia, granulocytopenia, agranulocytosis, thrombocytopenia, coagulation factor deficiencies, hypoproliferative anemias, hypoplastic anemias, cancer-induced anemias, chemotherapy-induced anemias, radiation-induced anemias, sepsis-induced anemias, Fanconi's anemia, and anemia associated with Blackfan-Diamond syndrome.

6. The method of claim 1, wherein the soluble ephrin is administered in an amount selected from the group consisting of at least 0.1 ng/kg/day, at least 1 ng/kg/day, at least 5 mg/kg/day, at least 10 mg/kg/day, and at least 50 mg/kg/day.

7. The method of claim 1, wherein the soluble ephrin is locally administered to bone marrow.

8. The method of claim 1, wherein the soluble ephrin is administered to achieve a tissue concentration of 0.11 nM to 100 nM.

9. The method of claim 1, wherein the soluble ephrin is administered by injection.

10. The method of claim 1, wherein the soluble ephrin is administered by a route selected from the group consisting of oral, subcutaneous, intraperitoneal, intramuscular, intracerebroventricular, intraparenchymal, intrathecal, intracranial, buccal, mucosal, nasal, and rectal administration.

11. The method of claim 1, wherein the soluble ephrin is formulated into a pharmaceutical composition comprising a physiologically acceptable carrier, excipient, or diluent.

12. A method of alleviating a symptom of a disorder characterized by reduced levels of hematopoiesis comprising: administering an antibody or affibody that specifically binds to an ephrin or ephrin receptor to a patient suffering from reduced levels of hematopoiesis, wherein the administered antibody or affibody increases proliferation of hematopoietic cells, thereby alleviating a symptom of the disorder.

13. The method of claim 12, wherein the ephrin is selected from the group consisting of ephrin-A2 and ephrin-B2.

14. The method of claim 12, wherein the disorder is selected from the group consisting of leukopenia, lymphocytopenia, neutropenia, granulocytopenia, agranulocytosis, thrombocytopenia, coagulation factor deficiencies, hypoproliferative anemias, hypoplastic anemias, cancer-induced anemias, chemotherapy-induced anemias, radiation-induced anemias, sepsis-induced anemias, Fanconi's anemia, and anemia associated with Blackfan-Diamond syndrome.

15. The method of claim 12, wherein the antibody is selected from the group consisting of polyclonal, monoclonal, chimeric, oligomeric, single chain, F_{ab}, F_{ab′}, and F_{(ab′)2} antibodies.

16. The method of claim 12, wherein the antibody or affibody is administered in an amount selected from the group consisting of at least 0.1 ng/kg/day, at least 1 ng/kg/day, at least 5 mg/kg/day, at least 10 mg/kg/day, and at least 50 mg/kg/day.

17. The method of claim 12, wherein the antibody or affibody is locally administered to bone marrow.

18. The method of claim 12, wherein the antibody or affibody is administered to achieve a tissue concentration of 0.11 nM to 100 nM.

19. The method of claim 12, wherein the antibody or affibody is administered by injection.

20. The method of claim 12, wherein the antibody or affibody is administered by a route selected from the group consisting of oral, subcutaneous, intraperitoneal, intramuscular, intracerebroventricular, intraparenchymal, intrathecal, intracranial, buccal, mucosal, nasal, and rectal administration.

21. The method of claim 12, wherein the antibody or affibody is formulated into a pharmaceutical composition comprising a physiologically acceptable carrier, excipient, or diluent.

22. A method of alleviating a symptom of a disorder characterized by increased levels of cellular proliferation in an intestinal tract comprising: administering an ephrin inhibitor selected from the group consisting of a soluble ephrin and a small molecule to a patient suffering from increased levels of cellular proliferation in a intestinal tract, wherein the administered soluble ephrin or small molecule reduces proliferation of intestinal cells, thereby alleviating the symptom of the disorder.

23. The method of claim 22, wherein the soluble ephrin is selected from the group consisting of a soluble ephrin-A2 and soluble ephrin-B2.

24. The method of claim 22, wherein the soluble ephrin or a fragment thereof is joined to a heterologous amino acid sequence.

25. The method of claim 24, wherein the heterologous amino acid sequence comprises a constant domain of an immunoglobulin.

26. The method of claim 22, wherein the disorder is selected from the group consisting of growths and polyps of the large intestine, colorectal cancers, anorectal cancers, small intestine tumors, Gardner's syndrome, Peutz-Jeghers syndrome, Rendu-Osler-Weber syndrome, Bowen's disease, Crohm disease, ulcerative colitis, irritable bowel syndrome and extramammary Paget's disease.

27. The method of claim 22, wherein the soluble ephrin is administered in an amount selected from the group consisting of at least 0.1 ng/kg/day, at least 1 ng/kg/day, at least 5 mg/kg/day, at least 10 mg/kg/day, and at least 50 mg/kg/day.

28. The method of claim 22, wherein the soluble ephrin is locally administered to an intestinal tract tissue.

29. The method of claim 22, wherein the soluble ephrin is administered to achieve a tissue concentration of 0.1 nM to 100 nM.

30. The method of claim 22, wherein the soluble ephrin is administered by injection.

31. The method of claim 22, wherein the soluble ephrin is administered by a route selected from the group consisting of oral, subcutaneous, intraperitoneal, intramuscular, intracerebroventricular, intraparenchymal, intrathecal, intracranial, buccal, mucosal, nasal, and rectal administration.

32. The method of claim 22, wherein the soluble ephrin is formulated into a pharmaceutical composition comprising a physiologically acceptable carrier, excipient, or diluent.

33. A method of alleviating a symptom of a disorder characterized by increased levels of cellular proliferation in an intestinal tract comprising: administering an antibody or affibody that specifically binds to an ephrin or ephrin receptor to a patient suffering from increased levels of cellular proliferation in an intestinal tract, wherein the administered antibody or affibody reduces proliferation of intestinal cells, thereby treating the disease or disorder.

34. The method of claim 33, wherein the ephrin is selected from the group consisting of ephrin-A2 and ephrin-B2.

35. The method of claim 33, wherein the disorder is selected from the group consisting of growths and polyps of the large intestine, colorectal cancers, anorectal cancers, small intestine tumors, Gardner's syndrome, Peutz-Jeghers syndrome, Rendu-Osler-Weber syndrome, Bowen's disease, and extramammary Paget's disease.

36. The method of claim 33, wherein the antibody is selected from the group consisting of polyclonal, monoclonal, chimeric, oligomeric, single chain, F_{ab}, F_{ab′}, and F_{(ab′)2} antibodies.

37. The method of claim 33, wherein the antibody or affibody is administered in an amount selected from the group consisting of at least 0.1 ng/kg/day, at least 1 ng/kg/day, at least 5 mg/kg/day, at least 10 mg/kg/day, and at least 50 mg/kg/day.

38. The method of claim 33, wherein the antibody or affibody is locally administered to intestinal tract tissue.

39. The method of claim 33, wherein the antibody or affibody is administered to achieve a tissue concentration of 0.1 nM to 100 nM.

40. The method of claim 33, wherein the antibody or affibody is administered by injection.

41. The method of claim 33, wherein the antibody or affibody is administered by a route selected from the group consisting of oral, subcutaneous, intraperitoneal, intramuscular, intracerebroventricular, intraparenchymal, intrathecal, intracranial, buccal, mucosal, nasal, and rectal administration.

42. The method of claim 33, wherein the antibody or affibody is formulated into a pharmaceutical composition comprising a physiologically acceptable carrier, excipient, or diluent.

43. A method of alleviating a symptom of a disorder characterized by an abnormal level of cellular proliferation in a tissue: administering an ephrin inhibitor selected from the group consisting of a soluble ephrin and a small molecule to a patient suffering from abnormal levels of cellular proliferation in the tissue, wherein the administered soluble ephrin or small molecule modulates proliferation of cells in the tissue, thereby alleviating the symptom of the disorder.

44. The method of claim 43 wherein the tissue is selected from the group consisting of skin, retina, prostate, and ovarian tissue.

45. The method of claim 43, wherein the soluble ephrin is selected from the group consisting of a soluble ephrin-A2 and soluble ephrin-B2.

46. The method of claim 43, wherein the soluble ephrin or a fragment thereof is joined to a heterologous amino acid sequence.

47. The method of claim 46, wherein the heterologous amino acid sequence comprises a constant domain of an immunoglobulin.

48. The method of claim 43, wherein the disorder is selected from the group consisting of psoriasis, inflammatory skin disease, skin cancer, skin atrophy, benign prostate hypoplasia, prostate cancer, polycystic ovary syndrome, ovarian cancer.

49. The method of claim 43, wherein the soluble ephrin is administered in an amount selected from the group consisting of at least 0.1 ng/kg/day, at least 1 ng/kg/day, at least 5 mg/kg/day, at least 10 mg/kg/day, and at least 50 mg/kg/day.

50. The method of claim 43, wherein the soluble ephrin is locally administered to a tissue selected from the group consisting of skin, prostate, and ovarian tissue.

51. The method of claim 43, wherein the soluble ephrin is administered to achieve a tissue concentration of 0.11 nM to 100 nM.

52. The method of claim 42, wherein the soluble ephrin is administered by injection.

53. The method of claim 43, wherein the soluble ephrin is administered by a route selected from the group consisting of oral, subcutaneous, intraperitoneal, intramuscular, intracerebroventricular, intraparenchymal, intrathecal, intracranial, buccal, mucosal, nasal, and rectal administration.

54. The method of claim 43, wherein the soluble ephrin is formulated into a pharmaceutical composition comprising a physiologically acceptable carrier, excipient, or diluent.

55. A method for alleviating a symptom of a disorder characterized by abnormal levels of cellular proliferation in a tissue comprising: administering a soluble ephrin receptor to a patient suffering said disorder, wherein the administered soluble ephrin receptor modulates proliferation of cells in the tissue, thereby alleviating the symptom of the disorder.

56. The method of claim 55 wherein the soluble ephrin receptor comprises a ligand binding domain of an ephrin receptor.

57. The method of claim 55, wherein the soluble ephrin receptor or a fragment thereof is joined to a heterologous amino acid sequence.

58. The method of claim 57, wherein the heterologous amino acid sequence comprises a constant domain of an immunoglobulin.


SEQUENCE LISTING (25 sequences)

SEQ ID NO: 1 - protein, 205 aa, Mus musculus
MEFLWAPLLGLCCSLAAADRHIVFWNSSNPKFREEDYTVHVQLNDYLDIICPHYEDDSVADAAMERYTLYMVEHQEYVACQPQSKDQVRWNCNRPSAKHGPEKLSVKFQRFTPFILGKEFKEGHSYYYISKPIYHQESQCLKLKVTVNGKITHNPQAHVNPQEKRLQADDPEVQVLHSIGYSAAPRLFPLVWAVLLLPLLLLQSQ

SEQ ID NO: 2 - protein, 209 aa, Mus musculus
MAPAQRPLLPLLLLLLPLRARNEDPARANADRYAVYWNRSNPRFQVSAVGDGGGYTVEVSINDYLDIYCPHYGAPLPPAERMERYILYMVNGEGHASCDHRQRGFKRWECNRPAAPGGPLKFSEKFQLFTPFSLGFEFRPGHEYYYISATPPNLVDRPCLRLKVYVRPTNETLYEAPEPIFTSNSSCSGLGGCHLFLTTVPVLWSLLGS

SEQ ID NO: 3 - protein, 230 aa, Mus musculus
MAAAPLLLLLLLVPVPLLPLLAQGPGGALGNRHAVYWNSSNQHLRREGYTVQVNVNDYLDIYCPHYNSSGPGGGAEQYVLYMVNLSGYRTCNASQGSKRWECNRQHASHSPIKFSEKFQRYSAFSLGYEFHAGQEYYYISTPTHNLHWKCLRMKVFVCCASTSHSGEKPVPTLPQFTMGPNVKINVLEDFEGENPQVPKLEKSISGTSPKREHLPLAVGIAFFLMTLLAS

SEQ ID NO: 4 - protein, 206 aa, Mus musculus
MRLLPLLRTVLWAALLGSRLPGCSSLRHPIYWNSSNPRLLRGDAVVELGFNDYLDIFCPHYESPGPPEGPETFALYMVDWSGYEACTAEGANAFQRWNCSMPFAPFSPVRFSEKIQRYTPFPLGFEFLPGETYYYISVPTPESPGRCLRLQVSVCCKESGSSHESAHPVGSPGESGTSGWRGGHAPSPLCLLLLLLLPILRLLRVL

SEQ ID NO: 5 - protein, 228 aa, Mus musculus
MLHVEMLTLLFLVLWMCVFSQDPGSKVVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTVHESAEPSRGENAAQTPRIPSRLLAILLFLLAMLLTL

SEQ ID NO: 6 - protein, 345 aa, Mus musculus
MARPGQRWLSKWLVAMVVLTLCRLATPLAKNLEPVSWSSLNPKFLSGKGLVIYPKIGDKLDIICPRAEAGRPYEYYKLYLVRPEQAAACSTVLDPNVLVTCNKPHQEIRFTIKFQEFSPNYMGLEFKKYHDYYITSTSNGSLEGLENREGGVCRTRTMKIVMKVGQDPNAVTPEQLTTSRPSKESDNTVKTATQAPGRGSQGDSDGKHETVNQEEKSGPGAGGGGSGDSDSFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 7 - protein, 336 aa, Mus musculus
MAMARSRRDSVWKYCWGLLMVLCRTAISRSIVLEPIYWNSSNSKFLPGQGLVLYPQIGDKLDIICPKVDSKTVGQYEYYKVYMVDKDQADRCTIKKENTPLLNCARPDQDVKFTIKFQEFSPNLWGLEFQKNKDYYIISTSNGSLEGLDNQEGGVCQTRAMKILMKVGQDASSAGSARNHGPTRRPELEAGTNGRSSTTSPFVKPNPGSSTDGNSAGHSGNNLLGSEVALFAGIASGCIIFIVIIITLVVLLLKYRRRHRKHSPQHTTTLSLSTLATPKRGGNNNGSEPSDVIIPLRTADSVFCPHYEKVSGDYGHPVYIVQEMPPQSPANIYYKV

SEQ ID NO: 8 - protein, 340 aa, Mus musculus
MGAPHFGPGGVQVGALLLLGFAGLVSGLSLEPVYWNSANKRFQAEGGYVLYPQIGDRLDLLCPRARPPGPHSSPSYEFYKLYLVEGAQGRRCEAPPAPNLLLTCDRPDLDLRFTIKFQEYSPNLWGHEFRSHHDYYIIATSDGTREGLESLQGGVCLTRGMKVLLRVGQSPRGGAVPRKPVSEMPMERDRGAAHSAEPGRDTIPGDPSSNATSRGAEGPLPPPSMPAVAGAAGGMALLLLGVAGAGGAMCWRRRRAKPSESRHPGPGSFGRGGSLGLGGGGGMGPREAEPGELGIALRGGGTADPPFCPHYEKVSGDYGHPVYIVQDGPPQSPPNIYYKV

SEQ ID NO: 9 - protein, 205 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKVTVSGKITHSPQAHVNPQEKRLAADDPEVRVLHSIGHSAAPRLFPLAWTVLLLPLLLLQTP

SEQ ID NO: 10 - protein, 213 aa, Homo sapiens
MAPAQRPLLPLLLLLLPLPPPPFARAEDAARANSDRYAVYWNRSNPRFHAGAGDDGGGYTVEVSINDYLDIYCPHYGAPLPPAERMEHYVLYMVNGEGHASCDHRQRGFKRWECNRPAAPGGPLKFSEKFQLFTPFSLGFEFRPGHEYYYISATPPNAVDRPCLRLKVYVRPTNETLYEAPEPIFTSNNSCSSPGGCRLFLSTIPVLWTLLGS

SEQ ID NO: 11 - protein, 238 aa, Homo sapiens
MAAAPLLLLLLLVPVPLLPLLAQGPGGALGNRHAVYWNSSNQHLRREGYTVQVNVNDYLDIYCPHYNSSGVGPGAGPGPGGGAEQYVLYMVSRNGYRTCNASQGFKRWECNRPHAPHSPIKFSEKFQRYSAFSLGYEFHAGHEYYYISTPTHNLHWKCLRMKVFVCCASTSHSGEKPVPTLPQFTMGPNVKINVLEDFEGENPQVPKLEKSISGTSPKREHLPLAVGIAFFLMTFLAS

SEQ ID NO: 12 - protein, 201 aa, Homo sapiens
MRLLPLLRTVLWAAFLGSPLRGGSSLRHVVYWNSSNPRLLRGDAVVELGLNDYLDIVCPHYEGPGPPEGPETFALYMVDWPGYESCQAEGPRAYKRWVCSLPFGHVQFSEKIQRFTPFSLGFEFLPGETYYYISVPTPESSGQCLRLQVSVCCKERKSESAHPVGSPGESGTSGWRGGDTPSPLCLLLLLLLLILRLLRIL

SEQ ID NO: 13 - protein, 228 aa, Homo sapiens
MLHVEMLTLVFLVLWMCVFSQDPGSKAVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTVHESAEPSRGENAAQTPRIPSRLLAILLFLLAMLLTL

SEQ ID NO: 14 - protein, 346 aa, Homo sapiens
MARPGQRWLGKWLVAMVVWALCRLATPLAKNLEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTSNGSLEGLENREGGVCRTRTMKIIMKVGQDPNAVTPEQLTTSRPSKEADNTVKMATQAPGSRGSLGDSDGKHETVNQEEKSGPGASGGSSGDPDGFFNSKVALFAAVGAGCVIFLLIIIFLTVLLLKLRKRHRKHTQQRAAALSLSTLASPKGGSGTAGTEPSDIIIPLRTTENNYCPHYEKVSGDYGHPVYIVQEMPPQSPANIYYKV

SEQ ID NO: 15 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 16 - protein, 340 aa, Homo sapiens
MGPPHSGPGGVRVGALLLLGVLGLVSGLSLEPVYWNSANKRFQAEGGYVLYPQIGDRLDLLCPRARPPGPHSSPNYEFYKLYLVGGAQGRRCEAPPAPNLLLTCDRPDLDLRFTIKFQEYSPNLWGHEFRSHHDYYIIATSDGTREGLESLQGGVCLTRGMKVLLRVGQSPRGGAVPRKPVSEMPMERDRGAAHSLEPGKENLPGDPTSNATSRGAEGPLPPPSMPAVAGAAGGLALLLLGVAGAGGAMCWRRRRAKPSESRHPGPGSFGRGGSLGLGGGGGMGPREAEPGELGIALRGGGAADPPFCPHYEKVSGDYGHPVYIVQDGPPQSPPNIYYKV

SEQ ID NO: 17 - protein, 417 aa, Artificial
  GST-EphA7-LBD fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFPGEVLLLDSKAQQTELEWISSPPNGWEEISGLDENYTPIRTYQVCQVMEPNQNNWLRTNWISKGNAQRIFVELKFTLRDCNSLPGVLGTCKETFNLYYYETDYDTGRNIRENLYVKIDTIAADESFTQGDLGERKMKLNTEVREIGPLSKKGFYLAFQDVGACIALVSVKVYYKKCWSIIELERPHRD

SEQ ID NO: 18 - protein, 184 aa, Mouse
MAPAQRPLLPLLLLLLPLRARNEDPARANADRYAVYWNRSNPRFQVSAVGDGGGYTVEVSINDYLDIYCPHYGAPLPPAERMERYILYMVNGEGHASCDHRQRGFKRWECNRPAAPGGPLKFSEKFQLFTPFSLGFEFRPGHEYYYISATPPNLVDRPCLRLKVYVRPTNETLYEAPEPIFTSN

SEQ ID NO: 19 - protein, 188 aa, Human
MAPAQAPLLPLLLLLLPLPPPPFAPPEDRRRANSDRYAVYWNRSNPRFHAGAGDDGGGYTVEVSINDYLDIYCPHYGAPLPPAERMEHYVLYMVNGEGHASCDHRQRGFKRWECNRPAAPGGPLKFSEKFQLFTPFSLGFEFRPGHEYYYISATPPNAVDRPCLRLKVYVRPTNETLYEAPEPIFTSN

SEQ ID NO: 20 - protein, 229 aa, Mouse
MAMARSRRDSVWKYCWGLLMVLCRTAISRSIVLEPIYWNSSNSKFLPGQGLVLYPQIGDKLDIICPKVDSKTVGQYEYYKVYMVDKDQADRCTIKKENTPLLNCARPDQDVKFTIKFQEFSPNLWGLEFQKNKDYYIISTSNGSLEGLDNQEGGVCQTRAMKILMKVGQDASSAGSARNHGPTRRPELEAGTNGRSSTTSPFVKPNPGSSTDGNSAGHSGNNLLGSEVA

SEQ ID NO: 21 - protein, 226 aa, Human
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVA

SEQ ID NO: 22 - protein, 330 aa, Homo sapiens [sequence omitted]

SEQ ID NO: 23 - protein, 4 aa, Mus musculus
MRLL

SEQ ID NO: 24 - protein, 22 aa, Mus musculus
MLLRLGLIYPPTRPPAPPGPLV

SEQ ID NO: 25 - protein, 4 aa, Homo sapiens
VGPG